(19) United States
(12) Patent Application Publication
(10) Pub. No.: US 2023/0404980 A1
(43) Pub. Date: Dec. 21, 2023

(54) METHODS OF TREATING SPONDYLOARTHRITIS OR SYMPTOMS THEREOF

(71) Applicant: UNIVERSITY HEALTH NETWORK, Toronto (CA)

(72) Inventors: Nigil Haroon, Toronto (CA); Akihiro Nakamura, Toronto (CA)

(21) Appl. No.: 18/033,994

(22) PCT Filed: Oct. 19, 2021
(86) PCT No.: PCT/CA2021/051470

§ 371 Date: Apr. 26, 2023

Related U.S. Application Data
(60) Provisional application No. 63/106,859, filed on Oct. 28, 2020.

(51) Int. Cl. A61K 31/423; C07K 16/24; C07K 16/28; A61P 19/02

(52) U.S. Cl. CPC A61K 31/423; C07K 16/24; C07K 16/2833; A61P 19/02

(57) ABSTRACT

Provided are methods of treating SpA, uses for treating SpA and compositions for treating SpA. The methods involve administering a MIF inhibitor to a subject in need thereof. The MIF inhibitor can be a compound or an anti-MIF antibody.

### CROSS-REFERENCE TO RELATED APPLICATIONS

This PCT application claims priority to U.S. Provisional Patent Application No. 63/106,859 filed Oct. 28, 2020, the contents of which are incorporated herein by reference.

### FIELD

The present disclosure relates to MIF inhibitors and specifically to their use in treating spondyloarthritis or symptoms thereof.

### BACKGROUND

Spondyloarthritis (SpA) is a chronic rheumatic disease characterized by severe inflammation in distinct anatomical sites and abnormal new bone formation (NBF) in the entheses of the spine and peripheral joints (1). Uncontrolled inflammation with mechanical stimulation facilitates NBF via endochondral ossification (ECO), and eventual ankylosis (2), contributing to severe pain and restriction in physical activities. Although inhibitors of tumor necrosis factor (TNF) and interleukin (IL)-17 are approved for treatment, up to 40% of SpA patients do not adequately respond to any therapeutic modality (3, 4). Furthermore, there is a challenge remaining in adequately controlling various SpA-associated extra-articular symptoms including psoriasis, colitis, and uveitis with currently available therapy.

MIF is an upstream immuno-regulatory cytokine that promotes inflammation and influences the differentiation of the adaptive immune response (5). Serum levels of MIF are increased in a number of inflammatory conditions including ankylosing spondylitis (AS), a subset of SpA (6). Autoantibodies directed against the MIF cognate receptor CD74 are also present in the serum of SpA patients((7-9). Moreover, CD4+ T cells from SpA patients produce more inflammatory cytokines in response to recombinant CD74 stimulation than those from rheumatoid arthritis (RA) or healthy donors (10).

AS patients have significantly higher levels of MIF in serum, synovial fluid, or gut tissues compared to healthy individuals or osteoarthritis disease controls (6). Baseline MIF levels in the serum independently predict radiographic progression of AS patients (6). Interestingly, overexpression of MIF did not induce clear SpA pathologies in wild type C57BL/6 or BALB/c mice.

The major contributors towards inflammation and NBF in SpA are type 3 immune cells including T helper 17 (Th17) lineage cells and group 3 innate lymphoid cells (ILC3s) that produce IL-17A A and IL-22 in both axial and peripheral joint tissues (11, 12). It is well-acknowledged that T cells undergo polarized differentiation from naïve CD4+ T cells into subpopulations such as Th1, Th2 and Th17, an outcome highly dependent on the cytokine microenvironment present during T cell activation (13, 14). It is also well-established that naïve CD4+ T cells can differentiate into CD25+Foxp3+ regulatory T cells (Tregs). In RA, Th17 cells with arthritogenic and autoreactive properties can arise from Tregs (15).

The SKG strain of mice, with a Zap70 point mutation on the BALB/c genetic background, develops chronic arthritis, enthesitis, sacroiliitis, spinal inflammation and extra-articular manifestations through T cell activation (16, 17); thus, the SKG mouse is a well-established mouse model of SpA (18, 19).

Inhibitors of tumor necrosis factor (TNF) and interleukin (IL)-17 are approved for treatment. However, up to 40% of SpA patients do not adequately respond to any therapeutic modality (3, 4) and available treatments do not uniformly control SpA-associated extra-articular symptoms including psoriasis, colitis, and uveitis. Treatments such as methotrexate, Leflunomide, sulfasalazine, inhibitors of IL-6 signaling (tocilizumab) and B cells (rituximab) as well as blockers of T cell co-stimulation (abatacept) effective in RA treatment, are not useful in SpA.

Accordingly, there is a need for additional treatments for SpA as well as its associated extra-articular symptoms.

### SUMMARY

As demonstrated herein, MIF inhibitors have been shown to inhibit Spondyloarthritis (SpA) and associated extra-articular manifestations. the. The inventors found that the expression of MIF and its receptor CD74 were significantly increased in blood and target tissues of curdlan-treated SKG mice, as compared to control SKG mice. Overexpression of MIF in vivo was sufficient to recapitulate major SpA clinical manifestations, whereas genetic deletion of MIF using Mif knockout (KO) SKG mice or antagonist blockade suppressed SpA-related pathology. Using these mouse models, the inventors found that MIF plays a critical role in expansion of Th17 cells, ILC3s and inflammatory macrophages. Neutrophils were found to be substantially expanded and to express MIF in curdlan-treated SKG mice, and to be sufficient to induce SpA pathology in Mif KO SKG mice. Strikingly, MIF boosted acquisition of a Th17 cell-like phenotype from Tregs in both mice and humans, including the upregulation of RORγt and IL-17A in vitro.

An aspect of the present disclosure provides a method of treating SpA comprising administering a MIF inhibitor to a subject in need thereof.

A further aspect provides use of a MIF inhibitor for treating SpA in a subject in need thereof.

A further aspect provides use of a MIF inhibitor in the manufacture of a medicament for treating SpA in a subject in need thereof.

Also provided is a pharmaceutical composition comprising a MIF inhibitor for treating SpA in a subject in need thereof.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIGS. 1A-1Y are graphs and images that show SKG mice exhibit SpA features with increased expression of MIF and MIF-producing neutrophils: (A and B) Representative histological pictures with hematoxylin and eosin (H&E) staining (A) and pathology scores (B) in PBS- or curdlan-SKG mice (n=10 mice per group) are shown. Arrows indicate inflammation. (C and D) Gene expression of inflammatory markers in SKG ankle soft tissues (n=5 mice per group; C) and splenocytes (n=4 mice per group; D) in response to curdlan is shown. (E) Representative immunofluorescence images show RORγt expression in ankle synovial tissues of PBS- or curdlan-SKG mice. (F) A representative picture of new bone formation (NBF; blue circle) at 8 weeks post-curdlan is shown. (G) Representative histological images with safranin O and fast green staining (SO&FG) show an area of NBF of curdlan-SKG mice at 8 weeks post-curdlan. (H) Gene expression of endochondral ossification markers in ankle tissues of PBS- or curdlan-SKG mice (n=4 mice per group) is shown. (I) Representative microCT images of NBF in PBS- or curdlan-SKG mice at 8 weeks post-curdlan are shown. (J) Weekly concentrations of MIF were measured in serum isolated from PBS- or curdlan-SKG mice (n=5 mice per group). (K) Fold changes in the secretion of MIF are shown for major immune cell lineages (neutrophils, monocytes, B cells and T cells) stimulated with curdlan (1 μg/ml) versus PBS for 60 minutes in vitro (n=5 mice per group). (L) The concentration of secreted MIF into culture media from neutrophils (two million cells per well in a 96 well plate) freshly isolated from healthy controls (HCs) or SpA patients (n=10 per group), with or without stimulation of LPS (0.1 μg/ml for 60 min) or curdlan (1 μg/ml for 60 min), was measured. (M and N) The concentration of secreted MIF into culture media from neutrophils (2 million cells per well in a 96 well plate) freshly isolated from healthy SKG mice was measured. Neutrophils were cultured with or without curdlan [1 μg/ml for 30 minutes (M) or 60 minutes (N)] in the presence or absence of anti-Dectin-1 monoclonal antibodies (mAb) or isotype control IgG2a mAb (n=7 per group). (O) Representative immunoblot images of the expression of phospho-Syk (p-Syk) and Syk and the densitometry of p-Syk adjusted by Syk is shown for SKG neutrophils treated with or without curdlan in the presence or absence of anti-Dectin-1 monoclonal antibodies (mAb) or isotype control IgG2a mAb (n=4 per group). (P and Q) Representative immunohistochemistry images (P) and quantification (Q) of Gr-1-positive cells in PBS- or curdlan-SKG ankle tissues are shown (n=5 mice per group). (R to Y) Proportion and frequency of neutrophils (CD11b⁺CD11c⁻Ly6G⁺Ly6C^{lo}) and monocytes (CD11b⁺CD11c⁻Ly6G⁻Ly6C^{hi}) in ankles or spleens isolated from PBS- or curdlan-SKG mice (n=8 mice per group) are shown. Scale bars in (A, E, G, and P) show 100 μm. Data shown in (B to D, H, J to O, Q, and R to Y) are means ±SEM. Relative and % data was log-transformed prior to statistical analyses. Statistical analyses were performed as follows: Mann Whitney U test (B, R, T, V, and X), two-tailed paired t test (C and K), two-tailed unpaired t test with Welch's correction (H, L, Q, S, U, W, and Y), one-way analysis of variance (ANOVA) with the Geisser-Greenhouse correction followed by two-stage linear step-up procedure of Benjamini, Krieger and Yekutieli post-hoc test (D, and M to O), or Kruskal Wallis test followed by two-stage linear step-up procedure of Benjamini, Krieger and Yekutieli post-hoc test (J). *P (or q)<0.05 and **P (or q)<0.01.

FIGS. 2A-2Q are a series of graphs and images that show MIF-overexpressing SKG mice exhibit clinical features of SpA with associated immunological features: (A) Schematic of hydrodynamic (HDD) delivery system with MIF-plasmid in SKG mice. (B) Representative pictures of clinical features including blepharitis, arthritis (wrist and ankle), and dermatitis (tail) in control plasmid (CTL PLM)- or MIF PLM-injected SKG mice at 8 weeks post-PLM injection. (C and D) Weekly sequential concentration of MIF in the serum of female or male SKG mice injected with CTL PLM or MIF-PLM (n=5 mice/group/sex). (E and F) Weekly clinical scores of body weight, arthritis, blepharitis and dermatitis in female SKG (e) or male SKG mice (f) n=10 mice/group) post-PLM injections. (G) Representative histological images of ankle and sacroiliac joints, tail spine, ileum and skin in CTL PLM- or MIF PLM-injected female SKG mice at 8 weeks post-PLM injections. Arrows point inflammation in the tissues. Scale bars show 100 μm. (H) Histological scores of arthritis (ankle) and spinal inflammation in MIF PLM-injected SKG mice at 0, 5, and 8 weeks post-PLM injection (n=5 mice/group). (I) Representative microCT images of ankle joint in SKG mice at 8 weeks post-CTL PLM or MIF PLM injection (circle and arrow point new bone formation in the distal tibia). (J and K) Frequency of CD4+ T cells expressing IL-17A and IL-22 in popliteal lymph nodes (PLNs) isolated from CTL PLM- or MIF PLM-injected female SKG mice at 8 weeks post-PLM injections (n=5 mice/group). (L and M) Frequency of Th17 cells gated by RORγt+ and/or CCR6+ in CD4+ cells within PLNs from CTL PLM- or MIF PLM-injected female SKG mice at 8 weeks post-PLM injections (n=5 mice/group). (N and O) Frequency of group 2 and 3 innate lymphoid cells (ILC2s and ILC3s) gated by GATA3+ or RORγt+ in lineage marker (CD3, Ly6G/C, CD11b, CD45R/B220 and TER119) negative cell populations isolated from PLNs of CTL PLM- or MIF PLM-injected female SKG mice at 8 weeks post-PLM injection (n=5 mice/group). (P and Q) Frequency of CD25hiFoxp3+ cells in CD4+ cell populations isolated from PLNs of CTL PLM- or MIF PLM-injected female SKG mice at 8 weeks post-PLM injection (n=5 mice/group). Data shown are means ±SEM. Relative and % data was log-transformed prior to statistical analyses. Statistical analyses were performed as follows: Kruskal Wallis test followed by two-stage linear step-up procedure of Benjamini, Krieger and Yekutieli post-hoc test, unpaired two-tailed t test with Welch's correction, or Mann Whitney U test. *P (or q)<0.05 and **P (or q)<0.01.

FIGS. 3A-3Q are graphs and images that show genetic deletion of MIF attenuates curdlan-induced SpA-like clinical features and type 3 immunity in SKG mice. (A) Representative picture of female Mif knockout (KO; Mif−/−) SKG mouse and wild type (WT; Mif+/+) SKG mouse (age: 8 weeks). (B) Comparison of body weight between WT SKG mice and Mif KO SKG mice (n=10 mice/group; age: 8 weeks). (C) Concentration of MIF in the serum of Mif KO, heterozygous (Het; Mif+/−), and WT SKG mice (n=11 mice/group; age: 8 weeks). (D) Weekly clinical scores of body weight, arthritis, dermatitis and blepharitis in Mif KO, Het, or WT SKG mice treated with curdlan over 8 weeks (n=7 mice/group). (E) Representative histological images of ankle joints and tail spines in WT SKG or Mif KO SKG mice at 8 weeks post-curdlan injection. Scale bars show 100 μm. (F and G) Histological scores of arthritis or spinal inflammation between WT SKG and Mif KO SKG mice at 8 weeks post-curdlan injection (n=10 mice/group). (H) Representative microCT images of ankle joint in PBS-injected Mif KO, curdlan-treated SKG, or curdlan-treated Mif KO SKG mice at 8 weeks post-curdlan or PBS treatment. Yellow circle and arrow point new bone formation in the distal tibia of curdlan-treated WT SKG mouse. (I) Expression of inflammatory markers (Il1β, Il6, Il17a, Il23a, Tnfa, and Mcp1) in ankle soft tissues isolated from PBS-treated SKG, curdlan-treated SKG or curdlan-treated Mif KO SKG mice at 8 weeks post-curdlan or PBS treatment (n=4 mice/group). (j) Expression of inflammatory markers (Il1β, Il6, Il17a, Il23a, Tnfa, and Mcp1) in Mif KO SKG-derived splenocytes in response to curdlan treatment (0, 1, 10, 100 μg; n=4 mice/group). (K and L) Frequency of Th17 cells gated by RORγt+ and CCR6+ in CD4+ cell populations isolated from popliteal lymph nodes (PLNs) of PBS-treated SKG (A), curdlan-treated SKG (B), or curdlan-treated Mif KO SKG (C) mice at 8 weeks post-curdlan or PBS treatment (n=6 mice/group). (M) Frequency of IL-17A, IL-22, IFN-γ, or IL-10 expressing CD4+ T cells in PLNs or spleen isolated from WT SKG mice or Mif KO SKG mice at 8 weeks post-curdlan or PBS treatment (n=6 mice/group). (N and O) Frequency of Tregs gated by CD25hi and Foxp3+ in CD4+ cells from PLNs from PBS-treated SKG (A), curdlan-treated SKG (B), or curdlan-treated Mif KO SKG (C) mice at 8 weeks post-curdlan or PBS treatment (n=6 mice/group). (P and Q) Frequency of group 3 innate lymphoid cells (ILC3s) gated by RORγt+ cells in lineage marker (CD3, Ly6G/C, CD11b, CD45R/B220 and TER119) negative cell populations isolated from PLNs of PBS-treated SKG (A), curdlan-treated SKG (B), or curdlan-treated Mif KO SKG (C) mice at 8 weeks post-curdlan or PBS treatment (n=6 mice/group). Data shown are means ±SEM. Relative and % data was log-transformed prior to statistical analyses. Statistical analyses were as follows: Mann Whitney U test, one-way ANOVA with Geisser-Greenhouse correction followed by two-stage linear step-up procedure of Benjamini, Krieger and Yekutieli post-hoc test, Kruskal Wallis test followed by two-stage linear step-up procedure of Benjamini, Krieger and Yekutieli post-hoc test, or Brown-Forsythe and Welch ANOVA test followed by two-stage linear step-up procedure of Benjamini, Krieger and Yekutieli post-hoc test. *P (or q)<0.05 and **P (or q)<0.01.

FIGS. 4A-4T are graphs and images that show prophylactic and therapeutic effects of MIF antagonist (MIF098) on spondyloarthritis (SpA) pathologies in curdlan-treated SKG mice. (A) Schematic image of injection of MIF098 or control vehicle (CTL) into curdlan-treated SKG mice. (B) Representative pictures of clinical features (arthritis, tail dermatitis, and blepharitis) of control female SKG (no antagonist injection), curdlan-treated female SKG mice injected with CTL, curdlan-treated female SKG mice injected with MIF098 (40 mg/kg, i.p. injection, twice daily, from 1 to 8 weeks post-curdlan) at 8 weeks post-curdlan treatment. (C) Weekly clinical scores of body weight, arthritis, dermatitis, and blepharitis in PBS-treated SKG mice (A), curdlan-treated SKG mice injected with CTL (B), or curdlan-treated SKG mice injected with MIF098 (C) over 8 weeks (n=8 mice/group). (D) Representative histological images of ankle, spinal inflammation, sacroiliac joint or ileum of curdlan-treated SKG mice injected with CTL or MIF098 at 8 weeks post-CTL or curdlan treatment. Scale bars show 100 μm. (E) Histological scores of arthritis (ankle) or spinal inflammation of curdlan-treated SKG mice injected with CTL or MIF098 at 8 weeks post-curdlan treatment (n=10 mice/group). (F) Representative microCT images of ankle joint in curdlan-treated SKG mice injected with either CTL or MIF098 at 8 weeks post-curdlan. Yellow arrow points new bone formation (NBF) in the distal tibia of curdlan-treated SKG (Mif+/+) mouse injected with CTL. (G) Representative pictures of NBF in the distal tibia of female SKG mice injected with MIF098 (40 mg/kg, twice/day, i.p., from 1 to 8 weeks post-curdlan) or CTL at 8 weeks post-curdlan treatment. Scale bar indicates 100 μm. (H and I) Frequency of Th17 cells gated by RORγt+ or CCR6+ in CD4+ cell populations isolated from popliteal lymph nodes (PLNs) of curdlan-treated female SKG mice injected with CTL or MIF098 at 8 weeks post-curdlan treatment (n=6 mice/group). (J and K) Frequency of group 3 innate lymphoid cells (ILC3s) gated by RORγt+ in lineage marker (CD3, Ly6G/C, CD11b, CD45R/B220 and TER119) negative cell populations isolated from PLNs of curdlan-treated female SKG mice injected with CTL or MIF098 (n=6 mice/group). (L and M) Frequency of Tregs gated by CD25hi and Foxp3+ in CD4+ cells from PLNs of curdlan-treated female SKG mice injected with CTL or MIF098 at 8 weeks post-curdlan treatment (n=6 mice/group). (N) Schematic image of injection of MIF098 or control vehicle (CTL) into curdlan-treated female SKG mice from 4 weeks to 8 weeks post-curdlan treatment. (O) Representative pictures of arthritis (ankle) or tail dermatitis in curdlan-treated female SKG mice injected with CTL or MIF098 at 8 weeks post-curdlan treatment. (P) Weekly clinical scores of body weight, arthritis, dermatitis or blepharitis over 8 weeks in curdlan-treated female SKG mice injected with CTL or MIF098 (n=5 mice/group). (Q) Representative histological images of ankle and spinal inflammation in curdlan-treated SKG mice injected with CTL or MIF098 at 8 weeks post-curdlan treatment. Scale bars show 100 μm. (R) Histological scores of arthritis or spinal inflammation in curdlan-treated SKG mice injected with CTL or MIF098 at 8 weeks post-curdlan treatment (n=5 mice/group). (S and T) Representative histological pictures of NBF in the distal tibia of female SKG mice injected with MIF098 (from 4 to 8 weeks post-curdlan) or CTL (from 4 to 8 weeks post-curdlan) at 8 weeks post-curdlan treatment, assessed by hematoxylin/eosin (H&E; S) or safranin O/fast green (SO/FG; T) staining. Scale bars indicate 100 μm. Data shown are means ±SEM. % data was log-transformed prior to statistical analyses. Statistical analyses were performed as follows: Kruskal Wallis test followed by two-stage linear step-up procedure of Benjamini, Krieger and Yekutieli post-hoc test, Mann Whitney U test, or two-tailed unpaired t test with Welch's correction. *P (or q)<0.05 and **P (or q)<0.01.

FIGS. 5A-5M are graphs and images that show expansion of MIF-producing neutrophils induces a SpA-like phenotype in SKG mice. (A) Schematic image of adopted transfer (AT) of neutrophils into curdlan-treated female Mif knockout (KO) mice. (B) Weekly clinical scores of body weight, clinical scores of arthritis, blepharitis and dermatitis in curdlan-treated female Mif KO SKG mice transferred with neutrophils from PBS-treated (CTL-neutrophils; A) or curdlan-treated SKG mice (cd-neutrophils; B) over 28 days (n=5 mice/group). (C) Representative pictures of blepharitis (yellow arrow), dermatitis on muzzle (green arrow) and arthritis (wrist; red arrow) in curdlan-treated Mif KO SKG mice at 2 weeks post-AT of either control- neutrophils or cd- neutrophils (2 million cells/injection×2/mouse). (D) Representative histological images of ankle joint or tail spine in curdlan-treated Mif KO SKG mice transferred with control- neutrophils or cd- neutrophils at 2 weeks post-AT. Scale bars show 100 μm. (E) Expression of inflammatory markers in the joint tissues (ankle) at 2 weeks or 8 weeks post-AT of control- neutrophils (A) or cd- neutrophils (B) (n=5 mice/group). (F) Schematic image of anti-Gr-1 (100 μg/mouse) or isotype IgG monoclonal antibody (mAb; 100 μg/mouse) injection into curdlan-treated female SKG mice. (G) Weekly clinical scores of body weight, arthritis, dermatitis and blepharitis in curdlan-treated SKG mice injected with isotype IgG mAb (A) or anti-Gr-1 mAb (B) over 28 days (n=5 mice/group). (H) Representative pictures of ankle joint and tail in curdlan-treated SKG mice injected with anti-Gr-1 or isotype IgG mAb at 15 days post-curdlan treatment. (I) Representative histological images of ankle joint in curdlan-treated SKG mice injected with anti-Gr-1 or isotype IgG mAb at 15 days post-curdlan treatment. Scale bars show 100 μm. (J) Histological scores of arthritis in the ankle joints in curdlan-treated SKG mice injected with isotype IgG or anti-Gr1 mAb at 15 days post-curdlan treatment (n=5 mice/group). (K) Representative pictures of ankle joint and tail in a curdlan-treated SKG mouse injected with anti-Gr-1 or isotype IgG mAb at 28 days post-curdlan treatment. (L) Representative histological images of ankle joint in curdlan-treated SKG mice injected with anti-Gr-1 or isotype IgG mAb at 28 days post-curdlan treatment. Scale bars show 100 μm. (M) Histological scores of arthritis in the ankle joints in curdlan-treated SKG mice injected with isotype IgG or anti-Gr1 mAb at 28 days post-curdlan treatment (n=5 mice/group). Data shown are means ±SEM. Relative data was log-transformed prior to statistical analyses. Statistical analyses were performed as follows: Kruskal Wallis test followed by two-stage linear step-up procedure of Benjamini, Krieger and Yekutieli post-hoc test, two-tailed unpaired t test with Welch's correction, or Mann Whitney U test. *P (or q)<0.05 and **P (or q)<0.01.

FIGS. 6A-6Q are graphs and images that show acquisition of a Th17 cell-like phenotype in regulatory T (Tregs) cells isolated from SKG mice and humans in response to MIF treatment. (A) Frequency of RORγt+Foxp3+ Tregs in CD4+ T cells of SKG mice injected with either MIF-plasmid (MIF PLM) or control-plasmid (CTL PLM) at 8 weeks post-PLM treatment, assessed by flow cytometry (n=5 mice/group). (B) Frequency of RORγt+Foxp3+ Tregs in CD4+ T cells of PBS-treated SKG, curdlan-treated SKG (Mif+/+), or curdlan-treated Mif−/− SKG mice at 8 weeks post-curdlan treatment, assessed by flow cytometry (n=6 mice/group). (C) Frequency of RORγt+Foxp3+ Tregs in CD4+ T cells of curdlan-treated SKG mice injected with either control vehicle (CTL) or MIF antagonist (MIF098) at 8 weeks post-curdlan treatment, assessed by flow cytometry (n=6 mice/group). (I) Schematic image of a Th17 acquisition assay in mouse Tregs stimulated with or without rmMIF treatment for 4 days. (D) Representative data of in vitro suppression assay. Conventional CD4+ T cells (Tconv) isolated from BALB/c mice were cultured with Tregs, isolated from Mif+/+ BALB/c (A), Mif+/+ SKG (B), or Mif−/− SKG (C) mice, at different ratios (Tconv:Treg; 1:2, 1:1, 2:1, 4:1). Data shown were repeated three times with consistent results. (E) Schematic image of a Th17 acquisition assay in mouse Tregs is shown. (F and G) Frequency of RORγt+ Tregs at 4 days post-stimulation with or without recombinant mouse (rm) MIF (50 ng/ml) in vitro is shown (n=5 mice per group). (H and I) Intracellular expression of IL-17A in mouse Tregs cultured with IL-2 (H) and IL-2/IL-1β (I) in the presence or absence of rmMIF is shown (n=5 mice per group). (J) Schematic image of a Th17 acquisition assay in human Tregs stimulated with or without rhMIF treatment (50 ng/ml) for 12 days. (K and L) Frequency of cultured human Tregs, gated by RORγt and Foxp3, at 12 days post-stimulation with either anti-CD3−CD28−CD2+ IL-2 (CTL), CTL+IL-1β, CTL+IL-1β+IL-23, or CTL+IL-1β+IL-23+rhMIF [n=8 /group; two distinct samples per person from n=4 heathy male individuals (18-40 years old)/group]. (M) Heatmap of released cytokines from human Tregs cultured with or without rhMIF (50 ng/ml) in the presence or absence of IL-1β and IL-23 (n=2 samples from 2 healthy control males). Data shown were repeated twice with consistent results. (N to P) Fold changes in the concentration of IL-17A, IL-17F and IL-6 from each CTL, in the culture media of Tregs stimulated with anti-CD3−CD28−CD2+IL-2 (CTL), CTL+IL-1β, CTL+IL-1β+IL-23, or CTL+IL-1β+IL-23+rhMIF, assessed by bead-based multiplex assays [n=4 /group; two distinct samples per person from n=2 heathy male individuals (18-40 years old)/group]. (Q) Concentration of IL-17A in the culture media of human Tregs stimulated with either anti-CD3−CD28−CD2+IL-2 (CTL), CTL+IL-1β, CTL+IL-1β+IL-23, or CTL+IL-1β+IL-23+rhMIF at 12-day post-treatment, assessed by ELISA (n=8 /group; two distinct samples per person from n=4 healthy). Data shown are means ±SEM. Relative and % data was log-transformed prior to statistical analyses. Statistical analyses were performed as follows: two-tailed unpaired t test with Welch's correction, Brown-Forsythe and Welch ANOVA test followed by two-stage linear step-up procedure of Benjamini, Krieger and Yekutieli post-hoc test, two-tailed paired t test, one-way ANOVA with Geisser-Greenhouse correction followed by two-stage linear step-up procedure of Benjamini, Krieger and Yekutieli post-hoc test. *P (or q)<0.05 and **P (or q)<0.01.

FIGS. 7A-7B are images that show increased expression of MIF in human spinal ligament and bone marrow samples from SpA patients. (A) Ligament of spinal process stained with toluidine blue and SO&FG from patients with SpA or OA (disease control) are shown. (B) Representative expression of MIF in spinal bone marrow and spinal ligament obtained from SpA or OA patients, assessed by IHC, are shown (n=6 patients per group). Scale bars in (A and B) show 100 μm.

FIGS. 8A-8B are graphs and images that show intracellular expression of MIF in SKG neutrophils treated with or without curdlan in vitro. (A and B) Representative immunoblotting image (A) and densitometry (B) of MIF expression in cell lysates of SKG neutrophils (two million cells) treated with or without curdlan (1 μg/ml) for 60 minutes (n=5 per group) are shown. Data shown is mean ±SEM. (B) Relative data was log-transformed prior to the statistical analysis. Statistical analysis was performed by paired two-tailed t test. **P<0.01.

FIGS. 9A-9E are graphs and images that show proportions of major immune cell lineages and their MIF production in curdlan-treated SKG mice. (A) Representative proportions of T cells (CD3⁺CD19⁻), B cells (CD19⁺CD3⁻), neutrophils (CD11b⁺Ly6G⁺Ly6C^{lo}) and monocytes (CD11b⁺Ly6G⁻Ly6C^{hi}) isolated from ankle joints of a curdlan-treated SKG mouse are shown. (B) Concentrations of secreted MIF from each cell population (two million neutrophils, 0.22 million monocytes, 0.11 million B cells, and 0.11 million T cells per well) into culture media (HBSS) were measured (n=8 mice per group). (C to E) Frequency of T cells, B cells, neutrophils and monocytes and their expression of MIF in popliteal lymph nodes (PLN) isolated from a PBS- or curdlan-treated SKG mouse is shown. Data shown in (B) are means ±SEM. Statistical analyses were performed by Mann-Whitney U test. *P<0.05 and **P<0.01.

FIGS. 10A-10C are graphs that show the expression of MIF and CD74 in curdlan- or control (PBS)-treated SKG mice. (A) Representative images of MIF expression in spleen of a SKG mouse at 8 weeks post-PBS or curdlan treatment, assessed by IF, are shown. (B) Representative images of MIF and CD74 expression in sacroiliac joints of a SKG mouse at 8 weeks post-PBS or curdlan treatment, assessed by IF, are shown. (C) Representative images of MIF expression in ilea of a SKG mouse at 8 weeks post-PBS or curdlan, assessed by IHC, are shown. Scale bars show 100 μm.

FIGS. 11A-11F are graphs and images that show characteristics of MIF-overexpressing SKG mice. (A) Schematic construct of MIF plasmid (MIF PLM) is shown. (B) Histological images of NBF in the distal tibia of a SKG mouse at 8 weeks post-MIF PLM (H&E or SO&FG staining) are shown. (C) Representative images of IHC for SOX9, type X collagen (COL10A1), MMP13 or isotype negative control in areas of NBF of a SKG mouse at 8 weeks post-MIF PLM injection are shown. Arrows point to positive cells for SOX9. (D) Representative IF images for MIF and CD74 expression along with DAPI in an area of NBF of a SKG mouse at 8 weeks post-MIF PLM injection are shown. (E) Gene expression of chondrogenesis (Sox9), cartilage extracellular matrix (Acan and Col2a1), osteogenesis (Runx2), bone formation (Alpl, Bglap and Bmp2) markers in ankle soft tissues of SKG mice at 8 weeks post-CTL PLM or MIF PLM injection is shown (n=4 mice per group). (F and G) Gene expression of inflammatory markers in ankle soft tissues (F) or spleen (G) of SKG mice at 8 weeks post-control plasmid (CTL PLM) or MIF plasmid (MIF PLM; n=4 mice per group) is shown. Scale bars show 100 μm. Data shown are means ±SEM. Relative data was log-transformed prior to statistical analyses. Statistical analyses were performed by unpaired two-tailed t test with Welch's correction. *P<0.05 and **P<0.01.

FIGS. 12A-12D are images that show the frequency of inflammatory macrophages and patrolling macrophages in curdlan-injected WT SKG or Mif KO SKG mice. (A to D) Frequency of inflammatory macrophages (CD11b+CD11c−Ly6ChiCX3CR1loCCR2+) or patrolling macrophages (CD11b+CD11c−Ly6CloCX3CR1hiCCR2−) in CD11b+ cells in ankle soft tissues of PBS-treated SKG, curdlan-treated SKG, or curdlan-treated Mif KO SKG mice at 8 weeks post-treatment is shown (n=6 mice per group). Statistical analyses were performed by Brown-Forsythe and Welch ANOVA test followed by two-stage linear step-up procedure of Benjamini, Krieger and Yekutieli post-hoc test. **P (or q)<0.01.

FIGS. 13A-13E are images that show the effect of MIF antagonist (MIF098) on curdlan-injected SKG mice and immune cell profiles. (A) Representative histological images (H&E staining) of ankle joint and tail vertebrae in curdlan-injected SKG mice treated with either control (CTL) vehicle or MIF098 at 4 weeks post-curdlan injection are shown. Scale bars show 100 μm. (B to D) Frequency of inflammatory macrophages (CD11b+CD11c−Ly6ChiCX3CR1lo CCR2+) or patrolling macrophages (CD11b+CD11c−Ly6CloCX3CR1hiCCR2−) in CD11b+ cells from ankle soft tissues of curdlan-injected SKG mice treated with CTL or MIF098 at 8 weeks post-curdlan injection is shown (n=6 mice per group). (E) Frequency of Tregs (CD25hiFOXP3+) in CD4+ T cells isolated from spleen of curdlan-injected SKG mice treated with either MIF098 or CTL vehicle at 8 weeks post-curdlan injection is shown (n=6 mice per group). Data shown are means ±SEM. % data was log-transformed prior to statistical analyses. Statistical analyses were performed by unpaired two-tailed t test with Welch's correction. **P<0.01.

FIG. 14A is an image that show representative flow cytometry images in mouse Treg suppression assays. Tregs obtained from wild type (WT; Mif+/+) BALB/c, WT SKG or Mif knockout (Mif−/−; Mif KO) SKG mice were co-cultured with CD4+ T cells obtained from WT BALB/c mice. Numbers at the top of the graph show ratios of Tregs to conventional CD4+ T cells (Tregs:Tconv).

FIGS. 15A-15B are images that show the effect of anti-MIF monoclonal antibody (mAb). (A) Representative histological images of ankle joints and tail vertebrae in curdlan-injected SKG mice treated with anti-MIF mAb (20 mg/kg, every 3 days, i.p. injection) or isotype control IgG1 Ab (20 mg/kg, every 3 days). The anti-MIF or control Ab was administered via intraperitoneal injection from 1 week to 8 weeks post-curdlan injection. (B) Pathological scores for ankle joints and tail vertebrae in curdlan-injected SKG mice treated with anti-MIF or control IgG mAb at 8 weeks post-curdlan injection.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

The term “spondyloarthritis” or SpA as used herein refers to a family of related autoinflammatory diseases, including ankylosing spondylitis (AS) (which is also referred to as radiographic axial spondyloarthritis), non-radiographic axial SpA (nr-axSpA), reactive arthritis (ReA), psoriatic arthritis (PsA), IBD-related SpA, juvenile-onset idiopathic arthritis (JIA) and undifferentiated SpA (USpA). Broadly SpA may be divided into axial and peripheral SpA (AxSpA and perSpA) based on the predominant manifestations being back pain or peripheral joint symptoms respectively. Patients with SpA can have a variety of symptoms such as lower back pain, joint inflammation and/or radiologic findings such as inflammation and evidence of NBF or fusion. Patients can be in remission, be experiencing at least one SpA articular or extra-articular (e.g. inflammatory bowel disease (IBD) (e.g. ulcerative colitis or Crohn's diseases), psoriasis, iritis, dermatitis, or uveitis) symptom and/or have periods of flares.

The term “ankylosing spondylitis” or “AS” also referred to as “radiographic axial spondyloarthritis” as used herein, refers to a disease featured by chronic inflammatory arthritis primarily affecting the axial joints typically including involvement of the sacroiliac (SI) joints and in severe cases leading to vertebral fusion. Extra-articular symptoms can include one or more of uveitis or iritis which is present in about 20-40% of AS patients, psoriasis/dermatitis, and inflammatory bowel disease.

The term “early SpA” as used herein refers to a disease stage prior to radiographic findings appearing on X-rays in the sacroiliac joints (SIJs) that fulfill the modified New York (mNY) criteria for AS. Early SpA can include chronic back pain for example lasting less than 1 year, with MRI evidence of inflammation, but no X-ray changes, in the spine. HLA-B27 is a gene seen in 80% of patients with AS.

The term “late SpA” as used herein refers to a disease stage after new bone formation (NBF) results in extensive spinal fusion (at least two adjacent vertebrae fused) and/or more than grade 3 SIJs changes according to mNY criteria, assessed by X-rays

The term “extra-articular symptom” as used herein refers to a symptom or associated condition with SpA or a subtype thereof. Examples include uveitis or iritis, psoriasis, and/or inflammatory bowel disease.

The term “MIF inhibitor” includes any molecule that binds MIF (macrophage migration inhibitory factor), particularly human MIF, binds CD74, particularly human CD74, inhibits MIF-CD74 signal transduction (e.g. by inhibiting, or interfering with MIF-CD74 receptor binding), particularly human MIF-CD74 signal transduction, and/or inhibits MIF tautomerase activity, including for example MIF inhibitors described in WO2010021693 and U.S. Pat. No. 9,643,922 (MIF Modulators), each of which are herein incorporated by reference, Ibudilast (MN166), 2-methyl-1-(2-propan-2-ylpyrazolo[1,5-a]pyridin-3-yl)propan-1-one), CPSI-2705, CPSI-1306 (US20050250826; PCT/US11/21721 or national phase entry U.S. application Ser. No. 13/574,240, each of which are incorporated by reference in their entirety, Cytokine Pharmasciences), isoxazoline (ISO-1) (TalBiochem) as well as an anti-MIF antibody or a binding fragment thereof, such as the anti-MIF monoclonal antibody described in (Leng et al., J Immunol 186, 527-38 (2011)) herein incorporated by reference or imalumab (Takeda Pharmaceuticals), or an anti-CD74 antibody or binding fragment thereof, that for example inhibits CD74 and MIF binding or inhibits CD74-MIF signalling, for example anti-CD74 humanized monoclonal antibody Milatuzumab. The MIF inhibitor can for example bind MIF and/or CD74 and inhibit MIF-CD74 signal transduction. MIF-CD74 signal transduction can be assessed for example using the assay described in MIF- signal transduction initiated by binding to CD74 (Leng et al., J Exp Med 197,1467-1476 (2003)) (Ranganathan et al., Arthritis Rheumatol 69, 1796-1806 (2017)). MIF tautomerase activity can be assessed in a tautomerase assay that monitors the keto/enol interconversion for p-hydroxyphenylpyruvate (HPP) catalyzed by MIF (Stamps, S. L., (2000), Mechanism of the Phenylpyruvate Tautomerase Activity of Macrophage Migration Inhibitory Factor Properties of the P1G, P1A, Y95F, and N97A Mutants Biochemistry 39, 9671-9678). The level of signal transduction or tautomerase activity inhibition can for example be at least 60%, at least 70%, at least 80% or at least 90% compared to vehicle. Other examples of MIF inhibitors include, e.g., U.S. Pat. No. 6,774,227, Bernhagen et al., Nature 365, 756-759 (1993), Senter et al., Proc Natl Acad Sci USA 99:144-149 (2002); Dios et al., J. Med. Chem. 45:2410-2416 (2002); Lubetsky et al., J Biol Chem 277:24976-24982 (2002), which are hereby incorporated by reference. Although inhibition of tautomerase enzymatic activity is not linked to inhibiting MIF-CD74 interaction, likely due to the proximity of this site to the MIF-CD74 interaction, those inhibitors that bind to the tautomerase site can effectively inhibit CD74 mediated MIF action (Kok et al., Drug Discov Today 23, 1910-1918 (2018)). The MIF inhibitors contemplated are for example, directed to interrupting extracellular MIF activation of CD74.

For example, the MIF inhibitor may comprise a compound of Formula I in WO2010021693 and U.S. Pat. No. 9,643,922 (MIF Modulators) that inhibits MIF as described herein , e.g. inhibits MIF-CD74 signal transduction, and/or inhibits MIF tautomerase activity such compound having a chemical structure of (I):

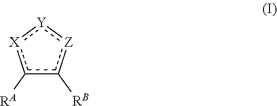

where X is O, S, N—R^{XN1} or CR^{XC1}R^{XC2};
Y is N—R^{YN1} or CR^{YC1}R^{YC2}; and
Z is O, S, N—R^{ZN1} or CR^{ZC1}R^{ZC2}, with the proviso that at least one of X or Z is N—R^{YN1} and X and Z are other than O, when Y is O;
R^{XN1} is absent (N is —N═, thus forming a double bond with an adjacent atom), H or an optionally substituted C₁-C₈ alkyl, alkene or alkyne group, an optionally substituted C₁-C₇ acyl group, an optionally substituted (CH₂)j-phenyl group or an optionally substituted (CH₂)ₘ-heterocyclic (preferably heteroaryl) group, or an optionally substituted carbonyl phenyl group, or an optionally substituted carbonyl heteroaryl group;
R^{YN1} is absent, H, an optionally substituted C₁-C₈ alkyl, alkene or alkyne group, an optionally substituted C₁-C₈ acyl group, an optionally substituted (CH₂)j-phenyl group or an optionally substituted (CH₂)m-heterocyclic (preferably heteroaryl) group, or an optionally substituted carbonyl phenyl group, or an optionally substituted carbonyl heteroaryl group;
R^{ZN1} is absent, H, an optionally substituted C₁-C₈ alkyl, alkene or alkyne group, an optionally substituted C₁-C₈ acyl group, an optionally substituted (CH₂)j-phenyl group or an optionally substituted (CH₂)m-heterocyclic (preferably heteroaryl) group, or an optionally substituted carbonyl phenyl group, or an optionally substituted carbonyl heteroaryl group;
R^{XC1} is absent (C is —C═, thus forming a double bond with an adjacent atom), H, an optionally substituted C₁-C₃ alkyl, or together with R^{XC2} forms a ═O (keto) or ═C group, (preferably R^{XC1} is absent);
R^{XC2} is H, halogen, cyano, an optionally substituted C₁-C₈ alkyl, alkene or alkyne group (preferably R^{XC2} is an optionally substituted C₁-C₃ group when R^{XC1} is an optionally substituted C₁-C₃ group), an optionally substituted C₁-C₈ acyl group, an optionally substituted C₂-C₈ ester (hydroxyester) or carboxyester group, an optionally substituted C₁-C₇ alkoxy group, an optionally substituted C₂-C₈ ether group, an optionally substituted C₁-C₇ amido or carboxamido group, a C₁-C₇ urethane or urea group, an optionally substituted (CH₂)j-phenyl group or an optionally substituted (CH₂)m-heterocyclic (preferably heteroaryl) group, or together with R^{XC1} forms a ═O (keto) or ═C group, which is optionally substituted with a C₁-C₆ alkyl group, an optionally substituted (CH₂)j-phenyl group or an optionally substituted (CH₂)m-heterocyclic (preferably heteroaryl) group;
R^{YC1} is absent, H, an optionally substituted C₁-C₈ alkyl, or together with R^{YC2} forms a ═O (keto) or ═C which is optionally substituted with a heterocyclic group;
R^{YC2} is H, halogen, cyano, an optionally substituted C₁-C₈ alkyl, alkene or alkyne group (preferably R^{YC2} is an optionally substituted C₁-C₃ group when R^{YC1} is an optionally substituted C₁-C₃ group), an optionally substituted C₁-C₇ acyl group, an optionally substituted C₂-C₈ ester or carboxyester group, an optionally substituted C₁-C₁₀ alkoxy group, an optionally substituted C₂-C₈ ether group, an optionally substituted C₁-C₇ amido or carboxamido group, a C₁-C₇ urethane or urea group, an optionally substituted (CH₂)j-phenyl group or an optionally substituted (CH₂)m-heterocyclic (preferably heteroaryl) group, or together with R^{YC1} forms a ═O (keto) or ═C group, which is optionally substituted with a C₁-C₆ alkyl group, an optionally substituted (CH₂)j-phenyl group or an optionally substituted (CH₂)m-heterocyclic (preferably heteroaryl) group;
R^{ZC1} is absent, H, an optionally substituted C₁-C₃ alkyl, or together with R^{ZC2} forms a ═O (keto) group or a ═C group, (preferably R^{ZC1} is absent);
R^{ZC2} is H, halogen, cyano, an optionally substituted C₁-C₈ alkyl, alkene or alkyne group (preferably R^{ZC2} is an optionally substituted C₁-C₃ group when R^{ZC1} is an optionally substituted C₁-C₃ group), an optionally substituted C₁-C₈ acyl group, an optionally substituted C₂-C₈ ester or carboxyester group, an optionally substituted C₁-C₇ alkoxy group, an optionally substituted C₂-C₈ ether group, an optionally substituted C₁-C₇ amido or carboxamido group, a C₁-C₇ urethane or urea group, an optionally substituted (CH₂)j-phenyl group or an optionally substituted (CH₂)m-heterocyclic (preferably heteroaryl) group, or together with R^{ZC1} forms a ═O (keto) or ═C group, which is optionally substituted with a C₁-C₆ alkyl group, an optionally substituted (CH₂)j-phenyl group or an optionally substituted (CH₂)m-heterocyclic (preferably heteroaryl) group;
R^{A} and R^{B} together form an optionally substituted 5, 6 or 7 membered carbocyclic or heterocyclic ring (preferably an optionally substituted 6-membered aromatic or heteroaromatic ring, more preferably an optionally substituted phenyl ring or a heteroaromatic ring containing one nitrogen group, preferably a pyridyl group);
each j is independently 0, 1, 2, 3, 4 or 5; and
each m is 0, 1, 2, 3, 4, or 5.

For example, the MIF inhibitor may comprise a compound of Formula II in WO2010021693 and U.S. Pat. No. 9,643,922 (MIF Modulators) that inhibits MIF as described herein, e.g. inhibits MIF-CD74 signal transduction, and/or inhibits MIF tautomerase activity, such compound having a chemical structure of (II):

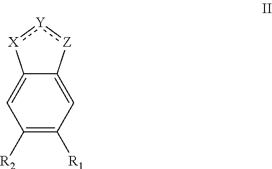

where X, Y Z are as described above for compound (I); and
R₁ and R₂ are each independently H, OH, COOH, halogen (F, Cl, Br, I), CN, OH, optionally substituted C₁-C₈ alkyl, optionally substituted O—(C₁-C₆)alkyl, SH, S—(C₁-C₆)alkyl, optionally substituted C₁-C₈ acyl, optionally substituted C₂-C₈ ether, optionally substituted C₂-C₈ ester or carboxyester, optionally substituted C₂-C₈ thioester, amide optionally substituted with a C₁-C₆ alkyl group, carboxyamide optionally substituted with one or two C₁-C₆ alkyl or alkanol groups, and amine optionally substituted with one or two C₁-C₆ alkyl or alkanol groups. Preferably R₁ and R₂ are independently H, CH₃, CH₂CH₃, NH₂, NHCH₃, N(CH₃)₂, OH, OCH3, SH, SCH₃, F, Cl, Br or I.

For example, the MIF inhibitor may comprise a compound of Formula IIA in WO2010021693 and U.S. Pat. No. 9,643,922 (MIF Modulators) that inhibits MIF as described herein, e.g. inhibits MIF-CD74 signal transduction, and/or inhibits MIF tautomerase activity, such compound having a chemical structure of (IIA):

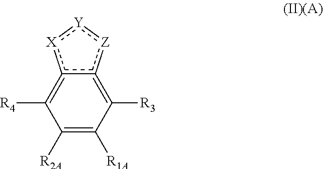

wherein X is O, S, N—R^{XN1} or CR^{XC1}R^{XC2};
Y is N—R^{YN1} or CR^{YC1}R^{YC2};
Z is O, S, N—R^{ZN1} or CR^{ZC1}R^{ZC2}, with the proviso that one of X, Y, or Z is, respectively, CR^{XC1}R^{XC2}, CR^{YC1}R^{YC2}, or CR^{ZC1}R^{ZC2};
R^{XN1} is absent (N is —N═, thus forming a double bond with an adjacent atom), H or an optionally substituted C₁-C₈ alkyl, alkene or alkyne group, an optionally substituted C₁-C₇ acyl group, an optionally substituted (CH₂)j-phenyl group or an optionally substituted (CH₂)ₘ-heterocyclic (preferably heteroaryl) group, or an optionally substituted carbonyl phenyl or an optionally substituted carbonyl heteroaryl group;
R^{YN1} is absent, H, an optionally substituted C₁-C₈ alkyl, alkene or alkyne group, an optionally substituted C₁-C₈ acyl group, an optionally substituted (CH₂)j-phenyl group or an optionally substituted (CH₂)m-heterocyclic (preferably heteroaryl) group, or an optionally substituted carbonyl phenyl or an optionally substituted carbonyl heteroaryl group;
R^{ZN1} is absent, H, an optionally substituted C₁-C₈ alkyl, alkene or alkyne group, an optionally substituted C₁-C₈ acyl group, an optionally substituted (CH₂)j-phenyl group or an optionally substituted (CH₂)m-heterocyclic (preferably heteroaryl) group, or an optionally substituted carbonyl phenyl or an optionally substituted carbonyl heteroaryl group;
R^{XC1} is absent (C is —C═, thus forming a double bond with an adjacent atom), H, an optionally substituted C₁-C₃ alkyl, or together with R^{XC2} forms a ═O (keto) or ═C group, (preferably R^{XC1} is absent);
R^{XC2} is H, halogen, cyano, an optionally substituted C₁-C₈ alkyl, alkene or alkyne group (preferably R^{XC2} is an optionally substituted C₁-C₃ group when R^{XC1} is an optionally substituted C₁-C₃ group), an optionally substituted C₁-C₈ acyl group, an optionally substituted C₂-C₈ ester (hydroxyester) or carboxyester group, an optionally substituted C₁-C₇ alkoxy group, an optionally substituted C₂-C₈ ether group, an optionally substituted C₁-C₇ amido or carboxamido group, a C₁-C₇ urethane or urea group, an optionally substituted (CH₂)j-phenyl group or an optionally substituted (CH₂)m-heterocyclic (preferably heteroaryl) group, or together with R^{XC1} forms a ═O (keto) or ═C group, which is optionally substituted with a C₁-C₆ alkyl group, an optionally substituted (CH₂)j-phenyl group or an optionally substituted (CH₂)m-heterocyclic (preferably heteroaryl) group, or an optionally substituted carbonyl phenyl or an optionally substituted carbonyl heteroaryl group;
R^{YC1} is absent, H, an optionally substituted C₁-C₃ alkyl, or together with R^{YC2} forms a ═O (keto) or ═C which is optionally substituted with a heterocyclic group;

R^{YC2} is H, halogen, cyano, an optionally substituted C₁-C₈ alkyl, alkene or alkyne group (preferably R^{YC2} is an optionally substituted C₁-C₃ group when R^{YC1} is an optionally substituted C₁-C₃ group), an optionally substituted C₁-C₇ acyl group, an optionally substituted C₂-C₈ ester or carboxyester group, an optionally substituted C₁-C₁₀ alkoxy group, an optionally substituted C₂-C₈ ether group, an optionally substituted C₁-C₇ amido or carboxamido group, a C₁-C₇ urethane or urea group, an optionally substituted (CH₂)j-phenyl group or an optionally substituted (CH₂)m-heterocyclic (preferably heteroaryl) group, or together with R^{YC1} forms a ═O (keto) or ═C group, which is optionally substituted with a C₁-C₆ alkyl group, an optionally substituted (CH₂)j-phenyl group or an optionally substituted (CH₂)m-heterocyclic (preferably heteroaryl) group, or an optionally substituted carbonyl phenyl or an optionally substituted carbonyl heteroaryl group;
provided that when R^{XC1} and R^{YC1} are absent, R^{XC2} and R^{YC2} can together form an optionally substituted 5, 6 or 7 membered carbocyclic or heterocyclic ring (preferably an optionally substituted 6-membered aromatic or heteroaromatic ring, more preferably an optionally substituted phenyl ring or a heteroaromatic ring);
R^{ZC1} is absent, H, an optionally substituted C₁-C₃ alkyl, or together with R^{ZC2} forms a ═O (keto) group or a —C group, (preferably R^{ZC1} is absent);
R^{ZC2} is H, halogen, cyano, an optionally substituted C₁-C₈ alkyl, alkene or alkyne group (preferably R^{ZC2} is an optionally substituted C₁-C₃ group when R^{ZC1} is an optionally substituted C₁-C₃ group), an optionally substituted C₁-C₈ acyl group, an optionally substituted C₂-C₈ ester or carboxyester group, an optionally substituted C₁-C₇ alkoxy group, an optionally substituted C₂-C₈ ether group, an optionally substituted C₁-C₇ amido or carboxamido group, a C₁-C₇ urethane or urea group, an optionally substituted (CH₂)j-phenyl group or an optionally substituted (CH₂)m-heterocyclic (preferably heteroaryl) group, or together with R^{ZC1} forms a ═O (keto) or ═C group, which is optionally substituted with a C₁-C₆ alkyl group, an optionally substituted (CH₂)j-phenyl group or an optionally substituted (CH₂)ₘ-heterocyclic (preferably heteroaryl) group, or an optionally substituted carbonyl phenyl group or an optionally substituted carbonyl heteroaryl group;
each j is independently 0, 1, 2, 3, 4 or 5;
each m is 0, 1, 2, 3, 4, or 5;
R_{1A}, R_{2A} R₃, and R₄ are the same or different and are each independently H, OH, COOH, halogen (F, Cl, Br, I), CN, OH, an optionally substituted C₁-C₈ alkyl, alkene or alkyne group, optionally substituted alkyl, alkene or alkyne) group, SH, S—(C₁-C₆)alkyl, optionally substituted C₁-C₈ acyl, optionally substituted C₂-C₈ ether, optionally substituted C₂-C₈ ester or carboxyester, optionally substituted C₂-C₈ thioester, amide optionally substituted with a C₁-C₆ alkyl group, carboxyamide optionally substituted with one or two C₁-C₆ alkyl or alkanol groups, amine optionally substituted with one or two C₁-C₆ alkyl or alkanol groups, an optionally substituted (CH₂)j-phenyl group, an optionally substituted (CH₂)ₘ-heterocyclic (preferably heteroaryl) group, an optionally substituted —O—(CH₂)j-phenyl group or an optionally substituted —O—(CH₂)ₘ-heterocyclic (preferably heteroaryl) group, or an optionally substituted carbonyl phenyl or an optionally substituted carbonyl heteroaryl group.

As a further example, the MIF inhibitor may comprise a compound of Formula B in U.S. Pat. No. 9,643,922 that inhibits MIF as described herein, e.g. inhibits MIF-CD74 signal transduction, and/or inhibits MIF tautomerase activity, such compound having a chemical structure of B:

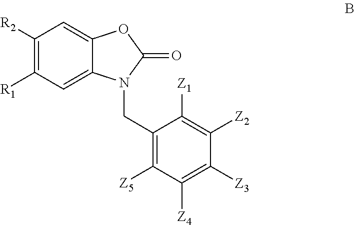

or a pharmaceutically acceptable salt thereof, wherein:
R₁ is hydroxyl, optionally substituted C₁-C₈ alkyl, optionally substituted C₁-C₁₀ alkoxy, F, Cl, or (CH₂)ⱼ—OH; and R² is H; or R₁ is H, and R₂ is hydroxyl, optionally substituted C₁-C₈ alkyl, optionally substituted C₁-C₁₀ alkoxy, F, Cl, or (CH₂)ⱼ—OH; Z₁ is hydroxyl, optionally substituted C₁-C₈ alkyl, C₁ alkoxy, F, Cl, or (CH₂)ⱼ—OH; Z₂ is H; and Z₃ is H; or Z₁ is H; Z₂ is hydroxyl, optionally substituted C₁-C₈ alkyl, optionally substituted C₁-C₁₀ alkoxy, F, Cl, or (CH₂)ⱼ—OH; and Z₃ is H; or Z₁ is H; Z₂ is H; and Z₃ is hydroxyl, optionally substituted C₁-C₈ alkyl, optionally substituted C₁-C₁₀ alkoxy, F, Cl, or (CH₂)ⱼ—OH; Z₄ is H; Z₅ is H; and each j is independently 0, 1, 2, 3, 4 or 5.

For example, the compound according formula B can have a chemical structure of:

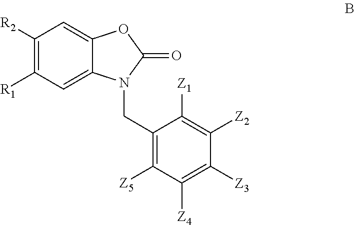

or a pharmaceutically acceptable salt thereof, wherein
R₁ is selected from OH, OCH₃, F, Cl, C₁-C₄ alkyl which is optionally substituted with from one to three hydroxyl groups or from one to three halogen groups, and —(CH₂)ⱼOR^{a}; and R₂ is H; or R₁ is H and R₂ is selected from OH, F, Cl, C₁-C₄ alkyl which is optionally substituted with from one to three hydroxyl groups or from one to three halogen groups, and —(CH₂)ⱼOR^{a}; Z₁ is selected from OCH₃, a C₁-C₃ alkyl group which is optionally substituted with from one to three one hydroxyl groups or from one to three halogen groups, and —(CH₂)ⱼOR^{a}; Z₂ is H; and Z₃ is H; or Z₁ is H; Z₂ is selected from OH, OCH₃, a C₁-C₃ alkyl group which is optionally substituted with from one to three one hydroxyl groups or from one to three halogen groups, and —(CH₂)ⱼOR^{a}; and Z₃ is H; or Z₁ is H; Z₂ is H; and Z₃ is selected from OCH₃, a C₁-C₃ alkyl group which is optionally substituted with from one to three one hydroxyl groups or from one to three halogen groups, and —(CH₂)ⱼOR^{a}; Z₄ is H; Z₅ is H; each R^{a} is independently H, or a methyl or ethyl group which is optionally substituted with a hydroxyl group or from one to three halogen groups; and each j is independently 0, 1, 2, or 3.

As another example, the compound of formula B can have a chemical structure of:

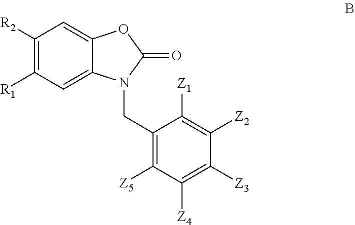

or a pharmaceutically acceptable salt thereof, wherein R₁ is CH₃, OCH₃, F, or OH; R₂ is H, CH₃ or OH; Z₁ is H or OCH₃; Z₂ is H or OH; Z₃ is H or OCH₃; Z₄ is H; and Z₅ is H; or R, is CH₃, OCH₃, F, or OH; R₂ is H, CH₃ or OH; Z₁ is H or OCH₃; Z₂ is H, OH or OCH₃; Z₃ is H; Z₄ is H; and Z₅ is H; and wherein Z₁ is OCH₃; or Z₂ is OH or OCH₃; or; Z₃ is OCH₃.

As a further example, the MIF inhibitor can comprise a compound selected from the following compounds and pharmaceutically acceptable salts thereof: 3-benzyl-5-fluorobenzoxazol-2-one; 3-(2-benzyloxybenzyl)-5-methylbenzoxazol-2-one; 3-(3-cyanobenzyl)-5-chlorobenzoxazol-2-one; 3-(2,3-dimethoxybenzyl)-5-hydroxybenzoxazol-2-one; 3-(2,3-dimethoxybenzyl)-5-methylbenzoxazol-2-one; 3-(2-ethoxybenzyl)-5-methylbenzoxazol-2-one; 3-(3,5-dimethoxybenzyl)-5-methylbenzoxazol-2-one; 3-(3-ethoxybenzyl)-5-methylbenzoxazol-2-one; 3-(3-ethoxy-5-hydroxybenzyl)-5-methylbenzoxazol-2-one; 5-ethyl-3-(3-hydroxybenzyl)benzoxazol-2-one; 5-ethyl-3-(3-methoxybenzyl)benzoxazol-2-one; 3-(3-fluorobenzyl)-5-methylbenzoxazol-2-one; 3-(4-fluorobenzyl)-5-methylbenzoxazol-2-one; 5-fluoro-3-(3-hydroxybenzyl)benzoxazol-2-one; 6-fluoro-3-(3-hydroxybenzyl)benzoxazol-2-one; 5-fluoro-3-(2-methoxybenzyl)benzoxazol-2-one; 5-fluoro-3-(3-methoxybenzyl)benzoxazol-2-one; 5-fluoro-3-(4-methoxybenzyl)benzoxazol-2-one; 6-fluoro-3-(3-methoxybenzyl)benzoxazol-2-one; 3-(3-hydroxybenzyl)-5-methoxybenzoxazol-2-one; 3-(3-hydroxybenzyl)-5-methylbenzoxazol-2-one; 3-(3-hydroxybenzyl)-6-methylbenzoxazol-2-one; 3-(4-hydroxybenzyl)-5-methylbenzoxazol-2-one; 5-hydroxy-3-(3-hydroxybenzyl)benzoxazol-2-one; 5-hydroxy-3-(2-methoxybenzyl)benzoxazol-2-one; 5-hydroxy-3-(3-methoxybenzyl)benzoxazol-2-one; 6-hydroxy-3-(2-methoxybenzyl)benzoxazol-2-one; 6-hydroxy-3-(4-methoxybenzyl)benzoxazol-2-one; 5-(hydroxymethyl)-3-(3-methoxybenzyl)benzoxazol-2-one; 3-(2-methoxybenzyl)-5-methylbenzoxazol-2-one; 3-(3-methoxybenzyl)-5-methylbenzoxazol-2-one; 3-(3-methoxybenzyl)-6-methylbenzoxazol-2-one; 3-(3-methoxybenzyl)-5,6-dimethylbenzoxazol-2-one; and 5-methoxy-3-(3-methoxybenzyl)benzoxazol-2-one. The compounds as described in WO2010021693 and U.S. Pat. No. 9,643,922 (MIF Modulators) can be prepared as described therein.

The term “MIF098” as used herein refers to the compound

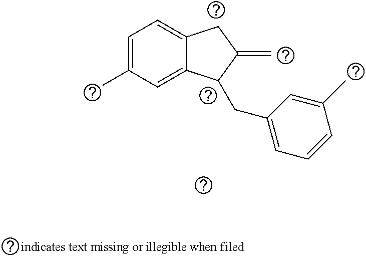

or a pharmaceutically acceptable salt, enantiomer, solvate or polymorph thereof for example as described in U.S. Pat. No. 9,643,922. Methods of making are described therein.

The term MIF098 analog as used herein includes any one of

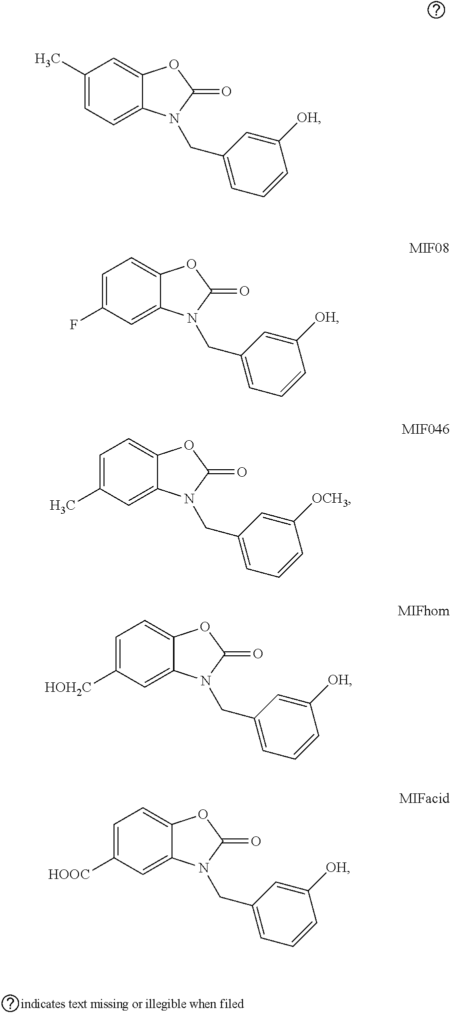 [SMILES: Cc1ccc2c(c1)oc(=O)n2Cc1cccc(O)c1 ; O=c1oc2ccc(F)cc2n1Cc1cccc(O)c1 ; COc1cccc(Cn2c(=O)oc3ccc(C)cc32)c1 ; O=c1oc2ccc(CO)cc2n1Cc1cccc(O)c1 ; O=C(O)c1ccc2oc(=O)n(Cc3cccc(O)c3)c2c1]

or a pharmaceutically acceptable salt, enantiomer, solvate or polymorph thereof or combinations thereof as described for example in U.S. Pat. No. 9,643,922. Methods of making are described therein.

The term “treatment” or “treating” as used herein means administering to a subject a therapeutically effective amount of a compound or composition, and may consist of a single administration, or alternatively comprise a series of administrations. As well understood in the art, “treatment” or “treating” is an approach for obtaining beneficial or desired results, including clinical results. Beneficial or desired clinical results can include, but are not limited to alleviation or amelioration of one or more symptoms or conditions, diminished extent of disease, stabilized (i.e. not worsening) state of disease, preventing spread of disease, reversal of disease, amelioration or palliation of the disease state, and remission (whether partial or total and optionally temporary). Beneficial or desired clinical results can also include reduction in frequency or intensity of flares. Treatment may result in stabilization of disease, an improvement in disease status, or normalization of ongoing inflammation. For example, treatment response can be the improvement or resolution of one or more disease features, including but not limited to inflammatory back pain, SIJ inflammation, and/or decreased flare up frequency or intensity of pain or inflammation in eyes, gut or skin. It can also include improvement in an articular or extra-articular symptom.

As used herein, “contemporaneous administration” and “administered contemporaneously” means for example in reference to two substances (e.g. two compounds, two compositions etc.) that the two substances are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. In particular embodiments, the substances (e.g. two or more compounds or compositions etc.) will be administered substantially simultaneously, i.e. within minutes of each other, or in a single composition that comprises both substances.

As used herein, the phrase “effective amount” or “therapeutically effective amount” means an amount effective, at dosages and for periods of time necessary to achieve a desired result.

The term “flares” as used herein refers to clinical exacerbations of clinical disease activity usually involving increase in symptoms and signs. Flares are typically followed by temporary periods of remission when symptoms subside.

The term “antibody” as used herein is intended to include monoclonal antibodies including chimeric and humanized monoclonal antibodies, polyclonal antibodies, humanized antibodies, human antibodies, and chimeric antibodies. Single chain antibodies are also contemplated. The antibody may be from recombinant sources and/or produced in transgenic animals. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

Additional examples of antigen-binding fragments include an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

The term “composition” as used herein, refers to a mixture comprising two or more compounds or components. For example, composition is a composition of two or more distinct compounds. In a further embodiment, a composition can comprise two or more “forms” of the compounds, such as, salts, solvates, or, where applicable, stereoisomers of the compound in any ratio. A person of skill in the art would understand that a compound in a composition can also exist as a mixture of forms. For example, a compound may exist as a hydrate of a salt. All forms of the compounds disclosed herein are within the scope of the present disclosure.

The term “subject” also referred as patient, as used herein includes all members of the animal kingdom including mammals, and suitably refers to humans.

The terms “pharmaceutically acceptable carrier,” “pharmaceutically acceptable diluent” and “pharmaceutically acceptable excipient” include any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various adjuvants such as are commonly used in the art may be included. These and other such therapeutic agents are described in the literature, e.g., in the Merck Index, Merck & Company, Rahway, N.J. Considerations for the inclusion of various components in pharmaceutical formulations are described, e.g., in Gilman et al. (Eds.) (2010); Goodman and Gilman's: The Pharmacological Basis of Therapeutics, 12th Ed., The McGraw-Hill Companies.

As used herein, a reference to a drug's international nonproprietary name (INN) is to be interpreted as including generic, bioequivalent and biosimilar versions of that drug, including but not limited to any drug that has received abbreviated regulatory approval by reference to an earlier regulatory approval of that drug. Additionally, all drugs disclosed herein optionally include the pharmaceutically acceptable salts and solvates of the drugs thereof, unless expressly indicated otherwise.

The term “compound” as used herein, unless otherwise indicated, refers to any specific chemical compound disclosed herein and includes tautomers, regioisomers, geometric isomers as applicable , and also where applicable, optical isomers (e.g. enantiomers) thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be under-stood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Although any methods and materials similar or equivalent to those described herein can also be used in the practice or testing of the present disclosure, examples of methods and materials are now described.

### II. Methods, Uses and Compositions for Use

Type 3 immunity-mediated inflammatory arthritis, represented by spondyloarthritis (SpA), is a systemic rheumatic disease that primarily affects the joints, spine, gut, skin and eyes.

Traditionally there were only a limited number of treatments available for spondyloarthritis (SpA) and the available treatments did not resolve extra-articular symptoms of SpA.

Macrophage migration inhibitory factor (MIF) is an immune-regulatory cytokine. As demonstrated below, the expression of MIF and its receptor CD74 are increased in blood, spleen, gut, sacroiliac and ankle joints of curdlan-treated SKG mice, a mouse model of SpA. It is further shown that delivery of a MIF-enhanced episomal vector EEV in vivo to overexpress MIF is sufficient to induce SpA-like clinical manifestations in SKG mice including expanded populations of T helper 17 (Th17) cells, group 3 innate lymphoid cells and inflammatory macrophages, with decreased regulatory T cells (Tregs) in the inflamed joints. In contrast, Mif-knockout (Mif KO) SKG mice and SKG mice treated with a MIF antagonist prevent or attenuate these manifestations with substantial reduction of type 3 immunity. Further, neutrophils are demonstrated to expand and produce MIF in the disease.

As used herein, PMN-MDSCs and neutrophils are used interchangeably, and mMDSCs and monocytes are used interchangeably.

Cell adoptive transplantation of neutrophils into Mif KO SKG mice induces a SpA-like phenotype, while blocking the function of neutrophils with anti-Gr-1 antibody suppresses the induced SpA-like phenotype. Without wishing to be bound by theory, mechanistically, MIF enhances acquisition of a Th17 cell-like phenotype and suppresses expansion of Tregs from naïve CD4+ T cells. It is also demonstrated that MIF boosts both human and mouse Treg acquisition of a Th17 cell-like phenotype, including the upregulation of RORγt and IL-17A in vitro. These results indicate that MIF is a crucial regulator of type 3 immunity-mediated inflammation and therapeutic target in SpA.

Accordingly, provided herein are methods, compositions and uses for treating SpA.

An aspect is directed to a method of treating SpA in a subject comprising administering a MIF inhibitor to a subject in need thereof.

In one embodiment the SpA is early SpA. Patients can be administered the MIF inhibitor upon diagnosis. As demonstrated in the examples, the MIF inhibitors provided were able to inhibit progression of ankylosing spondylitis (AS) before radiologic changes were detectable.

In another embodiment, the SpA is late SpA. As demonstrated in the Examples, the MIF inhibitors were also able to inhibit late stage radiologic changes when joint damage was visible.

The subject may comprise one or more symptoms associated with SpA, optionally one or more articular or extra-articular symptoms. In one embodiment, the subject is treated during a flare. In another embodiment, the subject is treated while in remission. For example, the subject may be treated when one or markers suggest that inflammation is worsening such as CRP (C-reactive protein) or ESR (erythrocyte sedimentation rate). Alternatively, the subject may have increased pain or other symptom of SpA without elevation of CRP and/or ESR.

In one embodiment, the SpA is ankylosing spondylitis.

In another embodiment, the subject is a patient with a higher likelihood of progression (e.g. those with elevated inflammatory parameters ESR/CRP, baseline existing NBF and/or smokers).

Remarkably and as demonstrated in the Examples, the MIF inhibitors also resolved extra-articular symptoms.

Also provided is a method of inhibiting new bone formation in a subject with SpA comprising administering a MIF inhibitor to a subject with a higher likelihood of progression (e.g. those with elevated inflammatory parameters ESR/CRP, baseline existing NBF and smokers).

Accordingly, in another aspect the method is for treating an extra-articular symptom and/or condition associated with SpA.

In one embodiment, the extra-articular symptom and/or condition associated with SpA is an eye manifestation, optionally uveitis or iritis.

In another embodiment, the extra-articular symptom and/or condition associated with SpA is a skin manifestation, optionally psoriasis.

In another embodiment, the extra-articular symptom and/or condition associated with SpA is a gut manifestation such as IBD.

The MIF inhibitor can be any of the inhibitors described herein. The MIF inhibitor can be an inhibitor described in WO2010021693 and U.S. Pat. No. 9,643,922 (MIF Modulators), each of which are herein incorporated by reference.

In another embodiment, the MIF inhibitor is compound MIF098.

In one embodiment, the MIF inhibitor is a MIF098 analog, salt or derivative thereof.

In one embodiment, the MIF inhibitor is Ibudilast or an analog, salt or derivative thereof.

In another embodiment, the MIF inhibitor is anti-MIF antibody or binding fragment thereof that inhibits MIF activity by binding to its active site or by inhibiting its binding to the receptor CD74 and/or the complex CD74/CXCR2/CXCR4/CXCR7. For example, MIF098 prevents MIF-CD74 signaling by binding to the active enzymatic site of MIF that interferes with its interaction with CD74 through stearic hindrance. Ibudilast, binds adjacent to the active site and inhibits the tautomerase enzymatic activity. Other MIF inhibitors which interfere including anti-MIF antibodies or anti-CD74 antibodies, are also useful. For instance, Milatuzumab, a humanized monoclonal antibody (hLL1/IMMU-115) can be used for SpA. Derivatives of known anti-MIF or anti CD74 antibodies can also be used, for example, derivatives such as a single chain antibody, and/or modified form such as a fusion protein thereof having binding specificity for MIF or CD74 as the unmodified form.

In an embodiment, the MIF inhibitor is an anti-MIF antibody or antigen-binding portion thereof. In particular, the anti-MIF antibody can any antibody that inhibits interaction with CD74 or produces steric hindrance such that the function of MIF-CD74 complex is inhibited.

Treatment can for example improve pain, fatigue and/or disease progression.

Disease progression may be monitored by assessing any imaging changes including MRI and conventional X-rays, assessing sites or new sites of NBF, optionally neo-ossification in SIJ and/or axial joints, and/or ankylosis of the spine.

The MIF inhibitor can be suitably formulated into pharmaceutical compositions for administration to human subjects in a biologically compatible form suitable for administration in vivo.

The composition can comprise a pharmaceutically acceptable carrier, pharmaceutically acceptable diluent or pharmaceutically acceptable excipient.

In one embodiment, the dosage form is a solid dosage form. The MIF inhibitors described in WO2010021693 and U.S. Pat. No. 9,643,922 (MIF Modulators), and in particular MIF098 or MIF098 analogs, salts or derivatives thereof, can be formulated as solid dosage form, for example for oral administration

In one embodiment, the dosage form is a liquid dosage form. Anti-MIF or anti-CD74 antibody or binding fragments thereof, can for example be formulated for IV injection.

Suitable vehicles are described, for example, in Remington's Pharmaceutical Sciences (2003—20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

The inhibitors described herein can also be administered contemporaneously with a SpA therapy. For example, the SpA therapy can be a TNF inhibitor such as adalimumab, certolizumab, etanercept, golimumab or infliximab. The SpA therapy can optionally be an IL-17 inhibitor such as secukinumab or ixekizumab.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intraspinal, intracisternal, intraperitoneal, or oral administration.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

The MIF inhibitor can be for administration daily or twice daily.

Ibudilast for example, which has undergone clinical trials for asthma and multiple sclerosis has shown minimal toxicity. Similarly inhibition by MIF antibodies have also not resulted in major opportunistic infections or other limiting side effects. In an embodiment, the amount administered is an effective amount. For example, a single 30-mg dose followed by 14 days of 30 mg b.i.d was found to be generally safe in healthy adults (Rolan et al., Br J Clin Pharmacol 66,792-801(2008)) (ClinicalTrials.gov Identifier: NCT03489850). The dose may also be higher for example up to a 60 mg dose.

Also provided is a package comprising a MIF inhibitor or a CD74 inhibitor and a package insert. In one embodiment, the package insert indicates that the inhibitor, is indicated for the treatment of adults with early SpA. In another embodiment, the package insert indicated that inhibitor is indicated for the treatment of adults with moderate to severe active SpA, optionally adults who have had an inadequate response to conventional therapy.

As further expanded on in the Examples, a role for MIF in the initiation and progression of SpA through the modulation of type 3 immunity was demonstrated in a SpA mouse model. Substantial increases of MIF in blood and various tissues of curdlan-treated SKG mice were found. Furthermore, all observed SpA-like pathologies including spinal and peripheral arthritis, psoriasis-like dermatitis, blepharitis, ileitis, and NBF were successfully attenuated by targeting MIF, demonstrating that pharmacologic MIF blockade impacts most SpA disease manifestations. As further demonstrated, MIF inhibition is advantageous for example in axial SpA treatment over the current type 3 immunity cytokine blocking therapies, IL-23 inhibition being ineffective and IL-17A blockade showing limited efficacy (e.g., no benefit with colitis or iritis) (20)(21).

MIF is expressed upstream of multiple inflammatory cytokines (5), and without wishing to be found by theory, MIF inhibition may achieve more effective control of the cytokine-driven manifestations in different tissues. In addition, the data herein described shows that MIF possesses site-specific cytokine production regulatory activity. The data presented demonstrate that MIF is a key upstream molecule that site-specifically regulate cytokine production for example by modulating type 3 immune cells though direct and indirect mechanisms.

Interestingly, overexpression of MIF did not induce clear SpA pathologies in wild type C57BL/6 or BALB/c mice.

Overall, the data described herein demonstrates the importance of MIF in the induction and progression of SpA.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

Spondyloarthritis (SpA) is a chronic rheumatic disease characterized by severe inflammation in the spine, peripheral joints, intestine, skin and eyes. Although current treatment modalities including tumor-necrosis-factor (TNF) and interleukin (IL)-17 blockers could control inflammation, up to 40% of SpA patients don't adequately respond to any medications or lose their efficacies, resulting in severe pain, increased cardiovascular risk and deteriorating mental health. Macrophage migration inhibitory factor (MIF) is a pleiotropic cytokine that exhibits pro-inflammatory effects. MIF has functions in the regulation of immune responses and has been implicated in various inflammatory conditions. We recently discovered that serum levels of MIF were significantly elevated in Ankylosing Spondylitis (AS) patients compared to healthy controls. However, the specific role of MIF in SpA is largely unknown.

Methods: Curdlan (β-glucan) or MIF-plasmid (mini-circle) treated SKG mice (8-10 weeks) were used as SpA mouse models. The expression of MIF in serum or tissues was measured by ELISA, quantitative PCR (qPCR), western blotting, immunohistochemistry (IHC) and/or immunofluorescence (IF). MIF knockout (KO) SKG mice were generated as MIF deficiency mice. MIF inhibitor (MIF098) was used to block the function of MIF in SpA mouse models to assess the therapeutic or prophylactic effects in a curdlan-treated SpA mouse model. Populations of immunological cells were assessed by flow cytometry. Anti-Gr-1 monoclonal antibody (mAb) or isotype control mAb was used to block the function of neutrophils or monocytes. Clinical scores, histopathology and microCT imaging were used to assess the severity of inflammation in the various tissues of the mouse models.

Results: The expression of MIF and its receptor CD74 were significantly up-regulated in serum, spleen, ileum, sacroiliac and ankle joints of curdlan-treated SKG mice. MIF-overexpressed SKG mice injected with MIF-plasmid remarkably induced major SpA clinical features including colitis, psoriasis and arthritis in the axial and peripheral joints, while MIFKO SKG mice or blocking the function of MIF with MIF inhibitor (MIF098) dramatically suppressed or attenuated these manifestations, with decreased populations of Th17 and increased regulatory T (Treg) cells. We have also identified the cell populations (neutrophils) substantially producing MIF in the disease condition. Interestingly, adoptive transfer of these cells into non-disease control mice clearly exhibits SpA phenotype including arthritis, blepharitis and psoriasis. Furthermore, blocking the function of those cells with anti-Gr-1 antibody suppresses the SpA phenotype. Further details are provided in Example 2.

### Example 2

### Methods

#1. Animal model, treatments and clinical scoring: Female and male SKG mice (age 8-10 weeks) were injected with PBS, curdlan (3 mg/mouse, i.p.), MIF-plasmid (EEV, 5 μg/mouse, i.v., from systemic biosciences), or control-plasmid (5 μg/mouse, i.v., from systemic biosciences) to induce SpA phenotype and weekly observed the clinical manifestations of inflammation and NBFs over 8 weeks (n=10 mice/group). C57BL/6 and BALB/c mice (age 8-10 weeks) were also subjected to MIF- or control-plasmid injection to observe clinical symptoms. MIF- or control plasmid was administered by HDD tail vein injection as previously described (11). We also generated Mif−/− (KO) SKG mice by crossing SKG mice and BALB/c Mif KO mice (22).

After 1 or 4 weeks post-curdlan treatment, we injected MIF antagonist (MIF098; 40 mg/kg, twice/day, i.p.) (23) or control vehicle [PEG400 (Sigma, cat#91893) and HP-P-P-CD (Sigma, cat#C0926)] to assess the prophylactic and therapeutic effects of the MIF098 on the inflammation and NBFs in the SpA mouse model until 8 weeks post-curdlan treatment. Anti-mouse Gr-1 antibody (100 μg/mouse, Life Technologies) or isotype IgG monoclonal antibodies (100 μg/mouse, Life Technologies) were administered on day 3, 6, 9, 12 and 15 post-curdlan treatment.

Clinical scores for SpA-related manifestations were assessed based on severity scales (Table 1); arthritis, maximum 6 points; dermatitis, maximum 2 points; blepharitis, maximum 2 points). The scores were evaluated at least 2 independent scorers in a blinded fashion and final scores were the average of the observations. At the endpoint, ankle joints, spleen and lymph nodes (PLNs and MLNs) were dissected for FACS, one ankle was dissected prior to storage in RNA later for qPCR unless used for FACS, the other ankle was kept intact and fixed in 10% neutral buffered formalin for histopathological assessments. The upper tail spine, pelvis, eyes and ileum were dissected and fixed in 10% neutral buffered formalin for histopathology. For ankle digestion, skin was peeled off and toes (at distal phalanges) and tibia (˜0.5 cm above tibia) were cut off. After flushing bone marrow, the tissue was digested with RPMI culture media containing hyaluronidase and collagenase type VIII for 1 h at 37° C. in incubator. Following passing through 70 μm of cell strainer and RBC lysis treatment, cells were centrifuged and used for FACS analysis.

### #2. Histopathology

Ankle joints, tail spine, and pelvis were fixed in 10% neutral buffered formalin for at least 72 h, decalcified in 10% EDTA (BioShop) for 14-21 days and embedded in paraffin. Eyes and ileum were fixed in formalin for at least 72 h without decalcification. Serial sections (4 μm) were stained with hematoxylin and eosin (H&E; Fisher Scientific). To assess endochondral ossification, safranin O/ fast green staining was also applied to NBF in distal tibia as previously described (24, 25). For histological scores, multiple sections (three sections approximately 40-80 μm apart) per joint sample were evaluated by 2 independent scorers in a blinded fashion according to histological assessments as previously reported (11, 24). Final scores were the average of the observations, as previously reported55.

### #3. Immunohistochemistry (IHC)

IHC was performed as previously described (24, 25). Specifically, 4 μm sections were deparaffinized in xylene followed by a graded series of alcohol washes. Following proteinase K treatment (10 μg/ml) for 15 min, endogenous peroxide was blocked using 3% H₂O₂ for 30 min. Non-specific IgG binding was blocked by incubating sections with bovine serum albumin (BSA 1%) in PBS for 30 min. Sections were then incubated with primary antibodies, for MIF (abcam, cat# ab226166; Dilution 1:330), CD74 (abcam, cat# ab202844; Dilution 1:330), Sox9 (abcam, cat#185966; Dilution 1:330), type X collagen (abcam, cat# ab182563; Dilution 1:330), MMP13 (abcam, cat# ab39012; Dilution 1:330), Gr-1 (Invitrogen, cat#14-5931-82; Dilution 1:330) or rabbit IgG (Invitrogen, cat#02-610; Dilution 1:330) as an isotype negative control in a humidified chamber overnight at 4° C. temperature. After washing twice in water, the slides were incubated with their respective biotinylated secondary antibodies for 30 min. Signal was amplified with HRP conjugated secondary antibody followed by Vectastain Elite ABC kit (Vector Laboratories), as per the manufacturer's protocol, and counterstained with hematoxylin (Fisher Scientific).

### #4. Immunofluorescence (IF)

Similar to IHC, 4 μm sections were deparaffinized in xylene followed by a graded series of alcohol washes. Non-specific IgG binding was blocked by incubating sections with BSA 1% in PBS for 30 min. Sections were then incubated with primary antibodies, for MIF (abcam, cat# ab226166; Dilution 1:330), CD74 (abcam, cat# ab202844; Dilution 1:330) or rabbit IgG (Invitrogen, cat#02-610; Dilution 1:330) as an isotype negative control in a humidified chamber overnight at 4° C. temperature. After washing twice in water, the slides were incubated with secondary antibodies conjugated with either Texas Red (abcam, cat# ab6719) or Alexa fluor (abcam, cat# ab150113) for 30 min at room temperature. To test the expression of RORγt in ankle soft tissue, PE-conjugated primary antibody (BD, cat#562607) was used without secondary antibody. After washing, diluted DAPI solution was added to each well and incubated 2 minutes at room temperature. The slides were washed with PBS once and mounted with an anti-fade mounting media (DAKO). The slides were visualized using EVOS FL Imaging System (Life Technologies).

### #5. Curdlan or MIF Treatment for Cells and Tissues

In vitro splenocytes (1×10⁶/well) and ex vivo ankle soft tissue (0.5 g/well) were cultured in twelve-well plates with curdlan (1 μg/ml) or rmMIF (0, 10, 100 ng/ml) in RPMI or DMEM culture media containing 10% FBS and 1% Penicillin/Streptomycin at 37° C. in a humidified atmosphere of 5% CO₂ and 95% air for 0.5, 1, 1.5 or 24 h. RNAs or proteins were then extracted for qPCR and/or western blotting analysis, respectively.

### #6. Mouse naïve CD4+ and Treg Differentiation Assay into Th17

Fresh mouse naïve CD4+ T cells and Tregs (CD4+D25+) were isolated from spleen and PLNs of female SKG mice (8-10 weeks of age) using the mouse naïve CD4 (BioLegend, cat#480040) and Treg isolation kits (STEMCELL, cat#18783). The purity of CD4+CD25+T cells was 95.28%±0.11 (n=4, average±SEM). On day 0, a 96 well plate was coated with 50 μl of anti-mouse CD3ε (5 μg/ml, BioLegend, cat#100340) and incubated overnight in 4° C. After washing the plate with PBS on the next day, equal numbers of naïve CD4+ T cells or Tregs (2×10⁵/well) were cultured in the 96 well plate in the complete IMDM containing anti-mouse CD28 (5 μg/ml, BioLegend, cat#102116) alone or in combination with rmMIF (50 ng/ml, BioLegend, cat#599504) for 4 days. Equal numbers of naïve CD4+ T cells (4×10⁵/well) were also cultured with neutrophils (2×10⁵/well) isolated from curdlan-treated SKG mice for 4 days. The cells and culture supernatant were used for further analysis.

### #7. Mouse Treg Suppression Assay

Fresh antigen presenting cells (APCs) and CD4+CD25− T cells were isolated from spleen of female WT BALB/c mice (age: 8 weeks) using beads isolation kits (both are STEM CELLS; cat#18951 and cat#18783, respectively). The purity of CD4+CD25− T cells was 95.13%±0.19 (n=3, average±SEM). Fresh mouse Tregs (CD4+D25+) were isolated from either WT BALB/c, WT SKG, or Mif KO SKG mice (age: 8 weeks) as described above. Following the labelling with Cell Proliferation Dye eFluor450 (10 μM, eBioscience, cat#65-0842-85), equal amount of CD4+CD25− T cells (5×10⁴ cells) were co-cultured with irradiated APCs (2×10⁵ cells) and Tregs (10.0, 5.0, 2.5, or 1.25×10⁴ cells) in RPMI containing 10% FBS and 1.0 μg/ml of anti-CD3 for 72 h. Cell proliferation after stimulation for 72 h was assessed by flow cytometry.

### #8. In vitro Human Treg Differentiation Assay into Th17 and Cytokine Measurement

Human naïve CD4+ T cells and Tregs (CD4+CD25+CD127low) were isolated from PBMCs of healthy male controls without any history of back pain, arthritis, and joint injuries (age: 18-40, n=4 individuals in total) using the human naïve T cells isolation kit (STEM CELL, Cat #19555) and Treg isolation kit (STEM CELL, cat#18063), respectively. Isolated human Tregs (3×10⁴ cells/well) were cultured in the complete IMDM culture media containing ImmunoCult™ Human CD3/CD28/CD2 T Cell Activator (25 μl/ml, STEMCELL, cat#10970), and rhIL-2 (100 IU/ml, BioLegend, cat#589102) alone or in combination with or without rhMIF (50 ng/ml, BioLegend, cat#599404), rhIL-1β (25 ng/ml, BioLegend, cat#579402) and rhIL-23 (100 ng/ml, BioLegend, cat#574102) for 12 days. Each culture media was replaced ever 2-3 days. The cells were used for further analysis.

Released cytokine in cell culture supernatant on day 12 was quantified using the LEGENDplex Human Th17 Cytokine Panel (BioLegend, cat#741032) according to the manufacturer's instructions.

### #9. Cell Adoptive Transfer

Total Gr-1+ cells were isolated from bone marrow and spleen of curdlan-treated female SKG mouse at 8 weeks post curdlan using mouse CD11b+Gr1+ Isolation Kit (STEMCELL, catalog#19867). After the bead isolation, neutrophils (2×10⁶ cell) were isolated using FACS Aria III cell sorter (BD). Neutrophils (2×10⁶ cell) from female SKG mice treated with PBS were used as controls. After washing with PBS three time, the neutrophils or control neutrophilswere injected into Mif KO SKG mice at 1- and 2-week post-curdlan treatment through tail vein.

### #10. Flow Cytometry

For all panels, single cell suspensions were first stained with a fixable live dead stain (L/D NIR, Invitrogen, cat#L10119) as directed by the manufacturers. Cells were blocked with FcX (BioLegend, cat#101320) or Monocyte Blocker (BioLegend, cat#426102) prior to staining with surface antibodies. For experiments in which transcription factors were stained, cells were fixed and permeabilized with True-Nuclear kit (BioLegend, cat#424401) as directed. For experiments in which cytokines were stained, cells were fixed with a PFA buffer and permeabilized with intracellular staining buffer (BioLegend, cat#420801 and cat#421002) as indicated. Brefeldin A (BioLegend, cat#420601) with or without PMA/ionomycin (BioLegend, cat#423302) were used for in vitro stimulations to detect intracellular cytokines. Data were acquired on LSR II or Canto II (BD) and analyzed with FlowJo (version 10.6, Becton Dickinson).

### #11. Reverse Transcription and Quantitative Real-Time Quantitative PCR (qPCR)

RNA concentrations were determined using NanoVue (GE Healthcare Life Science). Following RNA quantification, equal amounts of RNA (1000 ng) were converted to cDNA using the QuantiTect Reverse Transcription PCR Kit (Qiagen) for mRNA, as per the manufacturer's protocol. For qPCR reactions, 5 ng of RNA per well was used for gene expression with primers and SYBR Green Master Mix (BIO-RAD) with primers and SYBR Green Master Mix Kit (Qiagen) according to the manufacturer's protocol. The reactions were incubated in 96 well plates (BIO-RAD) and performed in duplicate. Specificity of the amplified qPCR product was assessed by performing melting curve analysis on the LightCycler® 480 Instrument (Roche). The relative expression of PCR products was calculated by the 2-ΔCt method. All primers were designed using Primer3 online software. Data were normalized to GAPDH for mRNA analyses. The reference genes showed highly stable expression compared to other candidates for reference genes as previously reported55,56.

### #12. ELISA

The concentration of MIF in SKG mice or culture supernatant media and the concentration of IL-17A in the human Tregs culture media were assessed by mouse MIF ELISA kit (LEGEND MAX™ Mouse MIF ELISA Kit, BioLegend, cat# 44107) and Human IL-17A ELISA kit (LEGEND MAX™ Human IL-17A ELISA Kit, BioLegend, cat# 433917) were used, respectively. Samples were analyzed according to the manufacture's instruction.

### #13. Western Blot Analysis

Equal amount of cell lysates in RIPA buffer were applied to SDS-polyacrylamide gels (10%) for electrophoresis, as previously reported55,56. Separated protein was electroblotted onto polyvinylidene fluoride membranes. Membranes were blocked in 10 mM Tris-buffered saline (TBS) containing 5% skimmed milk and probed for 1.5 h with rabbit IgG primary antibodies (1:250) specific for MIF (abcam, cat# ab226166) and CD74 (abcam, cat# ab202844) or mouse monoclonal IgG for β-actin (1:1000; Sigma-Aldrich, catalog A1978) in blocking buffer. After washing the membranes with TBS containing 0.1% Tween-20 (TBS-T) 3 times, the membranes were incubated for 1 h at room temperature with HRP conjugated anti-rabbit (1:5,000; Sigma-Aldrich, catalog# SAB3700843) or anti-mouse (1:10,000; Sigma-Aldrich, cat#A2179) secondary antibodies in TBS containing 5% skimmed milk. Membranes were subsequently washed in TBS-T and protein bands were visualized with an enhanced chemiluminescence substrate (Clarify™ Western ECL Substrate, BIORAD and SuperSignal West Pico, Thermo Science) using a BIO-RAD Chemidocapparatus. Blots were scanned and signal intensity was quantified using Image J (National Institutes of Health, USA).

#14. Micro-CT: For assessments of bone formation and the temporal profile of bone structural changes, in vivo longitudinal micro-CT (SkyScan 1276, Bruker Corporation, Kontich, Belgium) were performed in curdlan-treated SKG mice, MIF PLM-injected SKG mice, MIF098-treated SKG mice or Mif−/− SKG mice accompanied by controls per group. At 8 weeks of curdlan or plasmid treatments, mice were euthanized with CO2 (1.3 L/min) in a cage and scans performed. All micro-CT scans were reconstructed with InstaRecon software (Champaign, Ill., USA) and screen captures taken of volume rendered CTvox (Bruker Corporation, Kontich, Belgium) images.

### #15. Statistical Analysis

All statistical analysis performed with GraphPad Prism8 (San Diego, Calif., USA). Data tested for normality before statistical test selected. Statistical analysis comparing two treatment groups with parametric and non-parametric data were performed by two-tailed Student's T tests and Mann-Whitney U tests (unpaired) or Wilcoxon signed-rank tests (paired), respectively. Statistical analysis comparing multiple treatment groups with parametric were performed by one- or two-way analysis of variance followed by Tukey's post-hoc test. For statistical analysis comparing multiple treatment groups with paired or unpaired non-parametric were performed by Kruskal Wallis test or Friedman test followed by Dunn's multiple comparisons test, respectively. A value of P<0.05 were considered statistically significant for all comparison tests.

### #16. MIF Secretion Assay in Mouse Immune Cells

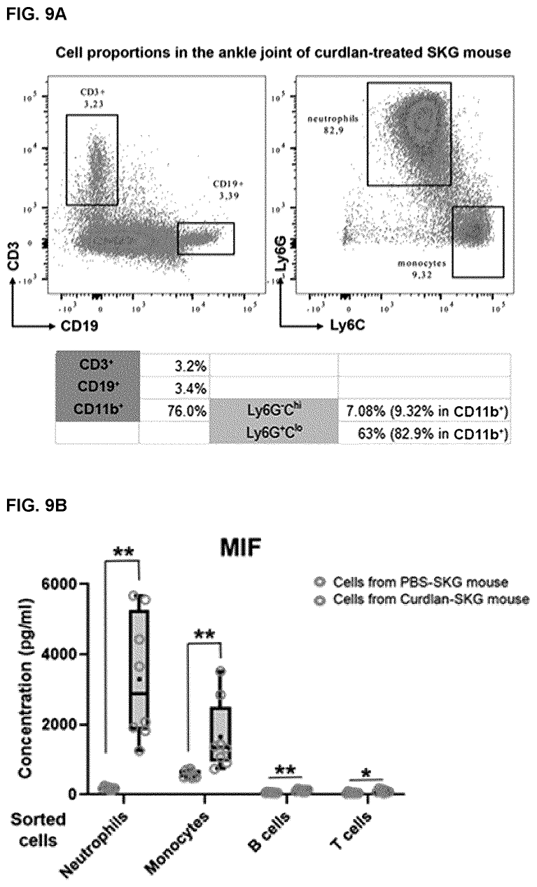

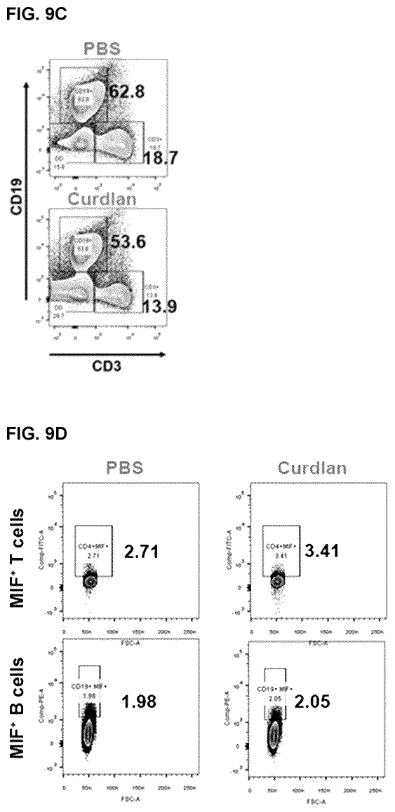

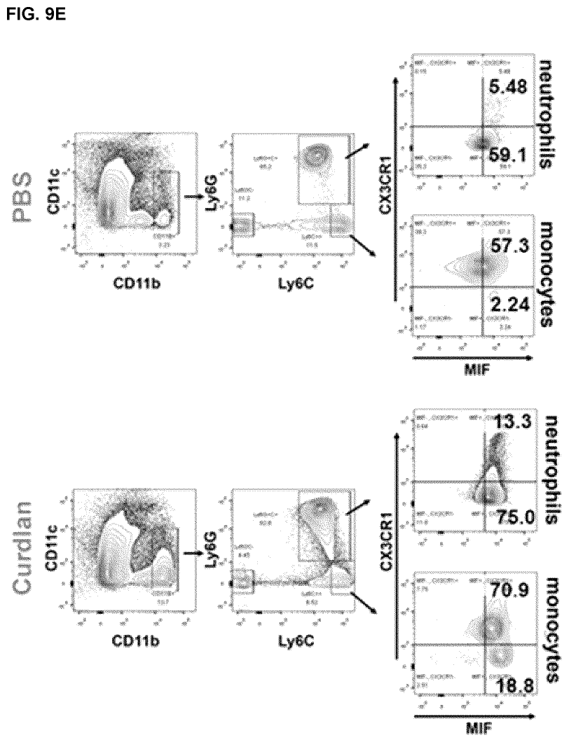

Mouse neutrophils, monocytes, B cells and T cells were isolated from bone marrow, spleen or PLNs of Mif+/+ or Mif−/− SKG mice and sorted by FACS. The number of each cell population (two million neutrophils, 0.22 million monocytes, 0.11 million B cells, and 0.11 million T cells per well) was determined based on the ration of inflamed ankle joint (FIGS. 9A-9E). The cells were immediately cultured in a 96 well plate containing Hank's Balanced Salt Solution (HBSS) with or without curdlan (1 μg/ml) in the presence or absence of anti-Dectin-1 mAb (100 ng/ml, InvivoGen, cat# mabg-mdect) or isotype control IgG2a mAb (100 ng/ml, Life Technologies, cat#16-4321-82) for 30 or 60 minutes. The level of MIF in the culture media was measured by enzyme-linked immunosorbent assay (ELISA) as described below and cell lysates were used for immunoblotting analysis.

### #17. MIF Secretion Assays in Human Neutrophils

Fresh human neutrophils were isolated from blood in SpA or healthy volunteers using EasySep Human Neutrophil Isolation Kit (STEMCELL Technologies, cat#17957). Cells were cultured in a 96 well plate (two million cells per well) containing HBSS for 60 minutes with or without lipopolysaccharide (LPS, 0.1 μg/ml for 60 minutes) or curdlan (1 μg/ml for 60 minutes). The level of secreted MIF into the culture media was measured by ELISA as described below.

### Results

### Curdlan-Treated SKG Mice Exhibit Inflammation and NBF with Increased Expression of MIF

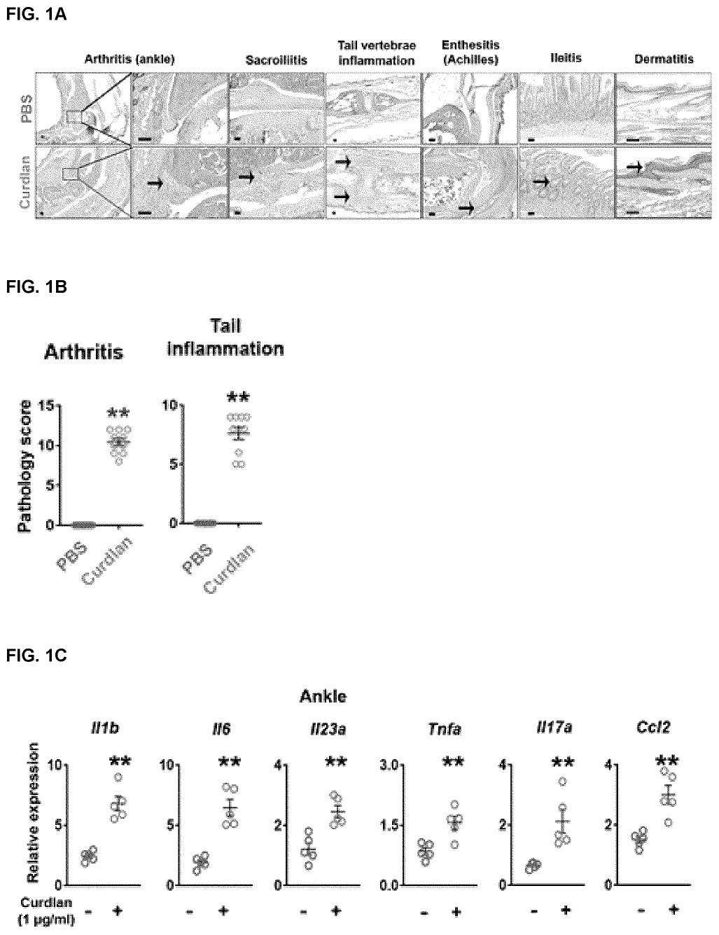

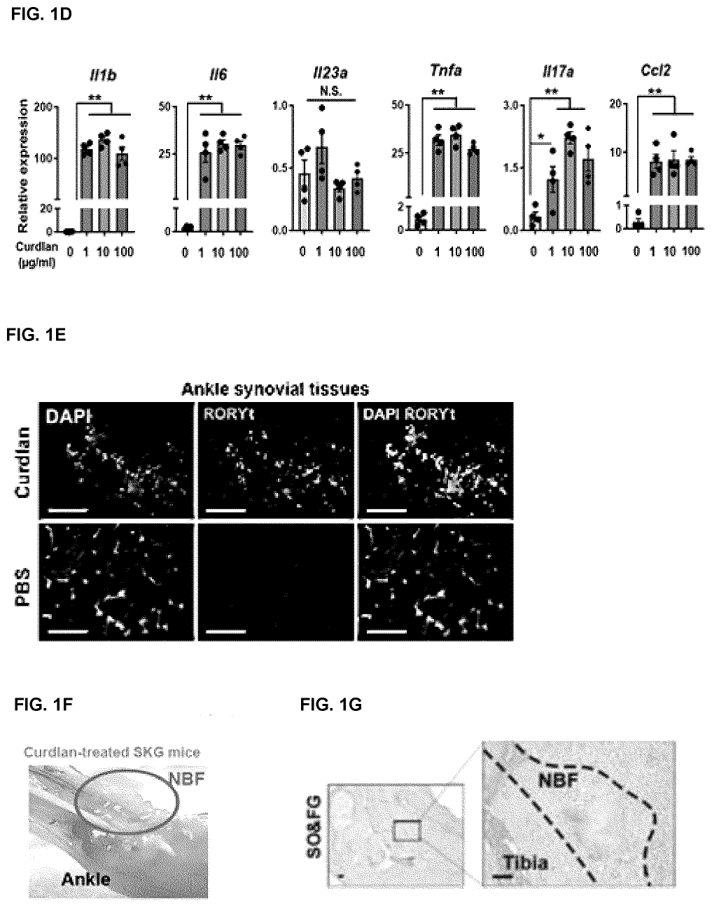

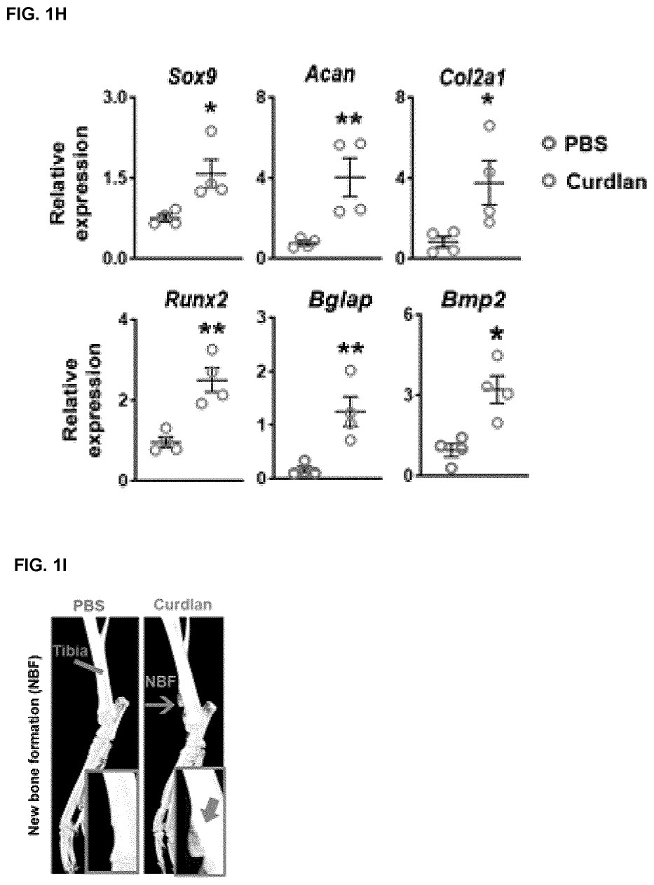

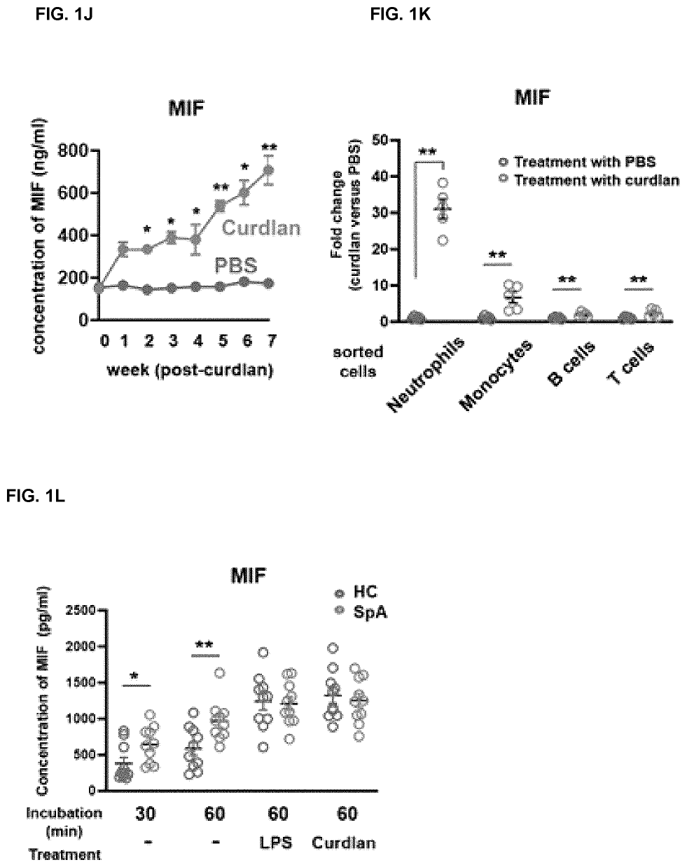

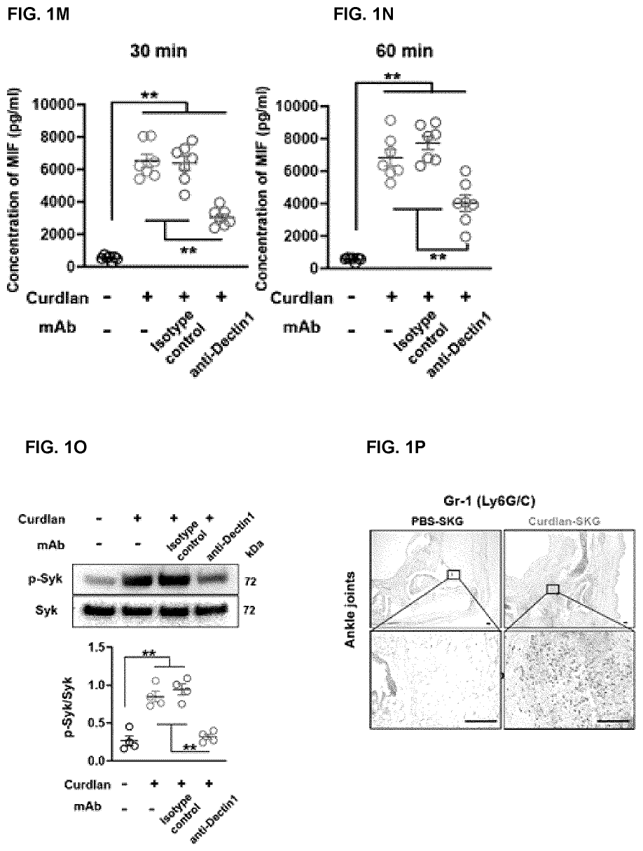

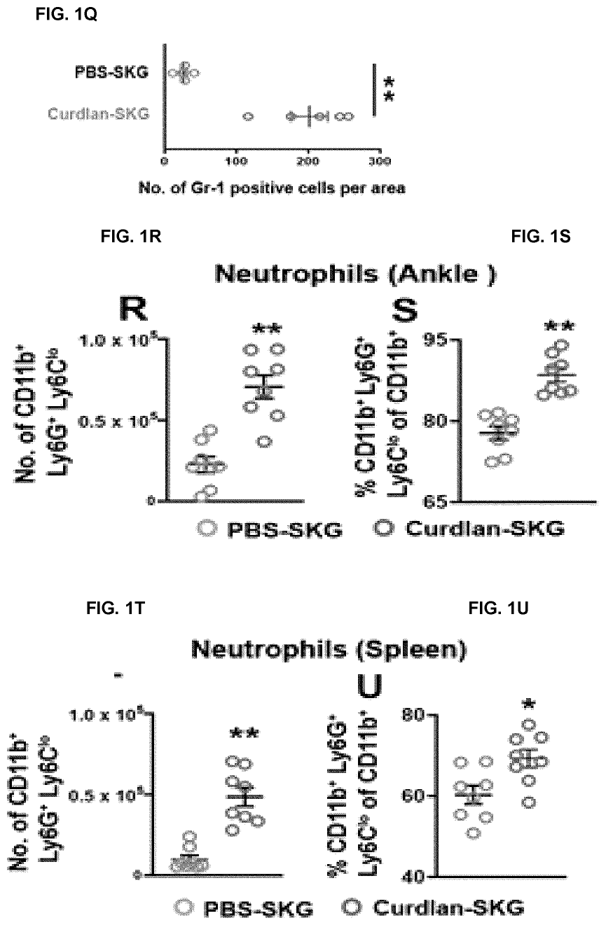

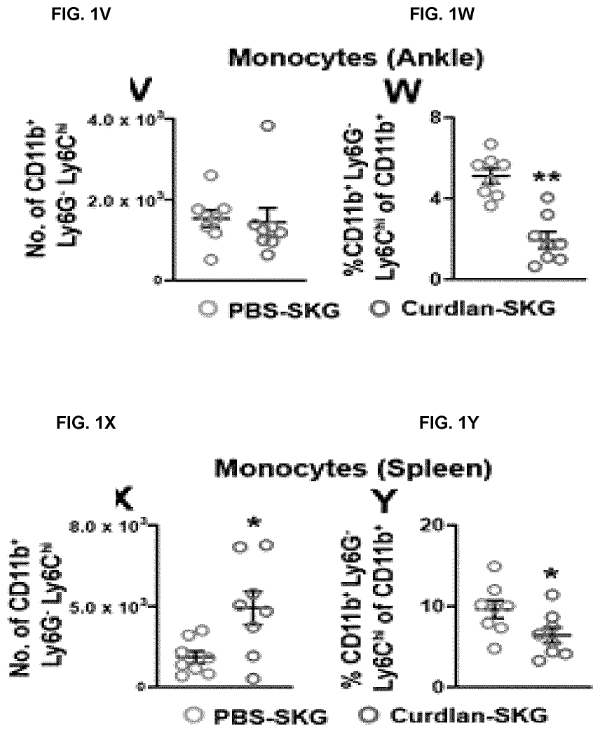

In line with previous reports (17), curdlan (β-glucan)-treated female SKG mice exhibited accelerated and more severe development of SpA-like clinical symptoms over 8 weeks, compared to male SKG mice; thus female SKG mice were primarily used for subsequent studies, unless indicated. Histological tissue sections showed evidence of severe inflammation of the ankle, sacroiliac joint, tail vertebrae, enthesis, ileum and skin of SKG mice at 8 weeks post-curdlan treatment, whereas there was no evidence of such clinically-relevant or histological features in saline (PBS)-treated SKG mice (FIGS. 1A-1B).

To investigate gene expression of inflammatory markers in response to curdlan in vitro, ankle soft tissues or splenocytes were isolated from healthy SKG mice and cultured with curdlan or PBS for 24 hours. An increase in gene expression of major SpA-related inflammatory markers (Il1b, Il6, Il23a, Tnfa, Il17a and Ccl2) was observed in both joint tissues and splenocytes cultured with curdlan compared to PBS treatment, with the exception of Il23a in splenocytes (FIGS. 1C-1D). Cells expressing the major IL-17 transcription factor, RORγt, were also observed in ankle synovial tissues of curdlan-treated SKG mice (FIG. 1E).

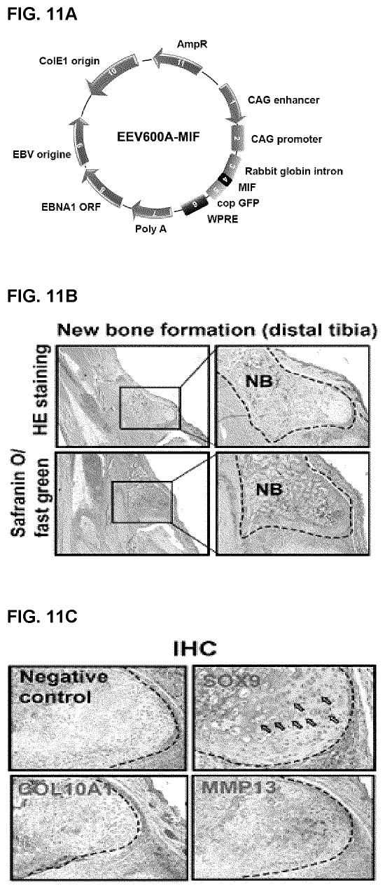

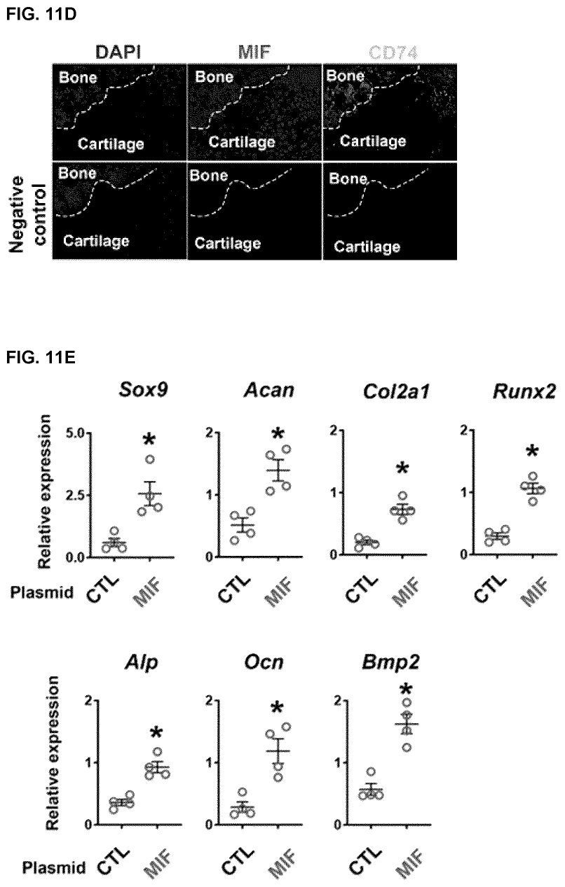

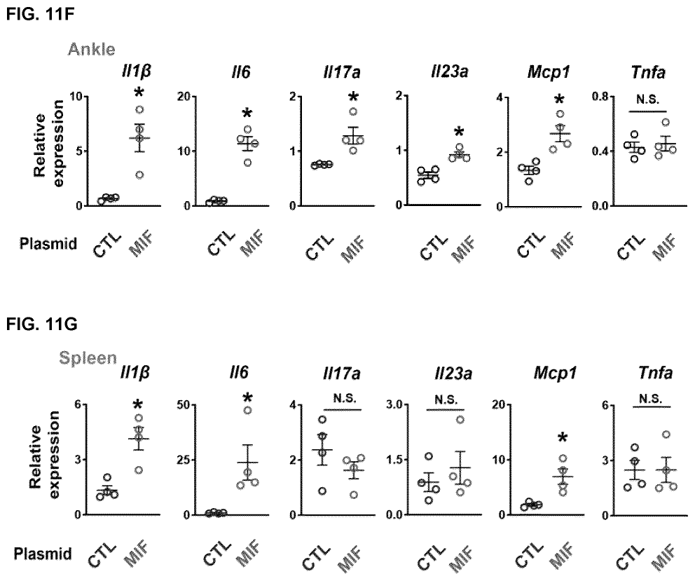

Abnormal NBF at entheseal sites (enthesophyte formation) following inflammation is also a cardinal feature of SpA. NBF was evident in the distal tibia of SKG mice at 8 weeks post-curdlan (FIG. 1F). It was determined that the NBF likely develops through the process of endochondral ossification (ECO) using histological and quantitative polymerase chain reaction (qPCR) analysis measuring ECO-related genes (FIGS. 1G-1H). Specifically, markers of chondrogenesis (Sox9), cartilage extracellular matrix (Acan and Col2a1), osteogenesis (Runx2), and bone formation (Bglap and Bmp2) were measured, all of which were increased in curdlan-treated SKG mice compared to control SKG mice. Established enthesophytes were also identified by micro-computed tomography (microCT) analysis (FIG. 11). Evident ankylosis was not consistently observed in the sacroiliac joints or tail; however, there was evidence of bone erosion and osteopenia in the sacroiliac and lumbar facet joints at 8 weeks post-curdlan treatment.

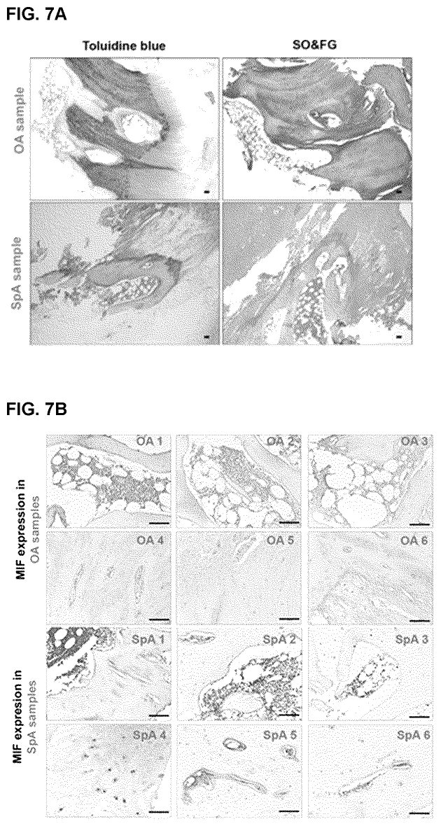

Consistent with SpA patients (6), the concentration of MIF in serum was increased in curdlan-treated SKG mice compared to PBS-treated SKG mice (FIG. 1J). Increased expression of MIF in human spinal tissues isolated from SpA patients compared to those from OA patients was also found (FIGS. 7A-7B). Together, these findings suggest that increased expression of MIF and CD74 may play important roles in the pathogenesis of SpA.

### MIF is Produced Predominantly by Neutrophils through the Curdlan-Dectin-1-p-Syk Axis

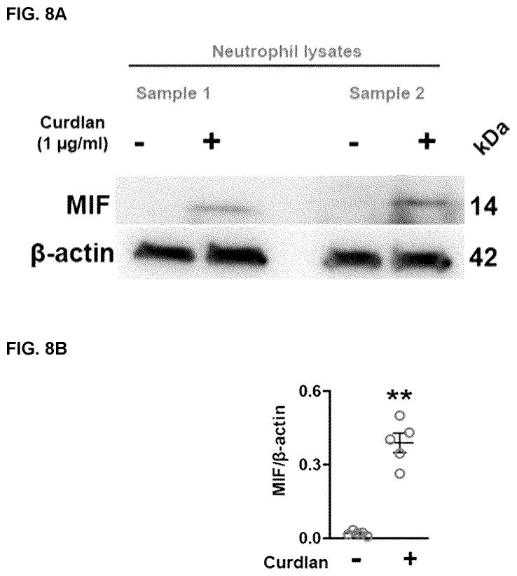

In vitro culture of the major immune cell lineages isolated from healthy SKG mice revealed that after 60 minutes of curdlan treatment, neutrophils (CD11b⁺Ly6G⁺Ly6C^{lo}) substantially increased secretion of MIF, whereas monocytes (CD11b⁺Ly6G⁻Ly6C^{hi}), CD19⁺ B cells, and CD3⁺ T cells showed milder increases (FIG. 1K). Despite the rapid release of MIF, immunoblotting analysis confirmed increased intracellular protein expression of MIF in neutrophil lysate (FIGS. 8A-8B), suggesting a rapid turnover from the production to release of MIF in SKG neutrophils upon activation by curdlan. Similar to the findings in SKG mice, human peripheral neutrophils freshly isolated from SpA patients secreted greater amount of MIF compared to those from healthy individuals, albeit no difference was observed after stimulation with lipopolysaccharide (LPS) or curdlan (FIG. 1L). These data suggest that neutrophils are one of the primary cell sources for the production of MIF in SpA.

The potential mechanism of how MIF is released from neutrophils in SKG mice was explored. It has been established that curdlan binds to the innate pattern recognition receptor Dectin-1 and promotes the expression of pro-inflammatory cytokines through phosphorylation of spleen tyrosine kinase (p-Syk), a downstream transducer of Dectin-1 (26-28). To determine the mechanism of MIF secretion in neutrophils, neutrophils were isolated from healthy SKG mice and treated the cells with or without curdlan in the presence or absence of anti-Dectin-1 neutralizing monoclonal antibody (anti-Dectin-1 mAb) or isotype control mAb in vitro. SKG neutrophils promptly secreted MIF into the culture media upon the stimulation with curdlan, whereas secretion was partially attenuated by anti-Dectin-1 mAb (FIGS. 1M-1N). Immunoblotting analysis also confirmed increased expression of p-Syk in response to curdlan, and the increase was reduced by anti-Dectin-1 mAb (FIG. 1O). These results demonstrate the underlying mechanism by which curdlan binding to Dectin-1 induces the release of MIF from neutrophils through increased phosphorylation of Syk.

### MIF-Producing Neutrophils Profoundly Expand in the Inflamed Tissues of SKG Mice

In addition to MIF secretion, confirming expansion of MIF-producing neutrophils in inflamed tissues is critical to substantiate neutrophils as key reposits of MIF that provoke inflammation in SKG mice. Gr-1⁺(Ly6G⁺/Ly6C⁺) cells, chiefly neutrophils and monocytes, were expanded in the ankle joints of curdlan-treated SKG mice compared to PBS-treated SKG mice (FIGS. 1P-1Q). Flow cytometry analysis further determined that the proportion and frequency of neutrophils were significantly increased in both ankle (P=0.0006, proportion; P<0.0001, frequency) and spleen (P=0.0002, proportion; P=0.0143, frequency) of curdlan-treated SKG mice compared to control SKG mice (FIGS. 1R-1U). In contrast, the frequency of monocytes was mildly, but significantly, decreased in ankle tissues (P=0.0011) or spleen (P=0.0409) of curdlan-treated SKG mice, however, with increased overall monocyte proportions in spleen (P=0.0281) (FIGS. 1V-1Y).

To confirm that neutrophils were the dominant cells producing MIF in inflamed tissues, cells from ankle joints of curdlan-treated SKG mice were isolated and sorted into neutrophils, monocytes, B cells, and T cells. Among live cells, more than 60% were neutrophils, followed by 7% monocytes, and 3 to 4% B and T cells each (FIG. 9A). Despite all cell populations increasing MIF release in response to curdlan treatment in SKG mice, considering MIF concentration from each cell population using population ratios (Neutrophils:Monocytes:B cells:T cells=9:1:0.5:0.5) further demonstrated that neutrophils were the dominant cell population that secreted MIF, followed by monocytes (FIG. 9B). In addition, neutrophils and monocytes were expanded and expressed MIF in popliteal lymph nodes (PLNs) of curdlan-treated SKG mice compared to control SKG mice (FIGS. 9C-9E). Together, these data firmly suggest that neutrophils are a primary cell population that profoundly expand and produce MIF in inflamed tissues of curdlan-treated SKG mice.

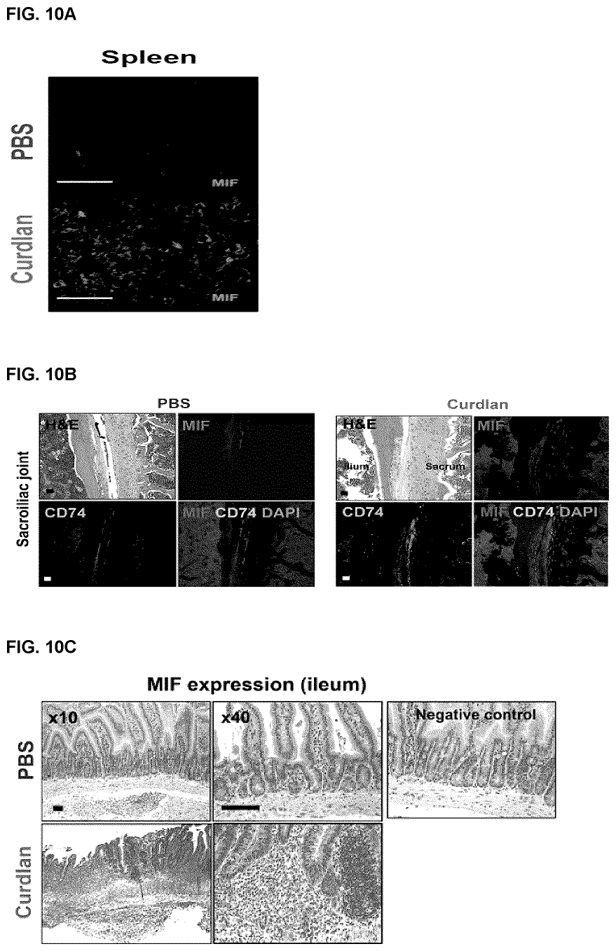

Next, whether MIF was increased in tissues of SKG mice was tested. Increased proportions of cells positive for MIF or CD74 were observed in spleen, sacroiliac joints, distal tibia with NBF, and ileum of curdlan-treated SKG mice compared to PBS-treated SKG mice, as determined by immunofluorescence (IF) and immunohistochemistry (IHC) (FIGS. 10A-10C).

### Overexpression of MIF in SKG Mice Using MIF Enhanced Episomal Vector (EEV) Plasmid Induces a SpA-Like Phenotype with a Bimodal Increase in Serum MIF

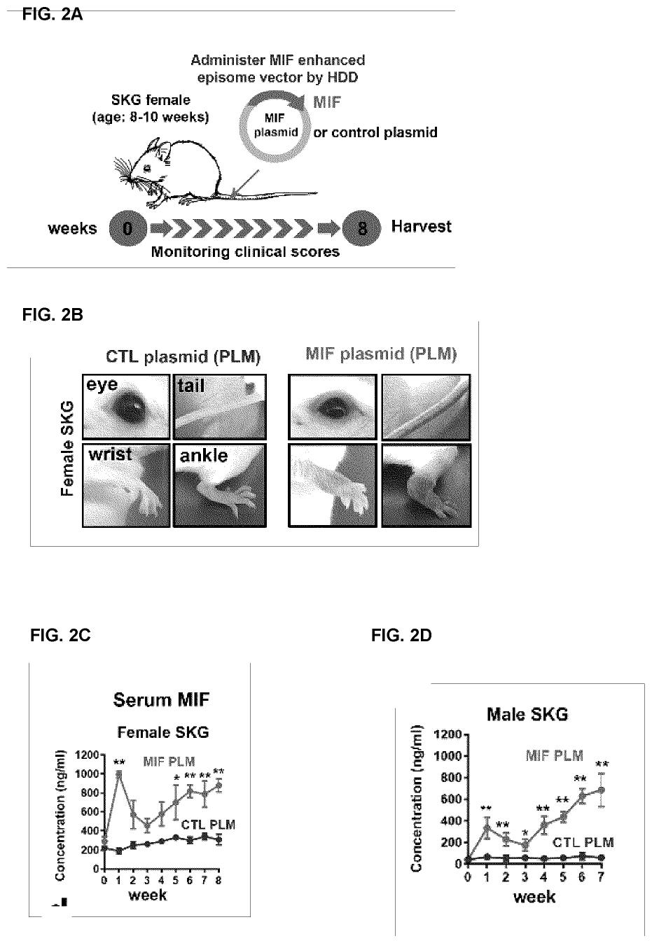

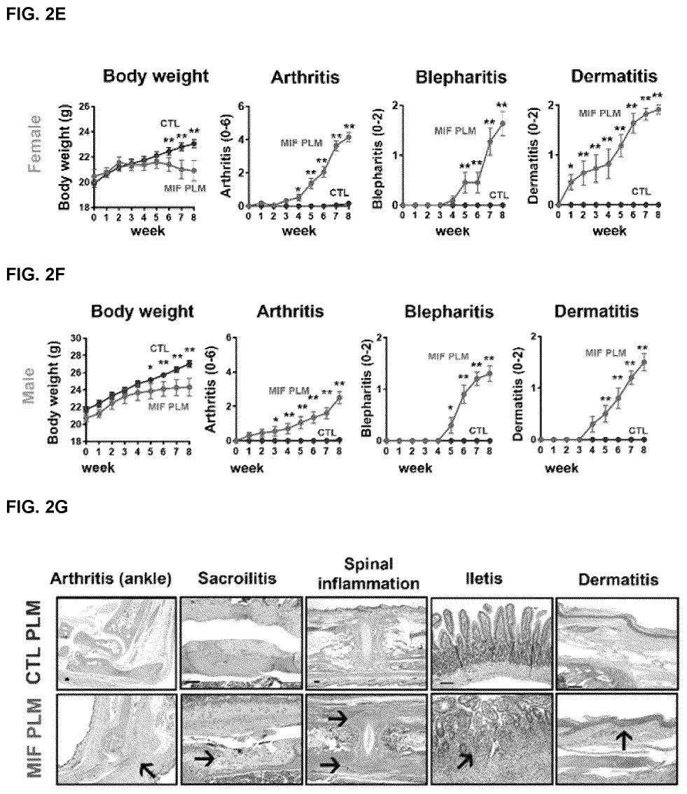

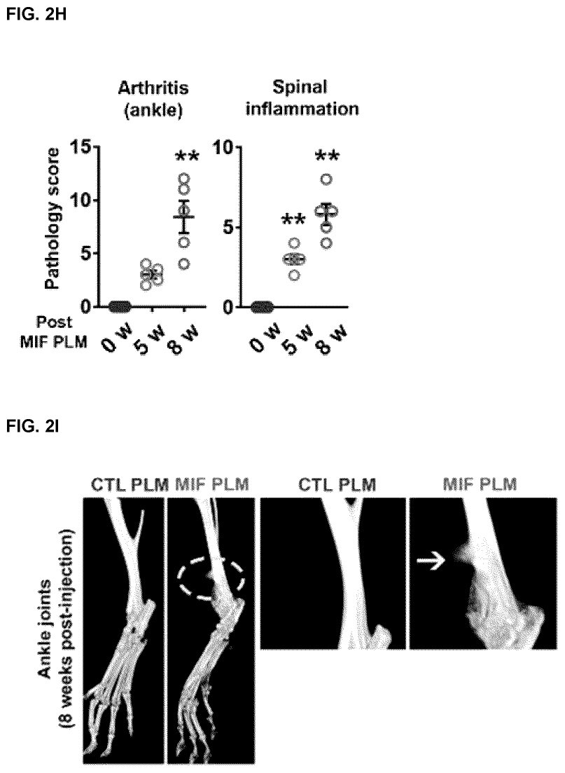

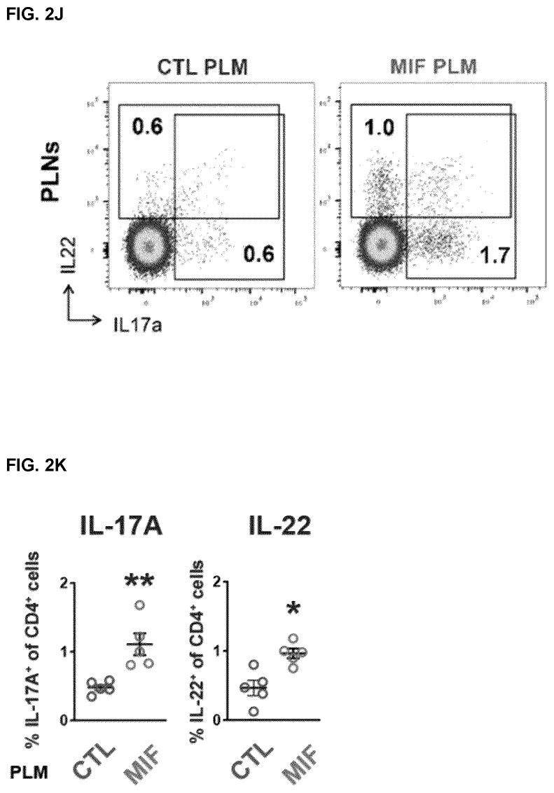

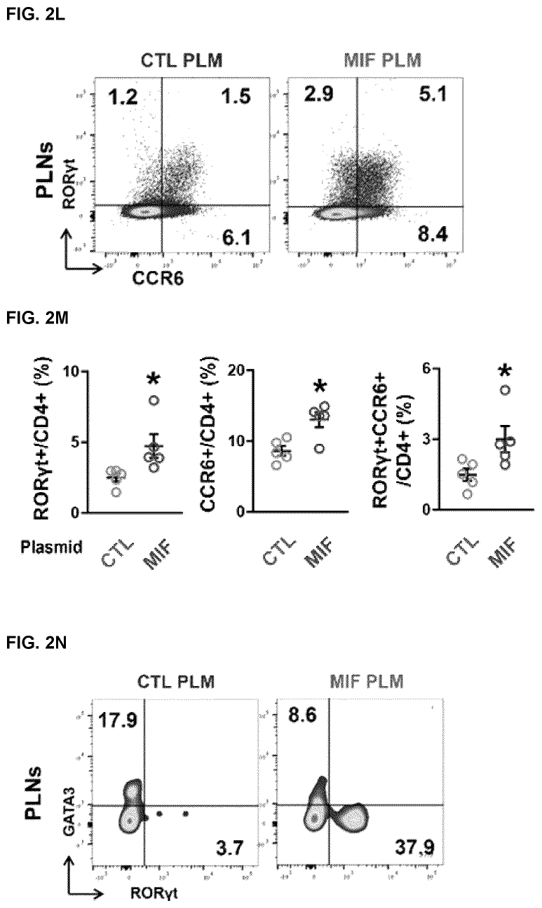

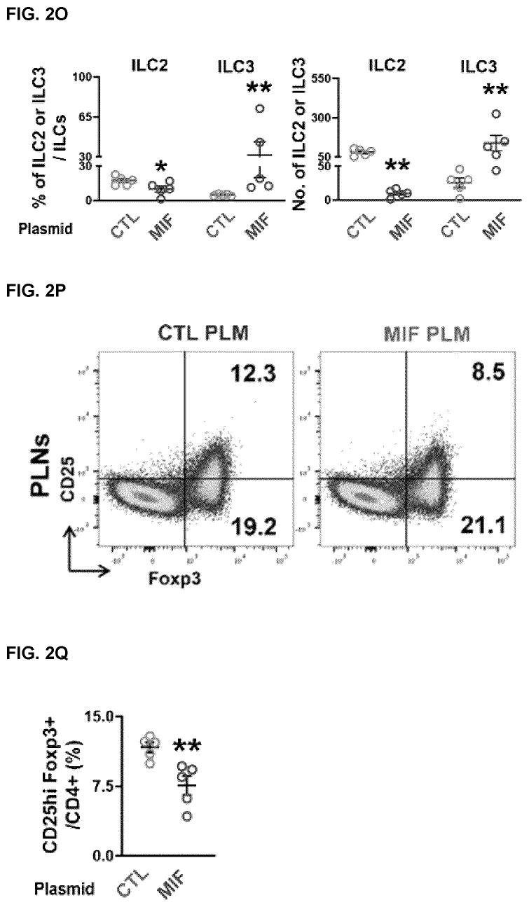

Although curdlan-treated SKG mice have SpA-like pathologies with increased expression of MIF, the potential contribution of MIF to these pathologies is unknown. Since curdlan-treated SKG mice also highly expressed other inflammatory markers (FIG. 1D) ], the effect of specific MIF overexpression instead of curdlan stimulation in SKG mice was assessed. Control-plasmid (CTL PLM) or MIF PLM (Enhanced Episomal Vectors: EEVs; FIG. 11) were injected into both SKG female and male mice through a hydrodynamic (HDD) tail vein injection (FIG. 2A). Following injection, clinical features were monitored for 8 weeks. SKG mice injected with MIF PLM had SpA-like pathologies including arthritis, psoriasis-like dermatitis and blepharitis with increased serum concentrations of MIF (FIGS. 2B-2F). Similar to curdlan-treated SKG mice, female SKG mice injected with MIF PLM had faster onset or severity of disease compared to male SKG mice injected with MIF PLM (FIGS. 2E-2F).

Increased inflammation in the ankle, sacroiliac joints, spine, ileum and skin were identified with histopathological scorings of ankle arthritis and tail spinal inflammation in MIF PLM-injected SKG mice compared to CTL PLM-injected mice (FIGS. 2G-2H). Moreover, using histological assessment, it was observed that the severity of clinical symptoms was lower in MIF PLM-injected SKG mice at 5 weeks compared to 8 weeks post-MIF PLM injection, evidenced by mild versus moderate-severe inflammation of the ankle joint and tail spine (FIG. 2H).

Since serum concentrations of MIF increased bimodally (initially peaking at 1 week and again increasing between 4-5 weeks post-MIF PLM injection in both female and male SKG mice; FIGS. 2C-2D), it was suspected that the second phase of increased MIF might be endogenous MIF expression originating from host cells, independent of MIF PLM. To test this, the concentration of MIF in the serum of female Mif−/− knockout (KO) SKG mice was monitored and it was confirmed that MIF PLM delivery increased serum MIF levels up to 4 weeks without development of evident clinical symptoms and did not induce a second phase of increased serum MIF. These findings suggest that MIF PLM delivered to SKG mice induces SpA-like pathologies by promoting host-derived MIF expression.

As MIF-overexpressing SKG mice exhibited SpA-like features, whether MIF-overexpressing BALB/c or C57BL/6 mice injected with MIF PLM develop SpA-like pathologies was also tested. No clear evidence of SpA-like characteristics including arthritis and spinal inflammation was observed, indicating that SKG mice are uniquely predisposed to development of MIF-induced SpA-like pathologies.

### Overexpression of MIF in SKG Mice Induces NBF in the Distal Tibia through the Process of ECO

It was previously shown that serum MIF levels were significantly elevated in AS patients with rapid ankylosis progression compared to slow progressive AS or healthy controls (6); however, the specific role of MIF on NBF in SpA is unclear. In MIF PLM-injected SKG mice, NBF in the distal tibia at 8 weeks post-injection was clearly observed, as assessed by microCT imaging (FIG. 2I).

Similar to curdlan-treated SKG mice, NBF in MIF PLM-injected SKG mice likely developed through the process of ECO (FIG. 11B), evidenced by increased expression of markers of chondrogenesis (Sox9), chondrocyte hypertrophy (Col10A1 and Mmp13), cartilage extracellular matrices (Acan and Col2a1), osteogenesis (Runx2), and bone formation (Alp, Ocn, and Bmp2), accompanied by highly expressed MIF and CD74, as assessed by IHC, IF and/or qPCR (FIGS. 11C-11E). These results demonstrate that MIF PLM delivery to SKG mice is adequate to induce NBF, likely through the process of ECO.

### MIF-Overexpressing SKG Mice Have Modified Inflammatory Cytokine Expression and Inflammatory Cell Populations

The expression of SpA-related inflammatory markers including Il1β, Il6, Il23a, Tnfa, Il17a and Mcp1 in the ankle soft tissues or spleen isolated from either MIF PLM- or CTL PLM-injected female SKG mice was evaluated. In the ankle soft tissues, a significant increase in the expression of Il1β, Il6, Il23a, Il17a and Mcp1, but not Tnfa was observed (FIG. 11F). Significantly increased expression of Il1β, Il6, and Mcp1 was also observed, but not Il23a, Il17a, and Tnfa in the spleen (FIG. 11G).

To further assess the expression of pro-inflammatory cytokines induced by MIF, CD4+ T cells from popliteal lymph nodes (PLNs), mesenteric lymph nodes (MLNs) and spleen in SKG mice were isolated and treated with either MIF PLM or CTL PLM. In line with the gene expression analysis, CD4+ cells with intracellular expression of IL-17A and IL-22 were significantly increased in PLNs of SKG mice injected with MIF PLM compared to SKG mice injected with CTL PLM (FIGS. 2J and 2K).

After determining that MIF PLM-treated SKG mice had enhanced expression of select inflammatory markers, SpA-related immune cells in the PLNs and spleen were also evaluated. The percentage of Th17 lineage cells (CCR6+ and/or RORγt+ in CD4+ cells) was significantly increased in PLNs of MIF PLM-treated SKG mice compared to CTL PLM-treated SKG mice (FIGS. 2L and 2M). Interestingly, ILC3s (CD3−Lin−CD90.2+RORγt+), a cell population known to produce IL-17 and IL-22 (12), were also increased in PLNs of MIF PLM-injected SKG mice (FIGS. 2N and 2O) while the total number and percentage of ILC2s (CD3−Lin−CD90.2+GATA3+) were significantly decreased in PLNs of MIF PLM injected SKG mice. Since ILCs are generally considered tissue-resident cells (29), ILCs in ankle soft tissue of MIF PLM-injected SKG mice were also assessed. It was found that all ILC1, ILC2s and ILC3s were significantly decreased in MIF PLM-injected SKG mice compared to CTL PLM-injected SKG mice.

The percentage of Tregs (CD4+CD25hiFoxp3+) was reduced in PLNs of MIF PLM-injected SKG mice compared to CTL PLM-injected SKG mice (FIGS. 2P and 2Q). Although there were no significant differences in the percentage of Th17 lineage cells in spleen, a significant decrease in the percentage of Tregs was observed. These results suggest that MIF modulates site-specific type 3 immune cells and Tregs during the development of SpA-like pathologies in SKG mice.

Overexpression of MIF Increases Inflammatory Macrophages and Decreases Patrolling Macrophages in Ankle Tissues

Since macrophages are reported to be important in the development of SpA-like pathologies in SKG mice (30), and their plasticity between inflammatory and patrolling characteristics are indispensable during the development of arthritis (31), changes in the proportions of inflammatory and patrolling macrophages in SKG mice 8 weeks post-CTL PLM or MIF PLM injection were evaluated. It was found that the frequency of inflammatory macrophages (CD11b+CD11c−Ly6ChiCX3CR1loCCR2+) in ankle soft tissues was significantly higher in MIF PLM-injected SKG compared to CTL PLM-injected SKG. In contrast, the frequency of patrolling macrophages (CD11b+CD11c−Ly6CloCX3CR1hiCCR2−) was significantly decreased in MIF PLM-injected SKG compared to CTL PLM. These results indicate that MIF stimulation regulates macrophage populations by enhancing inflammatory macrophages and decreasing patrolling macrophages in the inflamed joint tissues, which may also play a pivotal role in the pathogenesis of arthritis of SKG mice, in addition to type 3 immune response.

Mif KO Suppresses the Severity of SpA-Like Pathologies Induced by Curdlan in SKG Mice

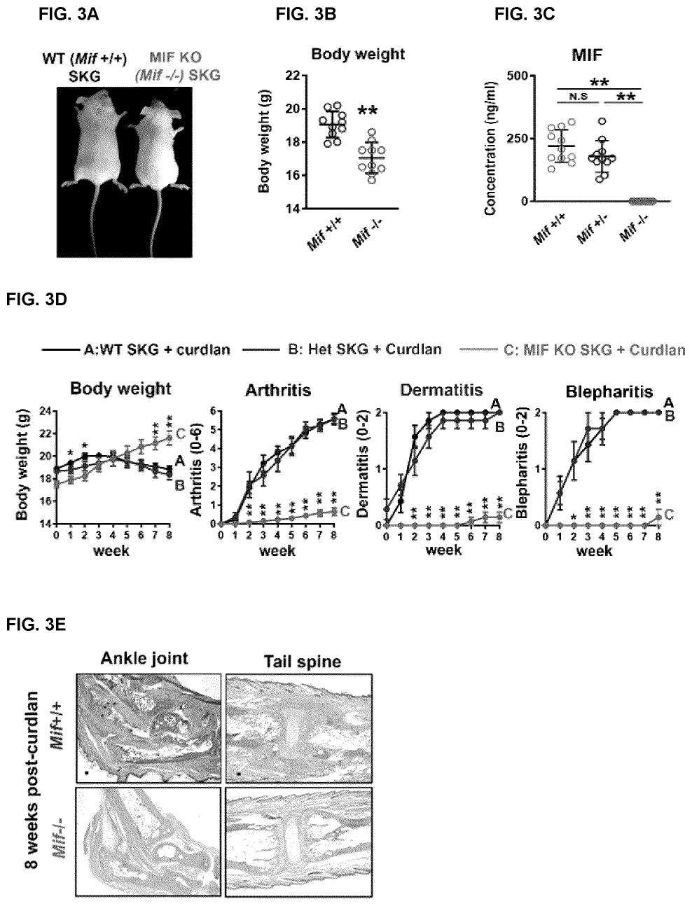

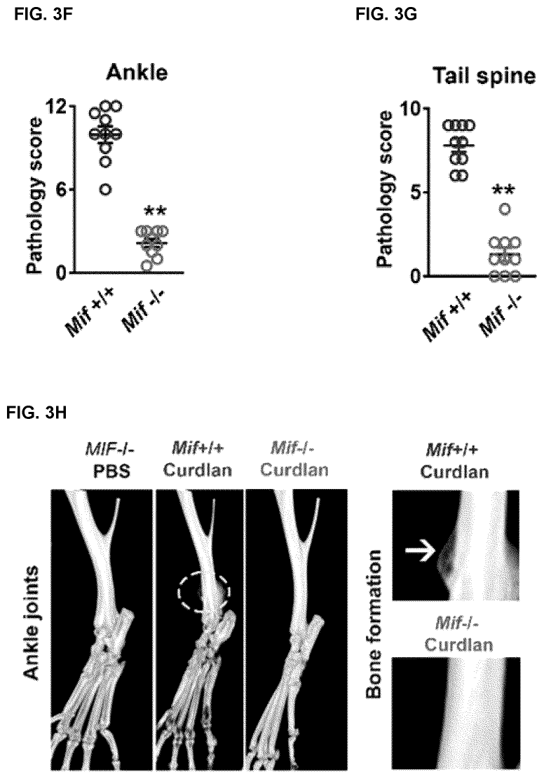

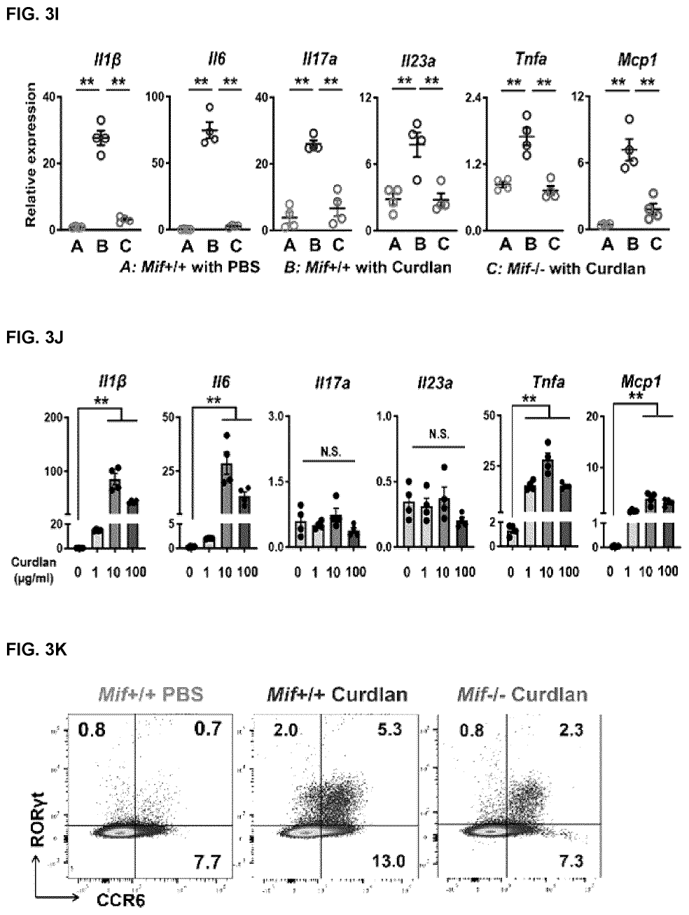

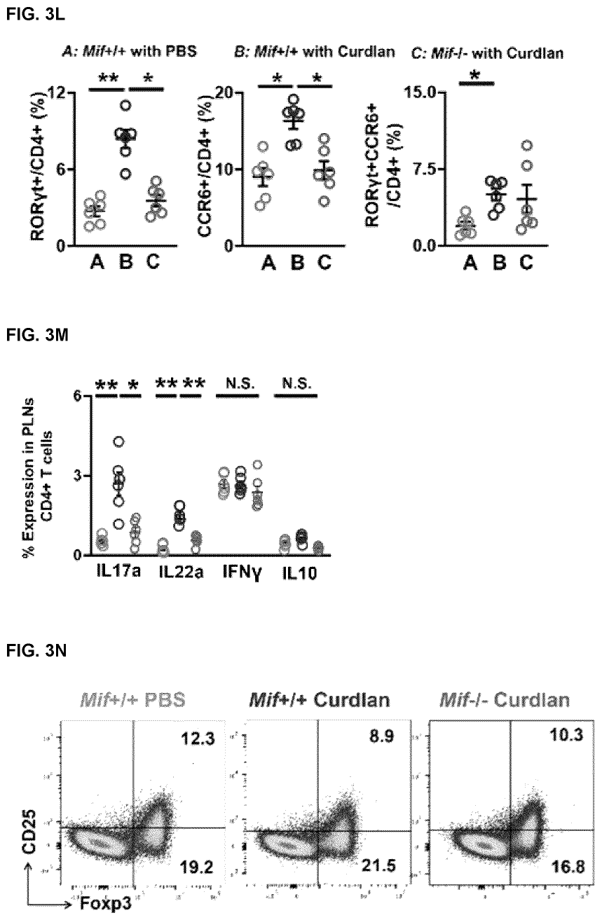

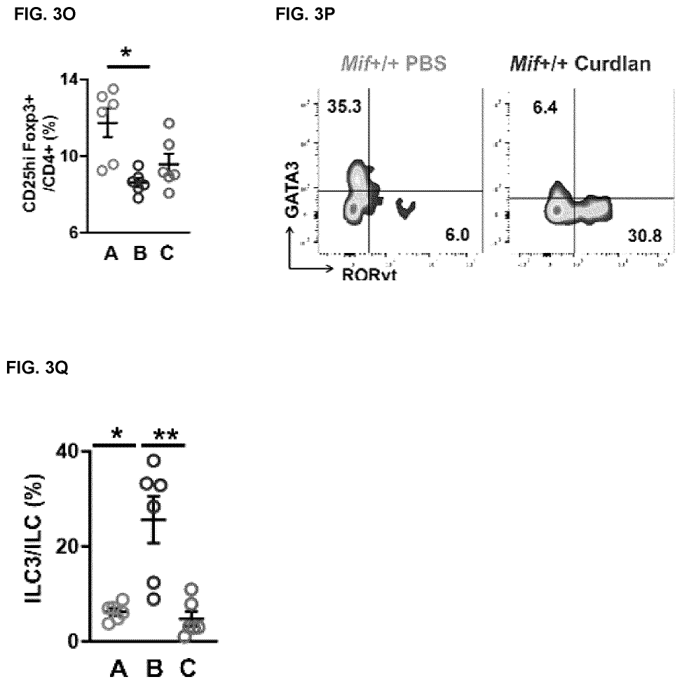

Since MIF-overexpressing SKG mice caused SpA-like pathologies, whether Mif KO (Mif−/−) SKG mice demonstrated a decreased severity of SpA phenotype compared to wild type (WT; Mif+/+) SKG mice following curdlan treatment was evaluated. Mif KO SKG mice were generated by crossing Mif−/− BALB/c mice with Mif+/+ SKG mice. Mif KO SKG mice showed approximately 10% lower body weight compared to WT SKG (FIGS. 3A-3B). It was confirmed that Mif KO SKG mice had no protein expression of MIF, as assessed by ELISA (FIG. 3C). Furthermore, no significant difference was found in serum concentrations of MIF between WT and heterozygous (Het; Mif+/−) SKG mice (FIG. 3C).

SpA-like clinical features assessed by clinical scoring were monitored for 8 weeks. Compared to WT or Het SKG mice, Mif KO SKG mice exhibited substantially lower scores for arthritis, dermatitis and blepharitis following curdlan treatment (FIG. 3D. It was also confirmed that curdlan-treated Mif KO SKG mice showed milder arthritis (ankle) and spinal inflammation compared to curdlan-treated WT SKG mice, as assessed by histopathology (FIGS. 3E-3G). Based on microCT imaging, NBF was observed in the distal tibia of curdlan-treated WT SKG mice but was absent in curdlan-treated Mif KO SKG mice at 8 weeks post-treatment (FIG. 3H).

### Attenuation of Curdlan-Induced Inflammation in Mif KO SKG Mice

Gene expression of SpA-related inflammatory markers in ankle joints of WT SKG and Mif KO SKG mice at 8 weeks post-curdlan treatment was evaluated. Consistent with previous results, curdlan treatment significantly increased the expression of Il1β, Il6, IL17a, IL23a, Tnfa and Mcp1 in ankle soft tissues of WT SKG mice. In contrast, curdlan-induced expression of these inflammatory cytokines was attenuated in ankle tissues of Mif KO SKG mice (FIG. 31). To determine expression of SpA-related inflammatory markers in response to curdlan in vitro, splenocytes from female Mif KO SKG mice were cultured and treated with curdlan for 24 h. Similar to WT SKG splenocytes, the expression of Il1β, Il6, Tnfa and Mcp1 were significantly increased in response to curdlan (FIG. 3J). Furthermore, unlike splenocytes from WT SKG mice, the expression of Il17a was not increased in splenocytes isolated from Mif KO SKG mice (FIG. 3J).

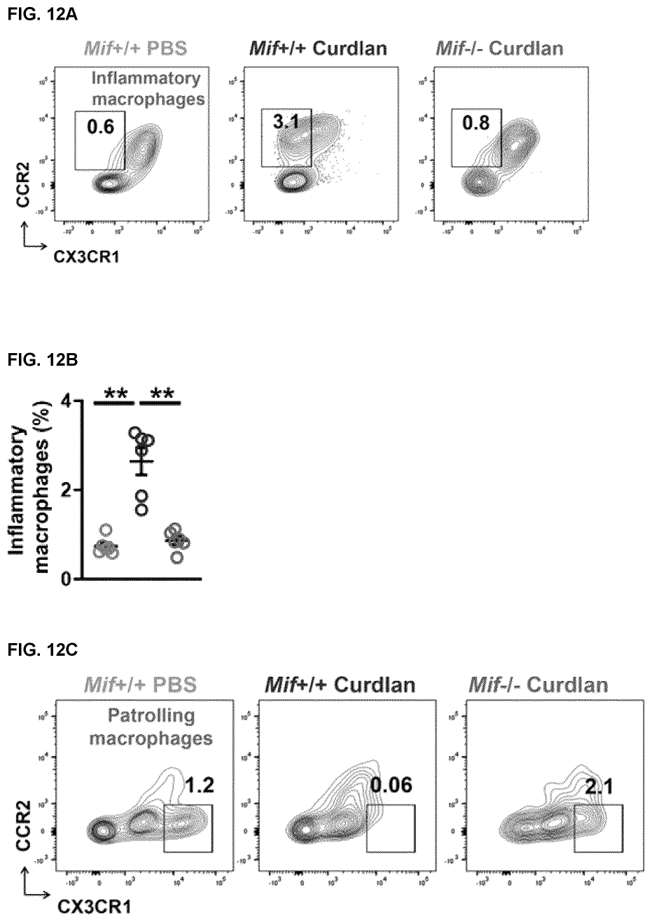

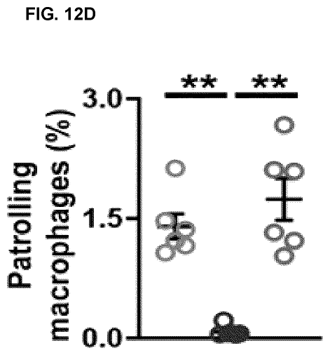

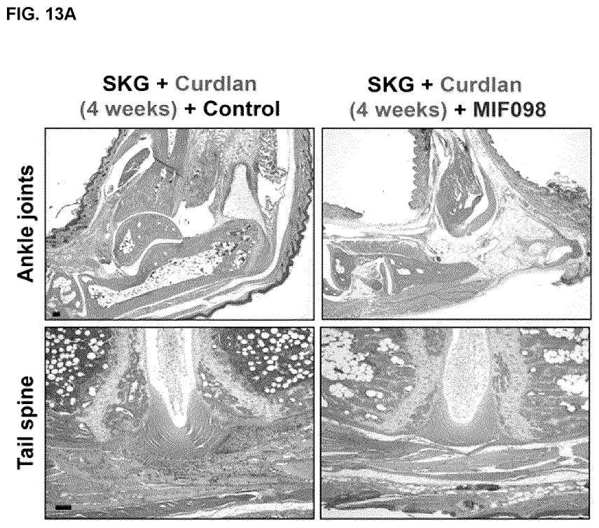

With respect to SpA-related immune cells in PLNs, the curdlan-induced increase in the frequency of Th17 lineage cells in WT SKG mice was attenuated in Mif KO SKG mice, with reduced populations of IL17A and IL22 positive CD4+ cells in PLNs of Mif KO SKG mice compared to WT SKG mice post-curdlan treatment (FIGS. 3K-3M). While curdlan increased the frequency of ILC3s and decreased the frequency of Tregs in PLNs of WT SKG mice, these population changes were also attenuated in Mif KO SKG mice (FIGS. 3N-3Q). Similarly, the curdlan-induced increases in inflammatory macrophages and reductions in patrolling macrophages in PLNs of WT SKG mice were attenuated in Mif KO SKG mice (FIGS. 12A-12D). Taken together, these results suggest that MIF regulates various SpA-related inflammatory markers and modulates type 3 immunity and macrophage phenotypes site-specifically, contributing to SpA

### Inhibition of MIF with a Pharmacological Antagonist (MIF098) Prevents or Attenuates SpA-Like Pathologies after Curdlan Treatment

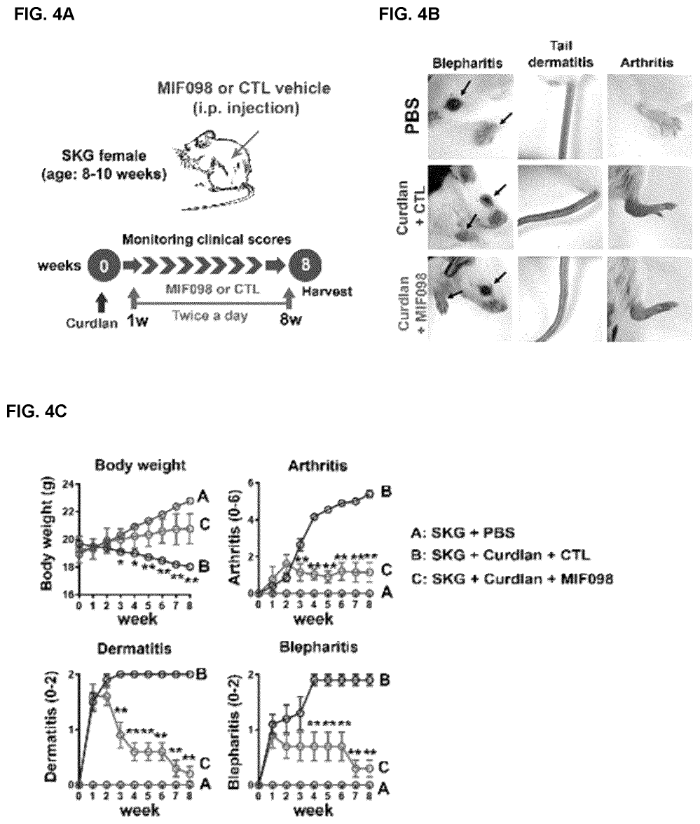

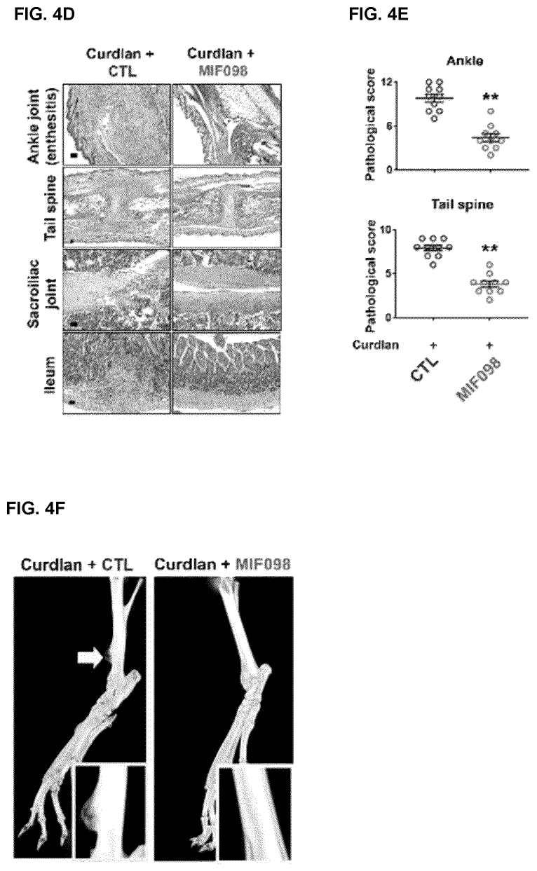

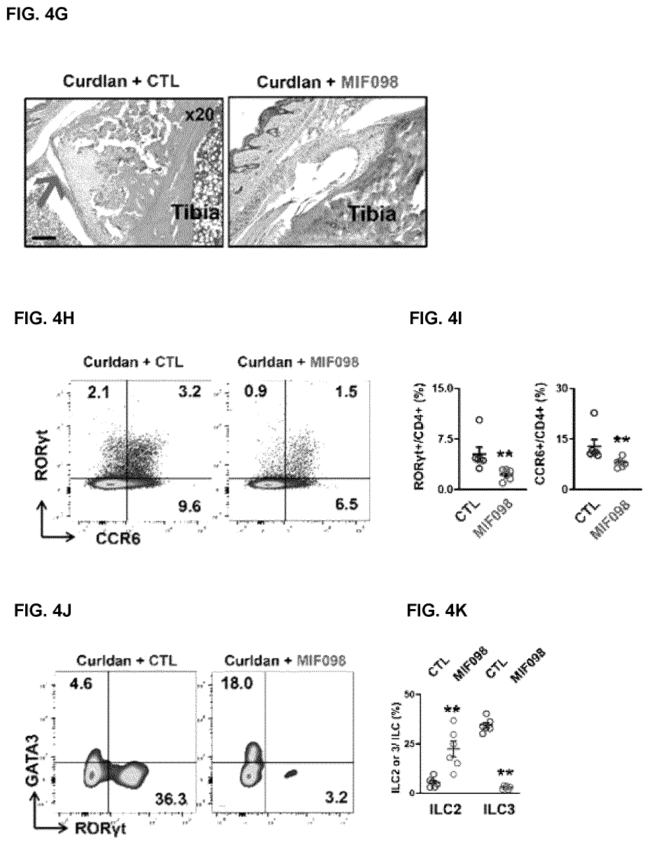

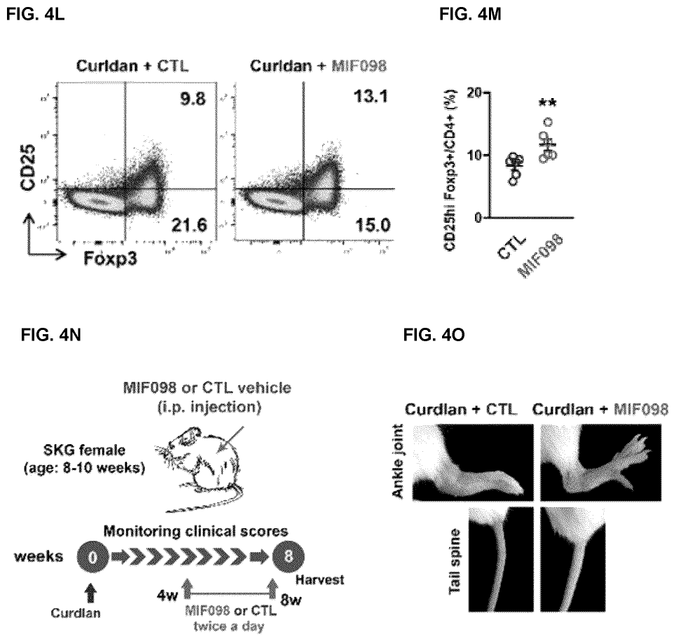

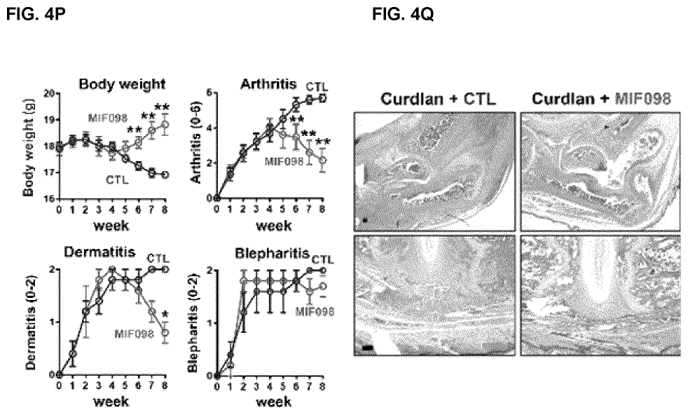

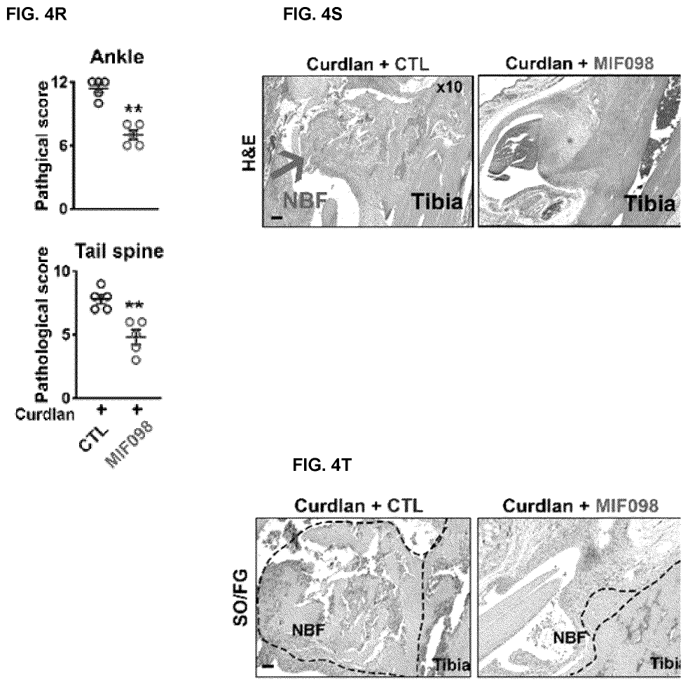

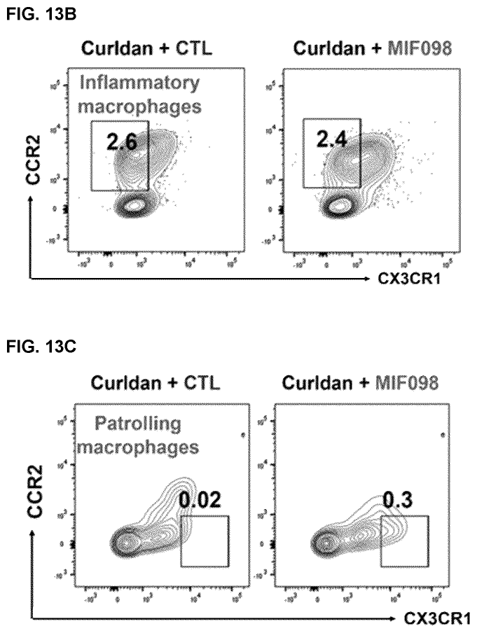

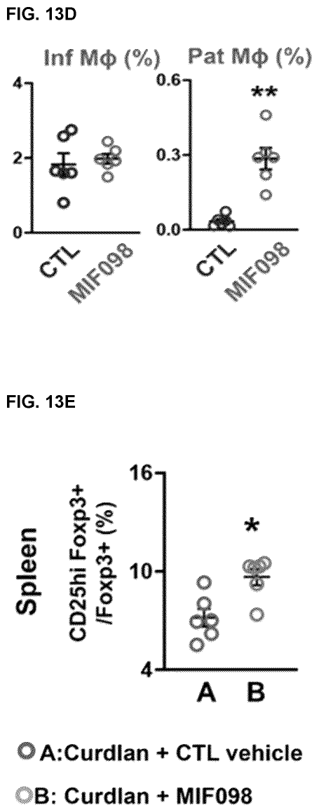

To assess the impact of pharmacologic MIF antagonism on SpA-like disease, a pre-clinical small molecule MIF antagonist (MIF098), which blocks the MIF/CD74 interaction (23), was injected into curdlan-treated female SKG mice. First, to test the prophylactic effect of MIF098, twice daily injections were started beginning one week post-curdlan treatment for 7 weeks (FIG. 4A). SKG mice injected with MIF098 showed reduced severity of arthritis, psoriasis-like dermatitis and blepharitis when compared to control SKG mice injected with vehicle control (CTL; FIGS. 4B-4C). Lower severity of inflammation in the ankle, tail spine, sacroiliac joints, and ileum was also observed with histology and histopathology scoring of ankle arthritis and spinal inflammation (FIGS. 4D-4E). Similar prophylactic effects of MIF098 on inflammation in the ankle joint and tail spine were observed even at 4 weeks post-curdlan. (FIG. 13A). Furthermore, microCT imaging and histological assessments showed that curdlan-treated SKG mice injected with MIF098 did not display an evident NBF, unlike curdlan-treated SKG mice injected with CTL that had clear NBF (FIGS. 4F-4G). These data suggest that MIF098 can prevent inflammation and NBF in curdlan-treated SKG mice.

Similar to Mif KO SKG mice, the frequency of Th17 lineage cells and ILC3s in PLNs were significantly deceased in curdlan-treated SKG mice injected with MIF098 compared to CTL (FIGS. 4H-4K), while ILC2s were remarkably increased. In addition, there was a significant increase in the percentage of Tregs in curdlan-treated SKG mice injected with MIF098 compared to CTL (FIGS. 4L-4M). Moreover, there was an increase in the percentage of patrolling macrophages in curdlan-treated SKG mice injected with MIF098 compared to CTL while no difference between groups was observed in the percentage of inflammatory macrophages in the ankle joints (FIGS. 13B-13D). Notably, no significant difference in the percentage of Th17 lineage cells between MIF098 and CTL injected, curdlan-treated SKG mice in spleen was observed, yet there was a significant increase in the percentage of Tregs (FIGS. 13E).

The therapeutic effect of MIF098 in curdlan-treated SKG mice upon reaching moderate-to-severe clinical symptoms was also tested, which would support the application of pharmacologic MIF blockade in established disease. Thus, MIF098 was injected from 4 weeks to 8 weeks post-curdlan treatment in female SKG mice (FIG. 4N). MIF098 reduced the severity of arthritis and psoriasis-like dermatitis compared to CTL-treated SKG mice post-curdlan treatment, but not blepharitis (FIGS. 4O-4R). Moreover, histological assessments demonstrated that NBF is reduced in MIF098-treated SKG mice compared to CTL-treated SKG mice post-curdlan treatment (FIG. 4S-4T). Overall, these data indicate that MIF098 can therapeutically attenuate select inflammatory pathologies and NBF in curdlan-induced disease in SKG mice.

### Adoptive Cell Transfer of Neutrophils Induces SpA-Like Pathologies in Mif KO SKG Mice

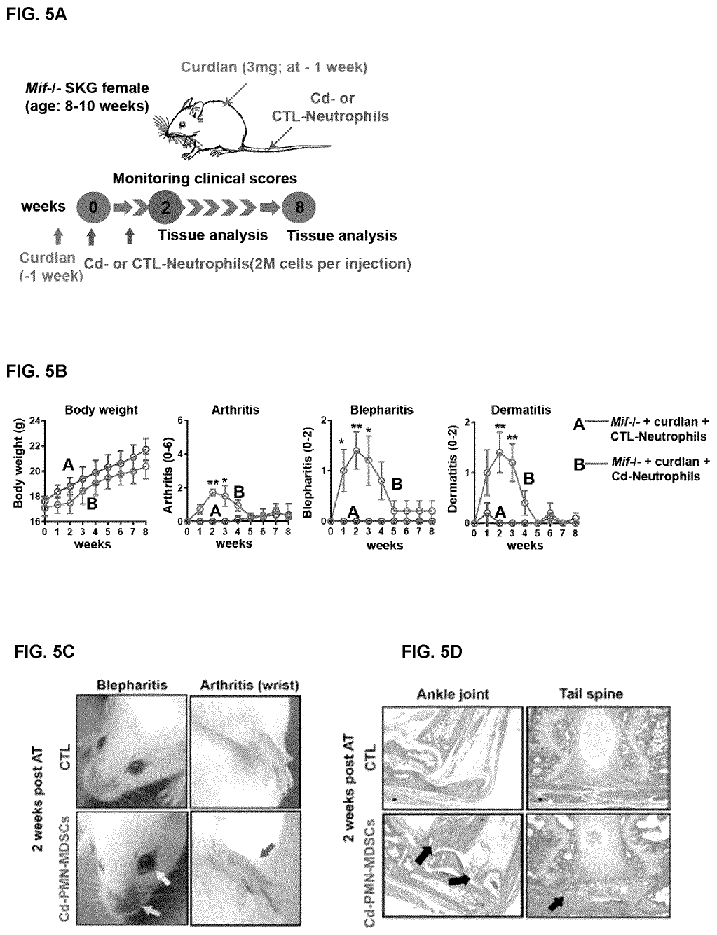

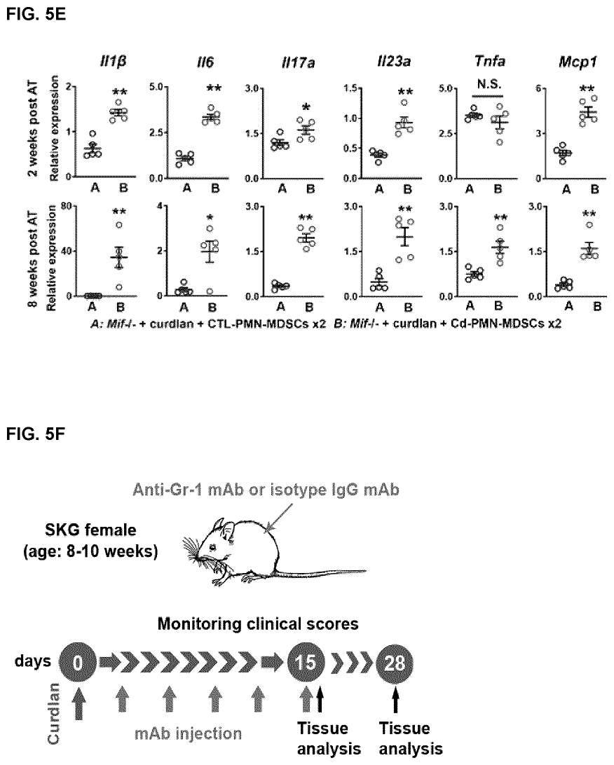

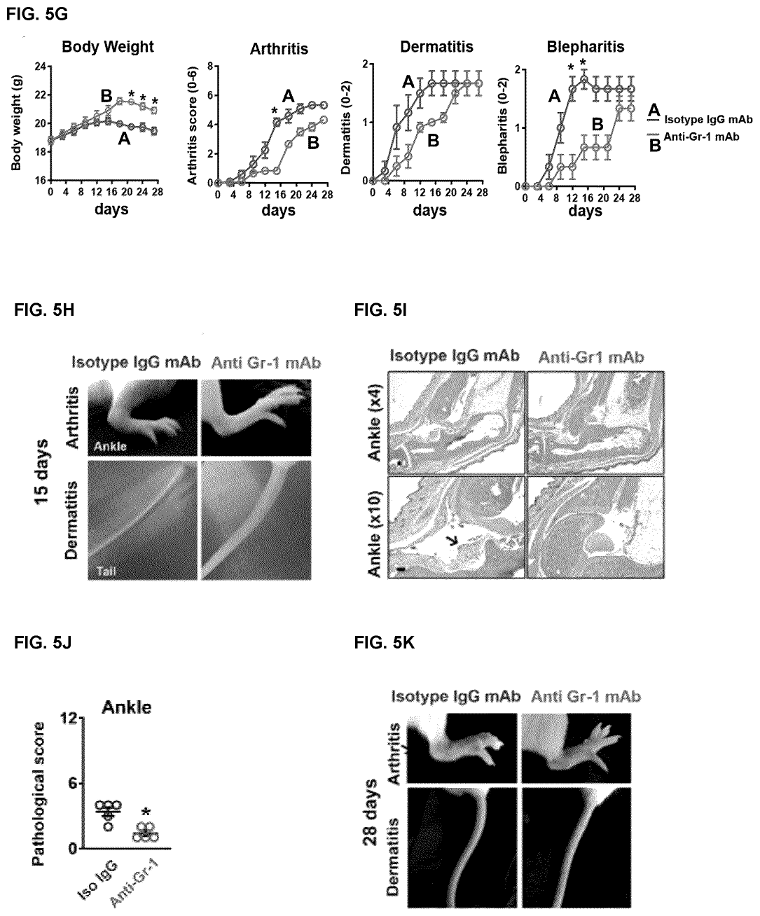

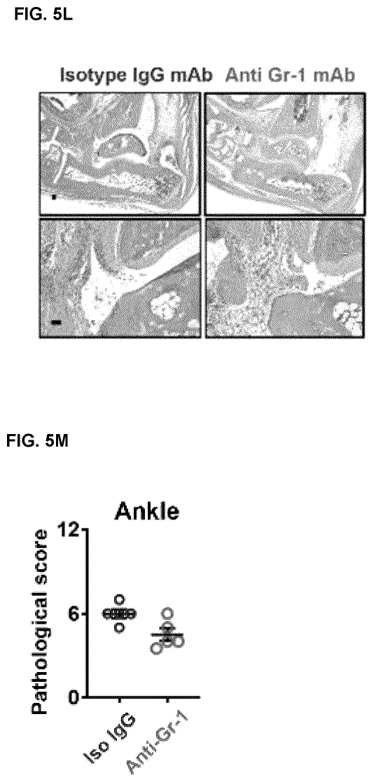

Since neutrophils were substantially expanded in curdlan-induced SKG mice and produced increased amount of MIF in vitro, adoptive transfer studies were performed using neutrophils (2×10⁶ cells/injection) obtained from curdlan-treated SKG mice, where neutrophils were transferred into curdlan-treated Mif KO SKG mice at 1 and 2 weeks post-curdlan treatment (FIG. 5A) to determine if these cells were sufficient to induce SpA-like pathologies in the absence of host MIF expression. Transfer of neutrophils from curdlan-treated SKG mice induced SpA-like clinical features including arthritis, psoriasis-like dermatitis and blepharitis until 4-5 weeks post-curdlan treatment, followed by a gradual resolving of pathologies by 8 weeks post-curdlan treatment (FIGS. 5B-5C). Evidence of infiltration of inflammatory cells into the ankles in Mif KO SKG mice injected with curdlan-neutrophils was observed compared to Mif KO SKG mice injected with control-neutrophils obtained from PBS-treated SKG mice, as assessed by histology (FIG. 5D). The expression of inflammatory markers (Il1β, Il6, Il17a, Il23a, and Mcp1) was increased in ankle joints of Mif KO SKG mice injected with curdlan-neutrophil scompared to control-neutrophils-injected mice; however, no significant difference in the expression of Tnfa was observed (FIG. 5E). Although no significant clinical differences was observed visually at 8 weeks, the expression of all inflammatory markers in ankle joints 8 weeks post-cell injection was significantly increased in Mif KO SKG mice injected with curdlan-neutrophilscompared to Mif KO SKG mice injected with control-neutrophils (FIG. 5E). These results suggest that subclinical inflammation might be ongoing in the joint. Taken together, these results suggest that curdlan-induced neutrophils are sufficient to induce SpA-like pathologies in SKG mice.

### Blocking of Neutrophils with Anti-Gr1 Monoclonal Antibody Delays the Progression of SpA-Related Symptoms

Since curdlan-induced neutrophils transferred SpA-like pathologies, whether blocking the function of neutrophils could suppress disease progression in curdlan-treated SKG mice was tested. Either anti-Gr-1 or isotype IgG monoclonal antibody (mAb) was injected into curdlan-treated SKG mice every 3 days until 15 days post-curdlan treatment (FIG. 5F). Compared to isotype-injected controls, SKG mice injected with anti-Gr-1 mAb showed reduced severity of arthritis, psoriasis-like dermatitis and blepharitis over 28 days post-curdlan treatment (FIG. 5G). Histopathological scoring for arthritis also showed decreased severity of inflammation in the ankle joints and tail spine of anti-Gr-1 mAb compared to isotype IgG mAb (FIGS. 5H-5J) at 15 days post-curdlan treatment, yet there was no clear clinical or histologic difference found at 28 days post-curdlan treatment (FIGS. 5K-5M), likely due to suspension of anti-Gr-1 treatment at 15 days post-curdlan.

### Enhancement of Treg Acquisition of a Th17 Cell-Like Phenotype under Co-Stimulation with MIF

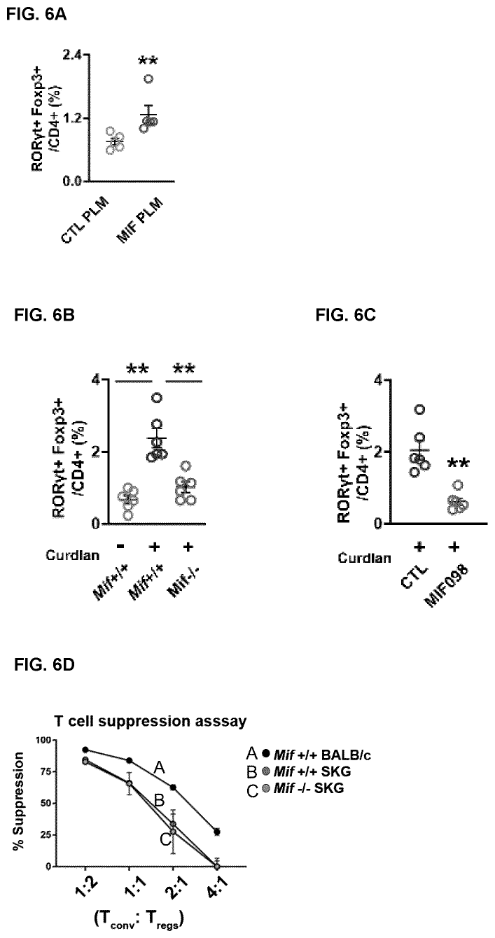

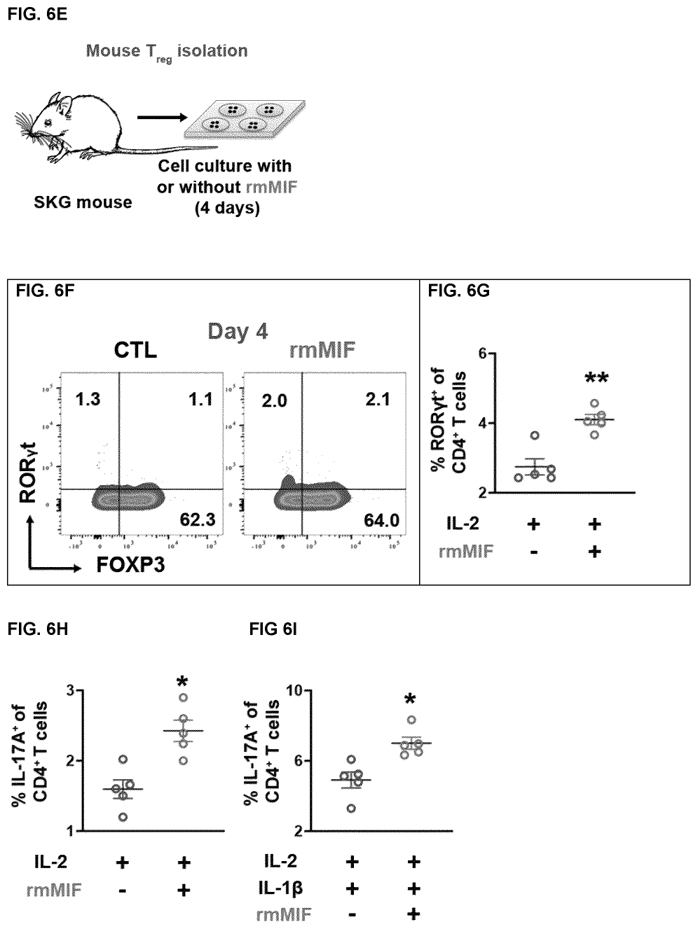

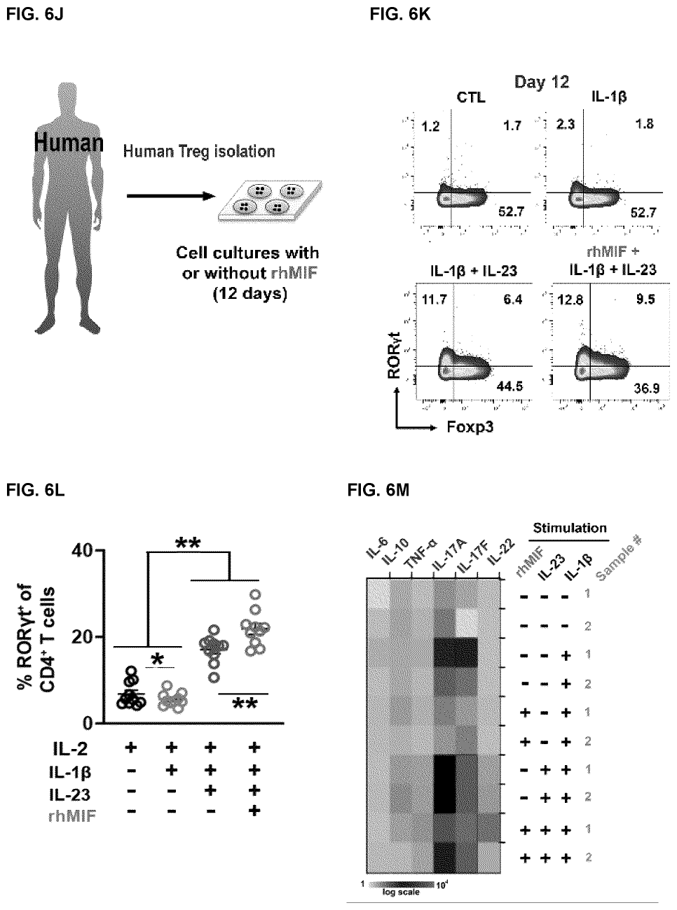

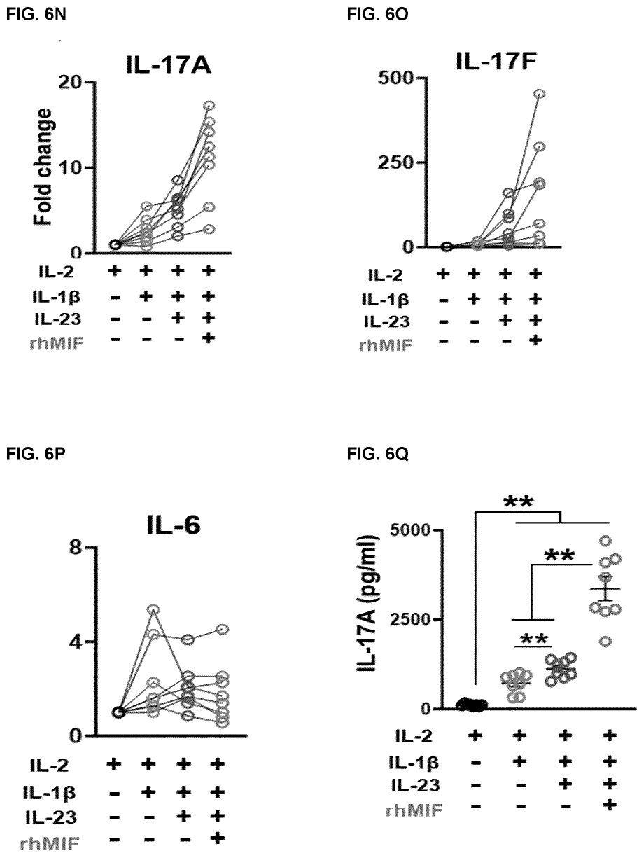

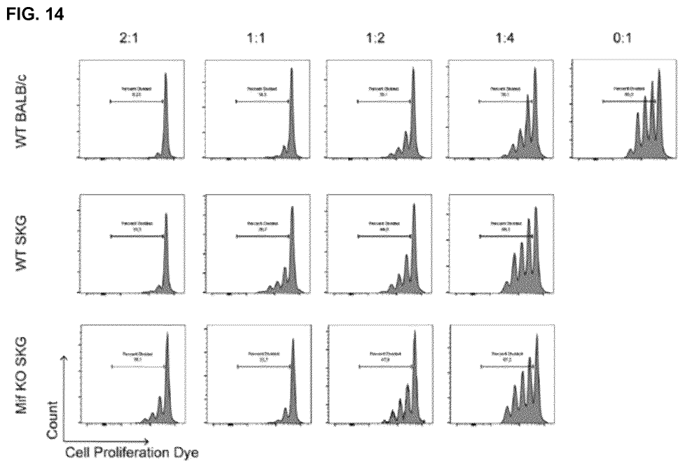

Since an imbalance in the ratio of Th17/Treg has been reported in SpA (32). Using flow cytometry, it was found that RORγt+Foxp3+CD4+ T cells were significantly expanded in curdlan-treated and MIF-overexpressing SKG mice, whereas this population was decreased with MIF098 or in Mif KO SKG mice (FIGS. 6A-6C). As these expanded cells are double positive for RORγt and Foxp3, it was hypothesized that MIF may facilitate the acquisition of a Th17 cell-like phenotype not only by differentiation from naïve CD4+ T cells, but also from Tregs. It was first confirmed that Treg suppressive function was not modified in Mif KO SKG compared to WT SKG mice (FIGS. 6D and 14). Tregs from healthy SKG mice and cultured the cells with or without rmMIF were isolated (FIG. 6E). It was observed that Tregs showed significantly increased expression of RORγt+CD4+ T cells when co-stimulated with rmMIF compared to controls without rmMIF, with enhanced expression of IL-17A (FIGS. 6F-6I).

Similar to mouse Tregs, human Tregs were isolated from healthy individuals and treated with or without rhMIF in the presence of IL1β and IL23 (FIG. 6J). Strikingly, it was observed that human Tregs co-stimulated with rhMIF increased the frequency of RORγt+CD4+ T cells compared to those from Tregs cultured without rhMIF (FIGS. 6K-6L). The secretion of IL-17A from rhMIF-treated human Tregs into the culture media was confirmed to be significantly higher than control-treated Tregs, without increased release of IL-6, a well-established Th17-inducing factor(15) (FIGS. 6M-6Q) These results suggest that MIF is a pivotal cytokine boosting type 3 immunity in both mouse and human, by enhancing Treg acquisition of a Th17 cell-like phenotype, including the upregulation of RORγt and IL-17A.

### Example 3

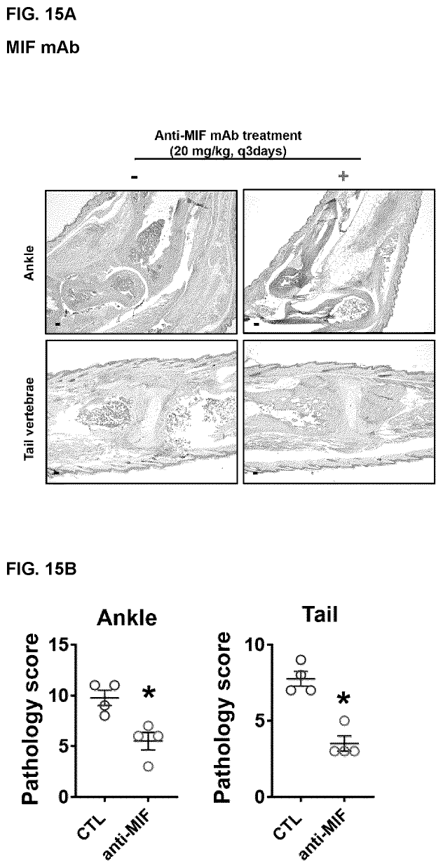

Anti-MIF antibody (IgG1, NIH IIID.9), a monoclonal antibody against MIF (anti-MIF mAb) (Leng et al., J Immunol 186, 527-38 (2011)) was administered to the mouse model described in Examples 1 and 2 and showed inhibition of arthritis (FIG. 15, A to B).

### Tables

TABLE 1
Criteria for clinical scores of blepharitis, dermatitis, and arthritis.

| Symptom | Scores |
|---|---|
| Blepharitis | Swelling, redness or discharge around eye |
| | 0 - no eyes, |
| | 1 - single eye |
| | 2 - both eyes |
| Dermatitis | Dermatitis: scaling, redness, swelling of skin |
| | 0 - no dermatitis |
| | 1- tail |
| | 1 - ear |
| Scoring system for arthritis | 0.5: Digit affected (of 4 feet) |
| (Swelling or Redness) | 1.0: Each wrist or ankle affected (of 4 joints) |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

1. V. Ranganathan, E. Gracey, M. A. Brown, R. D. Inman, N. Haroon, Pathogenesis of ankylosing spondylitis—recent advances and future directions., Nature reviews. Rheumatology 13, 359-367 (2017).
2. E. M. Gravallese, G. Schett, Effects of the IL-23-IL-17 pathway on bone in spondyloarthritis., Nature reviews. Rheumatology 14, 631-640 (2018).
3. N. Haroon, R. D. Inman, T. J. Learch, M. H. Weisman, M. Lee, M. H. Rahbar, M. M. Ward, J. D. Reveille, L. S. Gensler, The impact of tumor necrosis factor alpha inhibitors on radiographic progression in ankylosing spondylitis., Arthritis and rheumatism 65, 2645-2654 (2013).
4. L. M. Ornbjerg, C. H. Brahe, J. Askling, A. Ciurea, H. Mann, F. Onen, E. K. Kristianslund, D. Nordstrom, M. J. Santos, C. Codreanu, J. Gomez-Reino, Z. Rotar, B. Gudbjornsson, D. di Giuseppe, M. J. Nissen, K. Pavelka, M. Birlik, T. Kvien, K. K. Eklund, A. Barcelos, R. Ionescu, C. Sanchez-Piedra, M. Tomsic, A. J. Geirsson, A. G. Loft, I. van der Horst-Bruinsma, G. Jones, F. lannone, L. Hyldstrup, N. S. Krogh, M. L. Hetland, M. Ostergaard, Treatment response and drug retention rates in 24 195 biologic-naive patients with axial spondyloarthritis initiating TNFi treatment: routine care data from 12 registries in the EuroSpA collaboration., Annals of the rheumatic diseases 78, 1536-1544 (2019).
5. I. Kang, R. Bucala, The immunobiology of MIF: function, genetics and prospects for precision medicine., Nature reviews. Rheumatology 15, 427-437 (2019).
6. V. Ranganathan, F. Ciccia, F. Zeng, I. Sari, G. Guggino, J. Muralitharan, E. Gracey, N. Haroon, Macrophage Migration Inhibitory Factor Induces Inflammation and Predicts Spinal Progression in Ankylosing Spondylitis, Arthritis and Rheumatology (2017), doi:10.1002/art.40175.
7. X. Baraliakos, N. Baerlecken, T. Witte, F. Heldmann, J. Braun, High prevalence of anti-CD74 antibodies specific for the HLA class II-associated invariant chain peptide (CLIP) in patients with axial spondyloarthritis., Annals of the rheumatic diseases 73, 1079-1082 (2014).
8. N. T. Baerlecken, S. Nothdorft, G. H. Stummvoll, J. Sieper, M. Rudwaleit, S. Reuter, T. Matthias, R. E. Schmidt, T. Witte, Autoantibodies against CD74 in spondyloarthritis., Annals of the rheumatic diseases 73, 1211-1214 (2014).
9. E. Riechers, N. Baerlecken, X. Baraliakos, K. Achilles-Mehr Bakhsh, P. Aries, B. Bannert, K. Becker, J. Brandt-Jurgens, J. Braun, B. Ehrenstein, H.-H. Euler, M. Fleck, R. Hein, K. Karberg, L. Kohler, T. Matthias, R. Max, A. Melzer, D. Meyer-Olson, J. Rech, K. Rockwitz, M. Rudwaleit, R. E. Schmidt, E. Schweikhard, J. Sieper, C. Stille, U. von Hinuber, P. Wagener, H.-F. Weidemann, S. Zinke, T. Witte, Sensitivity and Specificity of Autoantibodies Against CD74 in Nonradiographic Axial Spondyloarthritis., Arthritis & rheumatology (Hoboken, N.J.) 71, 729-735 (2019).
10. G. Sogkas, K. Klose, N. Baerlecken, E. Schweikhard, T. Matthias, K. Kniesch, R. E. Schmidt, T. Witte, CD74 is a T cell antigen in spondyloarthritis., Clinical and experimental rheumatology 38, 195-202 (2020).
11. E. Gracey, D. Hromadova, M. Lim, Z. Qaiyum, M. Zeng, Y. Yao, A. Srinath, Y. Baglaenko, N. Yeremenko, W. Westlin, C. Masse, M. Muller, B. Strobl, W. Miao, R. D. Inman, TYK2 inhibition reduces type 3 immunity and modifies disease progression in murine spondyloarthritis., The Journal of clinical investigation (2020), doi:10.1172/JCI126567.
12. F. Ciccia, G. Guggino, A. Rizzo, L. Saieva, S. Peralta, A. Giardina, A. Cannizzaro, G. Sireci, G. de Leo, R. Alessandro, G. Triolo, Type 3 innate lymphoid cells producing IL-17 and IL-22 are expanded in the gut, in the peripheral blood, synovial fluid and bone marrow of patients with ankylosing spondylitis., Annals of the rheumatic diseases 74, 1739-1747 (2015).
13. J. Zhu, H. Yamane, W. E. Paul, Differentiation of effector CD4 T cell populations (*)., Annual review of immunology 28, 445-489 (2010).
14. J. Zhu, W. E. Paul, Peripheral CD4+ T-cell differentiation regulated by networks of cytokines and transcription factors., Immunological reviews 238, 247-262 (2010).
15. N. Komatsu, K. Okamoto, S. Sawa, T. Nakashima, M. Oh-hora, T. Kodama, S. Tanaka, J. A. Bluestone, H. Takayanagi, Pathogenic conversion of Foxp3+ T cells into TH17 cells in autoimmune arthritis., Nature medicine 20, 62-68 (2014).
16. N. Sakaguchi, T. Takahashi, H. Hata, T. Nomura, T. Tagami, S. Yamazaki, T. Sakihama, T. Matsutani, I. Negishi, S. Nakatsuru, S. Sakaguchi, Altered thymic T-cell selection due to a mutation of the ZAP-70 gene causes autoimmune arthritis in mice., Nature 426, 454-460 (2003).
17. M. Ruutu, G. Thomas, R. Steck, M. A. Degli-Esposti, M. S. Zinkernagel, K. Alexander, J. Velasco, G. Strutton, A. Tran, H. Benham, L. Rehaume, R. J. Wilson, K. Kikly, J. Davies, A. R. Pettit, M. A. Brown, M. A. McGuckin, R. Thomas, beta-glucan triggers spondylarthritis and Crohn's disease-like ileitis in SKG mice., Arthritis and rheumatism 64, 2211-2222 (2012).
18. M. A. Rahman, R. Thomas, The SKG model of spondyloarthritis., Best practice & research. Clinical rheumatology 31, 895-909 (2017).
19. H. Jeong, E.-K. Bae, H. Kim, D. H. Lim, T.-Y. Chung, J. Lee, C. H. Jeon, E.-M. Koh, H.-S. Cha, Spondyloarthritis features in zymosan-induced SKG mice., Joint, bone, spine: revue du rhumatisme 583-591 (2018).
20. D. Baeten, M. Østergaard, J. C.-C. Wei, J. Sieper, P. Järvinen, L.-S. Tam, C. Salvarani, T.-H. Kim, A. Solinger, Y. Datsenko, C. Pamulapati, S. Visvanathan, D. B. Hall, S. Aslanyan, P. Scholl, S. J. Padula, Risankizumab, an IL-23 inhibitor, for ankylosing spondylitis: results of a randomised, double-blind, placebo-controlled, proof-of-concept, dose-finding phase 2 study., Annals of the rheumatic diseases 77, 1295-1302 (2018).
21. W. Hueber, B. E. Sands, S. Lewitzky, M. Vandemeulebroecke, W. Reinisch, P. D. R. Higgins, J. Wehkamp, B. G. Feagan, M. D. Yao, M. Karczewski, J. Karczewski, N. Pezous, S. Bek, G. Bruin, B. Mellgard, C. Berger, M. Londei, A. P. Bertolino, G. Tougas, S. P. L. Travis, Secukinumab, a human anti-IL-17A monoclonal antibody, for moderate to severe Crohn's disease: unexpected results of a randomised, double-blind placebo-controlled trial., Gut 61, 1693-1700 (2012).
22. Y. Mizue, S. Ghani, L. Leng, C. McDonald, P. Kong, J. Baugh, S. J. Lane, J. Craft, J. Nishihira, S. C. Donnelly, Z. Zhu, R. Bucala, Role for macrophage migration inhibitory factor in asthma., Proceedings of the National Academy of Sciences of the United States of America 102, 14410-14415 (2005).
23. S.-A. Yoo, L. Leng, B.-J. Kim, X. Du, P. v Tilstam, K. H. Kim, J.-S. Kong, H.-J. Yoon, A. Liu, T. Wang, Y. Song, M. Sauler, J. Bernhagen, C. T. Ritchlin, P. Lee, C.-S. Cho, W.-U. Kim, R. Bucala, MIF allele-dependent regulation of the MIF coreceptor CD44 and role in rheumatoid arthritis., Proceedings of the National Academy of Sciences of the United States of America 113, E7917—E7926 (2016).
24. A. Nakamura, Y. R. Rampersaud, S. Nakamura, A. Sharma, F. Zeng, E. Rossomacha, S. A. Ali, R. Krawetz, N. Haroon, A. v. Perruccio, N. N. Mahomed, R. Gandhi, J. S. Rockel, M. Kapoor, MicroRNA-181a-5p antisense oligonucleotides attenuate osteoarthritis in facet and knee joints Annals of the Rheumatic Diseases (2018), doi:10.1136/annrheumdis-2018-213629.
25. A. Nakamura, Y. R. Rampersaud, A. Sharma, S. J. Lewis, B. Wu, P. Datta, K. Sundararajan, H. Endisha, E. Rossomacha, J. S. Rockel, I. Jurisica, M. Kapoor, Identification of microRNA-181a-5p and microRNA-4454 as mediators of facet cartilage degeneration, JCI Insight (2016), doi:10.1172/jci.insight.86820.
26. P. R. Taylor, S. V. Tsoni, J. A. Willment, K. M. Dennehy, M. Rosas, H. Findon, K. Haynes, C. Steele, M. Botto, S. Gordon, G. D. Brown, Dectin-1 is required for beta-glucan recognition and control of fungal infection., Nature immunology 8, 31-38 (2007).
27. D. M. Underhill, E. Rossnagle, C. A. Lowell, R. M. Simmons, Dectin-1 activates Syk tyrosine kinase in a dynamic subset of macrophages for reactive oxygen production., Blood 106, 2543-2550 (2005).
28. R. Das, M.-S. Koo, B. H. Kim, S. T. Jacob, S. Subbian, J. Yao, L. Leng, R. Levy, C. Murchison, W. J. Burman, C. C. Moore, W. M. Scheld, J. R. David, G. Kaplan, J. D. MacMicking, R. Bucala, Macrophage migration inhibitory factor (MIF) is a critical mediator of the innate immune response to Mycobacterium tuberculosis., Proceedings of the National Academy of Sciences of the United States of America 110, E2997-3006 (2013).
29. S. M. Bal, K. Golebski, H. Spits, Plasticity of innate lymphoid cell subsets., Nature reviews. Immunology (2020), doi:10.1038/s41577-020-0282-9.
30. K. Hirota, M. Hashimoto, Y. Ito, M. Matsuura, H. Ito, M. Tanaka, H. Watanabe, G. Kondoh, A. Tanaka, K. Yasuda, M. Kopf, A. J. Potocnik, B. Stockinger, N. Sakaguchi, S. Sakaguchi, Autoimmune Th17 Cells Induced Synovial Stromal and Innate Lymphoid Cell Secretion of the Cytokine GM-CSF to Initiate and Augment Autoimmune Arthritis., Immunity 48, 1220-1232.e5 (2018).
31. A. v Misharin, C. M. Cuda, R. Saber, J. D. Turner, A. K. Gierut, G. K. 3rd Haines, S. Berdnikovs, A. Filer, A. R. Clark, C. D. Buckley, G. M. Mutlu, G. R. S. Budinger, H. Perlman, Nonclassical Ly6C(−) monocytes drive the development of inflammatory arthritis in mice., Cell reports 9, 591-604 (2014).
32. I. B. McInnes, A. Kavanaugh, A. B. Gottlieb, L. Puig, P. Rahman, C. Ritchlin, C. Brodmerkel, S. Li, Y. Wang, A. M. Mendelsohn, M. K. Doyle, Efficacy and safety of ustekinumab in patients with active psoriatic arthritis: 1 year results of the phase 3, multicentre, double-blind, placebo-controlled PSUMMIT 1 trial., Lancet (London, England) 382, 780-789 (2013).

1. A method of treating spondyloarthritis (SpA) comprising administering a MIF inhibitor to a subject in need thereof.

2. The method of claim 1, wherein the SpA is early SpA or axial SpA.

3. The method of claim 1, wherein the treating is for inhibiting new bone formation or other radiologically detectable manifestations of SpA.

4. The method of claim 1, wherein the SpA is late SpA.

5. The method of claim 1, wherein the subject is treated during a flare or remission.

6. (canceled)

7. The method of claim 1, wherein the SpA is ankylosing spondylitis, non-radiographic axial SpA, reactive arthritis (RA), IBD-related SpA, psoriatic arthritis (PsA), juvenile-onset idiopathic arthritis (JIA), undifferentiated SpA (USpA).

8. The method of any one of claim 1, wherein the method is for treating an extra-articular symptoms.

9. The method of claim 8, wherein the extra-articular symptoms is an eye manifestation, optionally uveitis or iritis, a skin manifestation, optionally psoriasis, a gut manifestation optionally IBD, optionally Crohn's disease or ulcerative colitis.

10-11. (canceled)

12. A method of inhibiting new bone formation in a subject with SpA comprising administering a MIF inhibitor to the subject.

13. The method of claim 1, wherein the MIF inhibitor is or comprises a compound of Formula I, II, IIA or B in WO2010021693 and/or U.S. Pat. No. 9,643,922 (MIF Modulators).

14. The method of claim 1, wherein the MIF inhibitor is or comprises MIF098 or a MIF098 analog, salt or derivative thereof or Ibudilast or a Ibudilast analog, salt or derivative thereof or is an anti-MIF antibody or binding fragment thereof.

15. (canceled)

16. The method of claim 14, wherein the anti-MIF antibody or binding fragment thereof is a humanized monoclonal antibody, human antibody or a binding fragment thereof.

17. The method of claim 16, wherein the anti-MIF antibody or binding fragment is imalumab or an anti-CD74 antibody or binding fragment thereof.

18-19. (canceled)

20. The method of claim 17, wherein the anti-CD74 antibody or binding fragment thereof is milatuzumab or a binding fragment thereof.

21. (canceled)

22. The method of claim 1, wherein the MIF inhibitor is in the form of a solid dosage form or a liquid dosage form.

23. (canceled)

24. The method of claim 22, wherein the dosage form is formulated for oral administration, for intravenous administration or for subcutaneous administration.

25-26. (canceled)

27. The method of claim 1, wherein the MIF inhibitor is administered contemporaneously with another SpA therapy.

28. The method of claim 27, wherein the SpA therapy is a TNF inhibitor, optionally an anti-TNFa antibody, or an IL-17 inhibitor, optionally an anti-II-17 antibody.

29-111. (canceled)

112. The method of claim 1, wherein the MIF inhibitor is or comprises a compound of Formula B: 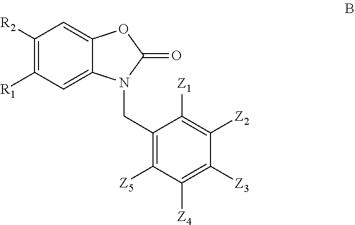
or a pharmaceutically acceptable salt thereof,
wherein R1 is hydroxyl, optionally substituted C1-C8 alkyl, optionally substituted C1-C10 alkoxy, F, Cl, or (CH2)j—OH; and R2 is H; or R1 is H, and R2 is hydroxyl, optionally substituted C1-C8 alkyl, optionally substituted C1-C10 alkoxy, F, Cl, or (CH2)j—OH; Z1 is hydroxyl, optionally substituted C1-C8 alkyl, C1 alkoxy, F, Cl, or (CH2)j—OH; Z2 is H; and Z3 is H; or Z1 is H; Z2 is hydroxyl, optionally substituted C1-C8 alkyl, optionally substituted C1-C10 alkoxy, F, Cl, or (CH2)j—OH; and Z3 is H; or Z1 is H; Z2 is H; and Z3 is hydroxyl, optionally substituted C1-C8 alkyl, optionally substituted C1-C10 alkoxy, F, Cl, or (CH2)j—OH; Z4 is H; Z5 is H; and each j is independently 0, 1, 2, 3, 4 or 5.
